(19) United States
(12) Patent Application Publication
(10) Pub. No.: US 2015/0038682 A1
(43) Pub. Date: Feb. 5, 2015

(54) ANTIBODIES OR FUSION PROTEINS MULTIMERIZED VIA HOMOMULTIMERIZING PEPTIDE

(71) Applicant: JN BIOSCIENCES LLC, Mountain View, CA (US)

(72) Inventors: Naoya Tsurushita, Palo Alto, CA (US); J. Yun Tso, Menlo Park, CA (US)

(73) Assignee: JN BIOSCIENCES LLC, MOUNTAIN VIEW, CA (US)

(21) Appl. No.: 14/449,076

(22) Filed: Jul. 31, 2014

Related U.S. Application Data
(60) Provisional application No. 61/861,928, filed on Aug. 2, 2013.

(51) Int. Cl. C07K 16/28

(52) U.S. Cl. CPC C07K 16/2878; C07K 16/2803; C07K 2317/73; C07K 2317/24; C07K 2317/21
USPC 530/387.3

(57) ABSTRACT

The invention provides antibodies or fusion proteins with modified heavy chain IgG constant regions that promote assembly of multimeric complexes. Within an antibody or fusion protein unit there are two heavy chains each including at least CH2 and CH3 regions. The two heavy chains bear complementary modifications (e.g., knob and hole) to promote coupling of the heavy chains within a unit. One and only one of the heavy chains in a unit is fused at its C-terminus to a homomultimerizing peptide. The presence of the homomultimerizing peptide promotes association between units. For example, if the homomultimerizing peptide is a homotrimerizing peptide it promotes association of three units to form a trimeric complex.

### CROSS-REFERENCE TO RELATED APPLICATION

The present application is a nonprovisional and claims the benefit of 61/861,928 filed Aug. 2, 2013, incorporated by reference in its entirety for all purposes.

### REFERENCE TO A SEQUENCE LISTING

The present application includes sequences provided in a txt filed designated 449846SEQLST, of 99 kb, created Jul. 31, 2014, which is incorporated by reference.

### BACKGROUND

Antibodies are glycoproteins produced by B cells that play an essential role in the immune system (Schroeder et al., J. Allergy Clin. Immunol. 125:S41-S52, 2010). Five classes of antibodies, namely IgM, IgD, IgG, IgA and IgE, are produced in mammals. In humans, four subclasses of IgG (IgG1, IgG2, IgG3 and IgG4) and two subclasses of IgA (IgA1 and IgA2) antibodies are produced. Each antibody is composed of two identical light chains and two identical heavy chains in the monomeric form. These four chains are connected to one another by a combination of covalent and non-covalent bonds, and form a Y-shaped molecule. There are two types of light chains, kappa and lambda, in mammals. Several different types of heavy chains exist that define the class of an antibody. In humans, the μ heavy chain is incorporated in IgM, the delta heavy chain in IgD, the gamma-1 heavy chain in IgG1, the gamma-2 heavy chain in IgG2, the gamma-3 heavy chain in IgG3, the gamma-4 heavy chain in IgG4, the alpha-1 heavy chain in IgA1, the alpha-2 heavy chain in IgA2, and the epsilon heavy chain in IgE. A monomeric form of these antibodies has two antigen binding sites, and thus is bivalent for antigen binding. Although IgG, IgD and IgE are exclusively produced as a monomer, IgM is produced as a hexamer, and thus is dodecavalent for antigen binding, in the absence of J chains, and forms a decavalent pentamer when J chains are present (Gilmour et al., Trans. Med. 18:167-174, 2008). IgA forms a tetravalent dimer with a J chain, whereas IgA is a monomer when J chains are absent, although spontaneous formation of dimeric IgA without J chains has been reported (Johansen et al., Scand. J. Immunol. 52:240-248, 2000).

The U.S. Food and Drug Administration had approved thirty-three monoclonal antibodies as human therapeutics by the end of 2012. All of these therapeutic antibodies are IgG antibodies or derivatives thereof. Besides specific antigen binding, IgG antibodies elicit various biological functions mediated by the Fc region (Schroeder et al. supra; Desjarlais et al., Exp. Cell Res. 317:1278-1285, 2011). In humans, cell-bound IgG1 and IgG3 antibodies mediate antibody-dependent cell-mediated cytotoxicity (ADCC) by binding of the Fc region to Fcγ receptor type III (CD16) expressed on NK cells (Hulett et al., Adv. Immunol. 57:1-127, 1994). Likewise, cell-bound IgG1 and IgG3 antibodies can efficiently trigger complement-dependent cytotoxicity (CDC) by the interaction of the Fc region with complement components (Bindon et al., J. Exp. Med. 168:127-142, 1988).

The Fc region of all four subclasses of human IgG antibodies binds to the neonatal Fc receptor (FcRn), which is a heterodimer composed of a transmembrane α chain and β2-microglubulin, in a pH-dependent manner, resulting in rescuing IgG antibodies internalized by pinocytosis from catabolic degradation in lysosomes and allowing their recycling to the circulation (Ghetie et al., Annu. Rev. Immunol. 18:739-766, 2000). IgG antibodies therefore exhibit slow clearance from the circulation which results in a long serum half-life, typically 23 days, in humans (Kindt et al., Chapter 4, Kuby Immunology, Sixth Edition, W. H. Freeman & Co., 2006). In addition, the Fc region of IgG antibodies bind to Protein A (except for IgG3) and Protein G, so that purification of IgG antibodies by Protein A or Protein G affinity chromatography is possible (Andrew et al., Unit 2.7, Chapter III, Current Protocols in Immunology, John Wiley & Sons, Inc. 1997).

Dimerization of specific molecules on the cell surface can often trigger one or more biological responses. Binding of monoclonal IgG antibodies to PSMA (prostate-specific membrane antigen) proteins on the cell surface increases the rate of PSMA internalization (Liu et al., Cancer Res. 58:4055-4060, 1998). Internalization and down-regulation of a type I transmembrane protein MUC1 is triggered by binding to a mouse IgG1 antibody (Hisatsune et al., Biochem. Biophys. Res. Commun. 388:677-382, 2009). Monoclonal antibodies against c-Met dimerize c-Met proteins on the cell surface and initiate intracellular signals resulting in cell proliferation (Prat et al., J. Cell Sci. 111:237-247, 1998). Likewise, a monoclonal anti-EPO receptor antibody can function as an agonist for cell growth by homodimerization of EPO receptors on the surface (Schneider et al., Blood 89:473-482, 1997). Antibody-mediated dimerization of Death Receptor 5 (DR5), a member of tumor necrosis factor receptor (TNFR) super-family, on the cell surface, however, does not always trigger signal transduction, while multimerization of DR5 proteins by a mixture of mouse monoclonal anti-DR5 IgG antibody and goat anti-mouse IgG polyclonal antibody, for example, induces signal transduction in the cytoplasm and triggers apoptosis (Griffith et al., J. Immunol. 162:2597-2605, 1999).

IgM antibodies exist as pentamers with J chains and hexamers without J chains (Gilmour et al., supra). In contrast to IgG antibodies, which are only capable of dimerizing antigens, IgM can multimerize cell surface proteins due to its decavalent or dodecavalent antigen binding capability. Monoclonal IgM antibodies with specificity for Fas, a member of the TNFR superfamily (Cosman, Stem Cells 12:440-455, 1994), can efficiently induce apoptosis of Fas-expressing cells due to multimerization of Fas proteins on the surface (Yonehara et al., J. Exp. Med. 169:1747-1756, 1989) while anti-Fas IgG antibodies do not unless they are cross-linked (Matsuno et al., J. Rheumatol. 29:1609-1614, 2002). Compared to IgG, IgM exhibits a much shorter circulation half-life, typically 5 days in humans, because of its inability to bind to FcRn (Kindt et al., supra). IgM antibodies are also unable to mediate ADCC due to the lack of binding to CD16. In addition, the lack of binding to Protein A and Protein G by IgM makes it impossible to purify IgM by Protein A and Protein G affinity chromatography, respectively (Gautam et al., Biotechnol. Adv. 29:84-849, 2011).

A variety of structural formats have been utilized in an attempt to generate novel forms of multivalent antibodies. Recent advances in the engineering of multivalent antibodies are summarized in a review paper of Cuesta et al. (Trends Biotech., 28:355-362, 2010). Preferred multivalent IgG antibodies are able to multimerize antigens efficiently on the cell surface. It is also important that the properties mediated by the Fc region of gamma heavy chains, such as ADCC, CDC, opsonization, pH-dependent FcRn binding, and the ability to bind to Protein A and Protein G, are maintained in such multivalent IgG antibodies.

To generate a multivalent IgG antibody, Caron et al. (J. Exp. Med., 176:1191-1195, 1992) introduced a serine-to-cysteine substitution at the fourth position from the carboxyl terminal of human gamma-1 heavy chain in the humanized anti-CD33 IgG1/kappa antibody, HuG1-M195. Such modified HuG1-M195, termed Hd-IgG, was purified and subjected to Ellman's Reagent (Pierce Chemical Co., Rockford, Ill.) for crosslinking and then blocking of excess sulfhydryl sites. Monomeric HuG1-M195 was eliminated from Hd-IgG by phenyl Sepharose column chromatography. The resultant Hd-IgG showed a dramatic improvement in the ability to internalize CD33 molecules and was more potent than HuG1-M195 at ADCC and CDC. Miller et al. (J. Immunol., 170:4854-4861, 2003) constructed a tetravalent IgG antibody by duplicating the VH-CH1 region in the heavy chain of the humanized anti-HER2 IgG1 monoclonal antibody, hu4D5. The modified gamma heavy chain was composed of, from the N-terminus to the C-terminus, the VH, CH1, VH, CH1, hinge, CH2 and CH3 regions. One light chain bound to each of the four VH-CH1 regions in the modified IgG, forming a tetravalent hu4D5 antibody (TA-HER2). TA-HER2 was internalized more rapidly than the parental bivalent hu4D5 on HER2-expressing cells. Miller et al. (supra) also constructed a tetravalent anti-DR5 IgG antibody, termed TA-DR5, in the same heavy chain format as in TA-HER2. TA-DR5 triggered apoptosis at ^{˜}100-fold lower concentration than the parental bivalent anti-DR5 IgG monoclonal antibody.

Rossi et al. (Cancer Res., 68:8384-8392, 2008) reported the construction of a hexavalent anti-CD20 IgG antibody, designated Hex-hA20, using the Dock-and-Lock method. To generate Hex-hA20, which was composed of six Fab and two Fc regions, two components were constructed and separately produced in mammalian cells. First, the anchoring domain of the A-kinase anchoring proteins (AD) was genetically fused to the carboxyl terminus of the heavy chain in the humanized anti-CD20 IgG1 antibody, hA20. This construct was designated CH3-AD2-IgG-hA20. Second, the docking domain of the cyclic AMP-dependent protein kinase (DDD) was genetically fused to the carboxyl terminus of the Fab fragment of h20. This construct was designated CH1-DDD2-Fab-hA20. CH3-AD2-IgG-hA20 and CH1-DDD2-Fab-hA20 were purified by Protein A and Protein L affinity chromatography, respectively. Hex-hA20 was obtained by mixing purified CH3-AD2-IgG-hA20 and CH1-DDD2-Fab-hA20 under redox conditions followed by purification with Protein A. Hex-h20 inhibited proliferation of CD20-expressing B lymphoma cells lines without the need for a cross-linking antibody. Hex-h20 retained the ADCC activity of hA20, but lost the CDC activity.

Yoo et al. (J. Biol. Chem., 47:33771-33777, 1999) constructed variant human anti-DNS IgG2 antibodies in which part of the gamma-2 heavy chain was replaced with the corresponding part of the human alpha-1 heavy chain. In the construct termed γγγ-αtp, the 18-amino acid polypeptide present in the C-terminus of the human alpha-1 heavy chain, termed αtp (also called alpha tailpiece), was attached at the C-terminus of the human gamma-2 heavy chain. The γγγ-αtp construct was further modified to generate the following three variant IgG2 antibodies. In αγγ-αtp, the CH1 region of the gamma-2 heavy chain was replaced with the counterpart of the human alpha-1 heavy chain. In ααγ-αtp, the CH1, hinge and CH2 regions were replaced with the counterparts of the human alpha-1 heavy chain. In γαγ-αtp, the hinge and CH2 regions were replaced with the counterparts of the human alpha-1 heavy chain. These constructs were stably expressed in the mouse myeloma cell line Sp2/0 producing J chains. Each of purified γγγ-αtp, αγγ-αtp, ααγ-αtp and γαγ-αtp antibodies was a mixture of monomers, dimers, trimers, tetramers, pentamers and hexamers. The combined percentage of hexamers and pentamers in the mixture was 20% for γγγ-αtp, 25% for αγγ-αtp, 45% for ααγ-αtp, and 32% for γαγ-αtp.

Sorensen et al. (J. Immunol. 156:2858-2865, 1996) generated multivalent antibodies based on a human monoclonal anti-NIP (3-nitro-4-hydroxy-5-iodophenulacetic acid) IgG3 antibody variant in which the first, second and third hinge region are deleted. The gamma-3 heavy chain gene of this variant IgG3 antibody was modified in two locations. First, the 18-amino acid polypeptide present in the C-terminus of the human μ heavy chain, termed μtp (also called mu tailpiece), was attached at the C-terminus of the heavy chain. Second, a leucine residue at position 309 in the CH2 region was changed to a cysteine residue. Such modified monoclonal IgG3 antibody, called IgGL309Cμtp, was expressed in the mouse myeloma cell line J558L producing J chains, and purified using an NIP-Sepharose column. The secretion level was reported to be poorer for IgGL309Cμtp than for the parental IgG3 antibody, and a large fraction of IgGL309Cμtp was retained intracellularly. The size analysis showed that pentamers and hexamers constituted 81% of purified IgGL309Cμtp.

Sorensen et al. (Int. Immunol., 12:19-27, 2000) also modified the same human anti-NIP IgG3 antibody variant as described above by substituting the CH2 and CH3 regions of the gamma-3 heavy chain with the CH3 and CH4 regions, including μtp, of the human μ heavy chain. The heavy chain of such modified IgG3/IgM hybrid molecules, termed IgG-Cμ3-Cμ4, is composed of, from the N-terminus, the anti-NIP VH region, the CH1 and fourth hinge region of the human gamma-3 heavy chain, and the CH3 and CH4 regions, including μtp, of the human μ heavy chain. IgG-Cμ3-Cμ4 was expressed in J558L cells producing J chains and purified using an NIP-Sepharose column. Hexamers and pentamers constituted 14.0% and 66.7%, respectively, in purified IgG-Cμ3-Cμ4. Since IgG-Cμ3-Cμ4 does not have the CH2 and CH3 regions of the human gamma-3 heavy chain, it will lack Fcγ-mediated properties such as ADCC, pH-dependent FcRn binding, and the ability to bind to Protein A and Protein G.

There is a need of multimeric IgG antibodies, which are capable of inducing apoptosis, cytostasis and/or intracellular signal transduction by efficient cross-linking of cell surface proteins, such as TNF receptor family members (Hehlgans and Pfeffer, Immunol. 115:1-20, 2005; Mahmood and Shukla, Exp. Cell Res. 316:887-899, 2010), without losing Fcγ-mediated functions, such as ADCC, CDC, opsonization, long serum half-life and binding to protein A and protein G.

### SUMMARY OF THE CLAIMED INVENTION

The invention provides an antibody or fusion protein comprising first and second heavy chain constant regions associated with one another as a heterodimer, each chain comprising IgG CH2 and CH3 regions, and one of the chains comprising a homomultimerizing peptide linked to the C-terminus of the CH3 region. The homomultimerizing peptide can be for example, a dimerizing, a trimerizing peptide, a tetramerizing peptide or a pentamerizing peptide. The antibody or fusion can be an antibody further comprising first and second heavy chain variable regions fused to the first and second heavy chain constant regions and first and second light chains associated with the first and second heavy chains.

The antibody or fusion protein can be a dimeric fusion protein further comprising first and second heterologous proteins fused to the first and second heavy chain constant regions. The heterologous proteins can be an extracellular domain of a receptor and/or a ligand to a receptor. The first and second constant regions can further comprise and IgG hinge region and the heterologous proteins are linked to the IgG hinge regions of the first and second constant regions of the constant region via one or more flexible linkers, such as Gly-Gly-Ala-Ala.

In some antibodies or fusion proteins, the first and second heavy chains incorporate modifications of natural IgG sequences promoting formation of the heterodimer. For example, the first heavy chain can incorporate a hole and the second heavy chain a knob, wherein coupling of the knob to the hole promotes formation of the heterodimer. Optionally, the first and second heavy chains each comprises human IgG1 CH2 and CH3 regions and the first heavy chain has T366S, L368A and Y407V mutations, and the second heavy chain has a T366W mutation, amino acids being numbered by the EU numbering convention. Optionally, a trimerizing peptide is linked to the CH3 domain of the second heavy chain.

In some antibodies or fusion proteins, a trimerizing peptide comprises an isoleucine zipper or extracellular domain of a TNF family member or tetranectin.

In some antibodies, the first and second heavy chain variable regions are the same and in others the first and second heavy chain variable regions are different. In some antibodies, the first and second heavy chain variable regions are from antibodies binding to different targets. In some antibodies, first and second light chains are the same and in others different, for example from antibodies binding to different targets.

The antibodies or fusion proteins described above can exist in trimeric form, in which three units of the antibody or fusion protein form a trimer via association of the trimerizing peptides of the units.

The antibodies or fusion proteins described above can exist in tetrameric form, in which four units of the antibody or fusion protein form a tetramer via association of the tetramerizing peptides of the units.

The antibodies or fusion proteins described above can exist in pentameric form in which five units of the antibody or fusion protein form a pentamer via association of the pentamerizing peptides of the units.

The invention further provides a trimeric complex including three units of an antibody or fusion protein, each unit comprising first and second heavy chain constants regions associated with one another as a heterodimer, each chain comprising IgG CH2 and CH3 regions, and one of the chains comprising a trimerizing peptide linked to the C-terminus of the CH3 region, wherein the units are associated as a the trimeric complex via trimerizing of the trimerizing peptides on the units. Optionally, each of the three units is an antibody, further comprising first and second heavy chain variable regions fused to the first and second heavy chain constant regions and first and second light chains associated with the first and second heavy chains.

The invention further provides a multimeric complex including multiple units of an antibody or fusion protein, each unit comprising first and second heavy chain constants regions associated with one another as a heterodimer, each chain comprising IgG CH2 and CH3 regions, and one of the chains comprising a multimerizing peptide linked to the C-terminus of the CH3 region, wherein the units are associated as a the multimeric complex via multimerizing of the multimerizing peptides of the units.

In some antibodies or fusion proteins or trimeric or multimeric complexes, the IgG CH2 and CH3 regions are human IgG. Some antibodies or fusion proteins, or trimeric or multimeric complexes further comprise human IgG CH1 and hinge regions. In some antibodies or fusion proteins, or trimeric or multimeric complexes the human IgG CH1, hinge, CH2 and CH3 regions are human IgG1. In some antibodies or fusion proteins, or trimeric or multimeric complexes the human IgG CH1, hinge, CH2 and CH3 regions are human IgG2. In some antibodies or fusion proteins, or trimeric or multimeric complexes the human IgG CH1, hinge, CH2 and CH3 regions are human IgG3. In some antibodies or fusion proteins, or trimeric or multimeric complexes, the human IgG CH1, hinge, CH2 and CH3 regions are human IgG4.

Some antibodies or fusion proteins, or trimeric or multimeric complexes specifically binds to a Death Receptor family protein and induces apoptosis of cells bearing the protein, such as DR4. Some antibodies or fusion proteins, or trimeric or multimeric complexes specifically bind to a TNF receptor family protein and induces apoptosis or cytostasis of cells bearing the protein. Some antibodies or fusion proteins, or trimeric or multimeric complexes specifically binds protein G, specifically binds protein A, exhibits ADCC, CDC and/or opsonization. In some antibodies or fusion proteins, or trimeric or multimeric complexes the CH1 region, if present, and the hinge region, and CH2 and CH3 regions are human IgG1 regions, and the antibody or fusion protein specifically binds protein G, and specifically binds protein A. Some antibodies or fusion proteins, or trimeric or multimeric complexes exhibits ADCC, CDC and opsonizaton. In some antibodies or fusion proteins, or trimeric or multimeric complexes the CH1 region if present, and the hinge, CH2 and CH3 regions are human IgG2 or IgG4 regions and the antibody or fusion protein specifically binds protein G and specifically binds protein A.

In some antibodies, or trimeric or multimeric complexes, the antibody is a humanized, chimeric, veneered or human antibody. Some antibodies or fusion proteins, or trimeric or multimeric complexes specifically bind the extracellular domain of a receptor, such as CD79a, CD30, DR5 or DR4. Some fusion proteins or trimeric or multimeric complexes comprise an extracellular domain of a TNF-alpha receptor, LFA-3 or an IL-1 receptor or a TRAIL protein.

Some antibodies or fusion proteins, or trimeric or multimeric complexes are conjugated to a toxic moiety, optionally cytotoxic.

Some antibodies or trimeric or multimeric complexes specifically bind to CD40, OX40, 4-1BB, GITR or CD27.

Some antibodies or trimeric or multimeric complexes specifically bind to a TNF receptor superfamily member expressed from a cell thereby inducing trimerization of the receptor and intracellular signal transduction via the receptor. Exemplary TNF receptor superfamily member include TNFRI (CD120a), TNFRII (CD120b), LtβR (lymphotoxin beta receptor), OX40 (CD134), CD40, FAS (CD95), CD27, CD30, 4-1BB (CD137), DR3, DR4 (CD261), DR5 (CD262), DR6 (CD358), DcR1 (CD263), DcR2 (CD264), DcR3, RANK (CD265), OPG, Fn14 (CD266), TACI (CD267), BAFFR (CD268), BCMA (CD269), HVEM (CD270), LNGFR (CD271), GITR (CD357), TROY, RELT, EDAR or XEDAR.

The invention further provides a pharmaceutical composition comprising an antibody or fusion protein or trimeric or multimeric complex as defined above.

The invention further provides a method of treating cancer comprising administering to a patient having or at risk of cancer an effective regime of an antibody or fusion protein or trimeric or multimeric complex thereof as defined above.

The invention further provides a method of treating an immunological disorder comprising administering to a patient having or at risk of the disorder an effective regime of an antibody or fusion protein or trimeric or multimeric complex thereof as defined above.

The invention further provides a method of producing multimeric complexes of antibodies and/or fusion proteins, comprising (a) transfecting a cell with a vector or vectors encoding the first and second heavy chains as defined above, wherein antibody or fusion proteins units are expressed and assembled into a multimeric complexes via association of the multimerizing peptides on multiple units; and (b) isolating the multimeric complexes of antibodies and/or fusion proteins from the cell culture. Optionally, the first and second heavy chains are encoded by different vectors. Optionally, the multimeric complexes are trimeric complexes and the multimerizing peptides are trimerizing peptides.

### BRIEF DESCRIPTION OF THE DRAWINGS

FIG. 1: Schematic structure of antibody expression vectors.

FIGS. 2A-C: Schematic structure of monomeric and trimeric antibodies.

FIGS. 3A-C: Elution pattern of size markers (A) and HuYON007 antibodies (B and C) from a Superose 6 gel filtration column.

FIG. 4: Apoptosis of Ramos cells by monomeric (HuYON007-KH) and trimeric (HuYON007-THB) anti-DR4 IgG1 antibodies.

FIG. 5: Schematic structure of expression vectors for scFv antibodies.

FIGS. 6A, B: Sequences of IgG heavy chain constant regions.

FIG. 7A-C: Elution pattern of size markers (A) and HuOHX10 antibodies (B and C) from a Superose 6 gel filtration column.

FIG. 8: Expression of CD95 on Ramos cells

FIG. 9: Elution pattern of HuYON007 dimers from a Superose 6 gel filtration column.

### DEFINITIONS

Antibodies or fusion proteins are typically provided in isolated form. This means that an antibody or fusion protein is typically at least 50% w/w pure of interfering proteins and other contaminants arising from its production or purification but does not exclude the possibility that the monoclonal antibody or fusion protein is combined with an excess of pharmaceutical acceptable carrier(s) or other vehicle intended to facilitate its use. Sometimes antibodies or fusion proteins are at least 60, 70, 80, 90, 95 or 99% w/w pure of interfering proteins and contaminants from production or purification. Often an antibody or fusion protein is the predominant macromolecular species remaining after its purification.

Specific binding of an antibody or fusion protein to its target antigen means an affinity of at least 10⁶, 10⁷, 10⁸, 10⁹, or 10¹⁰ M⁻¹. Specific binding is detectably higher in magnitude and distinguishable from non-specific binding occurring to at least one unrelated target. Specific binding can be the result of formation of bonds between particular functional groups or particular spatial fit (e.g., lock and key type) whereas nonspecific binding is usually the result of van der Waals forces. Specific binding does not however necessarily imply that an antibody or fusion protein binds one and only one target.

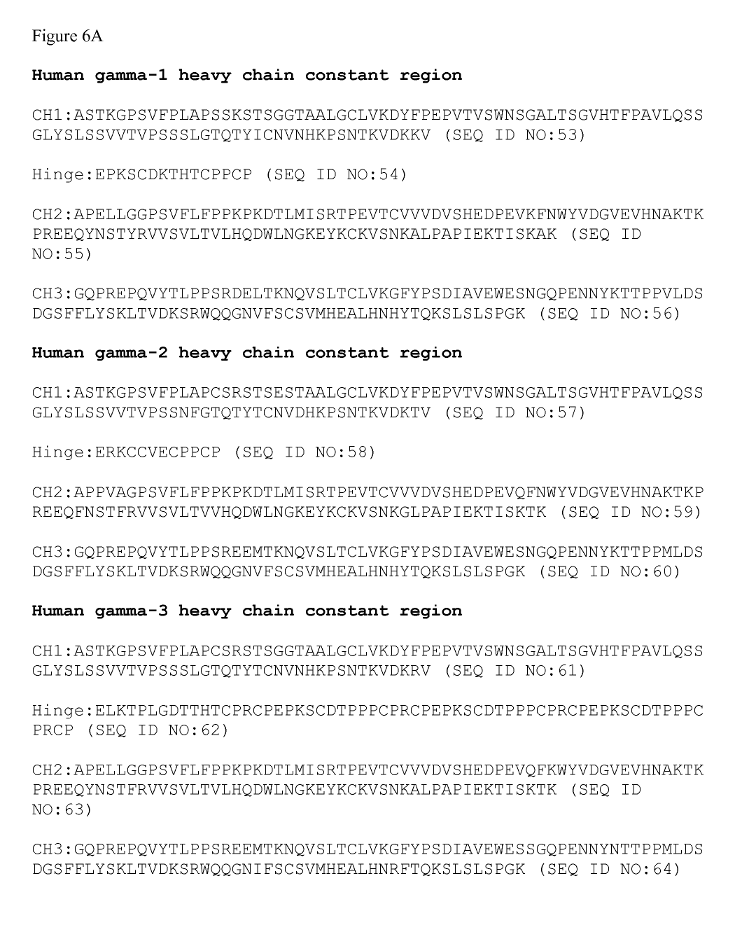

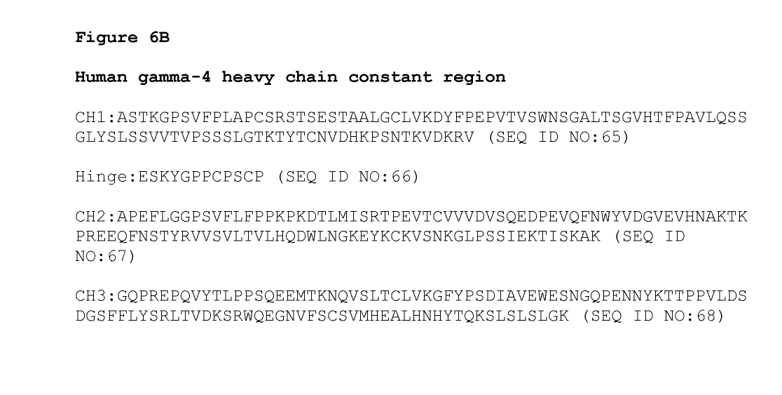

A basic antibody structural unit is a tetramer of subunits. Each tetramer includes two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. This variable region is initially expressed linked to a cleavable signal peptide. The variable region without the signal peptide is sometimes referred to as a mature variable region. Thus, for example, a light chain mature variable region means a light chain variable region without the light chain signal peptide. However, reference to a variable region does not mean that a signal sequence is necessarily present; and in fact signal sequences are cleaved once the antibodies or fusion proteins of the invention have been expressed and secreted. A pair of heavy and light chain variable regions defines a binding region of an antibody. The carboxy-terminal portion of the light and heavy chains respectively defines light and heavy chain constant regions. The heavy chain constant region is primarily responsible for effector function. In IgG antibodies, the heavy chain constant region is divided into CH1, hinge, CH2, and CH3 regions. FIGS. 6A, B show exemplary IgG sequences. The CH1 region binds to the light chain constant region by disulfide and noncovalent bonding. The hinge region provides flexibility between the binding and effector regions of an antibody and also provides sites for intermolecular disulfide bonding between the two heavy chain constant regions in a tetramer subunit. The CH2 and CH3 regions are the primary site of effector functions and FcRn binding. Light chains are classified as either kappa or lambda. Heavy chains are classified as gamma, mu, alpha, delta, or epsilon, and define the antibody's isotype as IgG, IgM, IgA, IgD and IgE, respectively. Within light and heavy chains, the variable and constant regions are joined by a “J” segment of about 12 or more amino acids, with the heavy chain also including a “D” segment of about 10 or more amino acids. (See generally, Fundamental Immunology (Paul, W., ed., 2nd ed. Raven Press, N.Y., 1989), Ch. 7) (incorporated by reference in its entirety for all purposes).

The mature variable regions of each light/heavy chain pair form the antibody binding site. Thus, an intact antibody has two binding sites, i.e., is bivalent. In natural antibodies, the binding sites are the same. However, bispecific antibodies can be made in which the two binding sites are different (see, e.g., Songsivilai and Lachmann, Clin. Exp. Immunol., 79:315-321 (1990); Kostelny et al., J. Immunol., 148:1547-53 (1992)). The variable regions all exhibit the same general structure of relatively conserved framework regions (FR) joined by three hypervariable regions, also called complementarity determining regions or CDRs. The CDRs from the two chains of each pair are aligned by the framework regions, enabling binding to a specific epitope. From N-terminal to C-terminal, both light and heavy chains comprise the domains FR1, CDR1, FR2, CDR2, FR3, CDR3 and FR4. The assignment of amino acids to each domain is in accordance with the definitions of Kabat, Sequences of Proteins of Immunological Interest (National Institutes of Health, Bethesda, Md., 1987 and 1991), or Chothia & Lesk, J. Mol. Biol. 196:901-917 (1987); Chothia et al., Nature 342:878-883 (1989). Kabat also provides a widely used numbering convention (Kabat numbering) in which corresponding residues between different heavy chain variable regions or between different light chain variable regions are assigned the same number. Although Kabat numbering can be used for antibody constant regions, the EU index is more commonly used, as is the case in this application.

An antibody or fusion protein unit, also known as a multimerization unit, is the monomeric unit of an antibody or fusion protein incorporating a homomultimerizing peptide. A multimerization unit is itself typically bivalent. In a mono-specific bivalent antibody unit, the two heavy chain and two light chain variable regions are the same. In a bispecific bivalent antibody unit, there are two different heavy and light chain variable region pairings with different binding specificities. A fusion protein unit can be homodimeric containing two copies of the same heterologous protein linked to constant regions or heterodimeric, containing two different heterologous proteins linked to constant regions.

Multimerization means the association of at least two multimerization units and more typically three, four, five or six such units via association of a homomultimerizing peptide. Valency refers to the number of binding regions or in other words, maximum number of molecules of a target antigen that can be bound by an antibody or fusion protein. A normal homodimeric IgG antibody has a valency of two. Antibodies or fusion proteins of the present invention in which the monomeric unit is bivalent, can have valencies of 6 for trimeric complexes, 8 for tetrameric complexed or 10 for pentameric complexes, 12 for hexameric complexes and so forth. These valencies are theoretical maxima. In practice, the numbers of copies of an antigen bound may be less than the theoretical maximum due to steric constraints.

An antibody or fusion protein of the invention is mono-specific if all of its antigen (or ligand) binding regions have the same specificity. An antibody or fusion protein is multispecific if its antigen binding regions include at least two different specificities. The number of different specificities in a multispecific antibody or fusion protein is typically two.

The term “antibody” includes any form of antibody with at least one binding region including monovalent fragments, bivalent tetrameric units of two heavy chains and light chains, and higher order complexes, particularly trimers, tetramers and pentamers of bivalent units. An antibody can be mono-specific in which case all binding regions have the same specificity or multi-specific in which the binding sites have at least two specificities. Likewise, a fusion protein includes a monomeric or dimeric fusion protein unit, or higher order complexes, particularly trimers, tetramers, or pentamers.

The term “epitope” refers to a site on an antigen to which an antibody or fusion protein binds. An epitope can be formed from contiguous amino acids or noncontiguous amino acids juxtaposed by tertiary folding of one or more proteins. Epitopes formed from contiguous amino acids (also known as linear epitopes) are typically retained on exposure to denaturing solvents whereas epitopes formed by tertiary folding (also known as conformational epitopes) are typically lost on treatment with denaturing solvents. Some antibodies bind to an end-specific epitope, meaning an antibody binds preferentially to a polypeptide with a free end relative to the same polypeptide fused to another polypeptide resulting in loss of the free end. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols, in Methods in Molecular Biology, Vol. 66, Glenn E. Morris, Ed. (1996).

The term “antigen” or “target antigen” indicates a target molecule bound by an antibody or fusion protein. An antigen may be a protein of any length (natural, synthetic or recombinantly expressed), a nucleic acid or carbohydrate among other molecules. Antigens include receptors, ligands, counter receptors, and coat proteins.

A heterologous polypeptide in a fusion protein is a polypeptide not naturally linked to an immunoglobulin constant region. Such a polypeptide can be a full-length protein or any fragment thereof of sufficient length to retain specific binding to the antigen bound by the full-length protein. For example, a heterologous polypeptide can be a receptor extracellular domain or ligand thereto.

Antibodies that recognize the same or overlapping epitopes can be identified in a simple immunoassay showing the ability of one antibody to compete with the binding of another antibody to a target antigen. The epitope of an antibody can also be defined X-ray crystallography of the antibody bound to its antigen to identify contact residues. Alternatively, two antibodies have the same epitope if all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

Competition between antibodies is determined by an assay in which an antibody under test inhibits specific binding of a reference antibody to a common antigen (see, e.g., Junghans et al., Cancer Res. 50:1495, 1990). A test antibody competes with a reference antibody if an excess of a test antibody (e.g., at least 2×, 5×, 10×, 20× or 100×) inhibits binding of the reference antibody by at least 50% but preferably 75%, 90% or 99% as measured in a competitive binding assay. Antibodies identified by competition assay (competing antibodies) include antibodies binding to the same epitope as the reference antibody and antibodies binding to an adjacent epitope sufficiently proximal to the epitope bound by the reference antibody for steric hindrance to occur.

The term “patient” includes human and other mammalian subjects that receive either prophylactic or therapeutic treatment.

For purposes of classifying amino acids substitutions as conservative or nonconservative, amino acids are grouped as follows: Group I (hydrophobic side chains): met, ala, val, leu, ile; Group II (neutral hydrophilic side chains): cys, ser, thr; Group III (acidic side chains): asp, glu; Group IV (basic side chains): asn, gln, his, lys, arg; Group V (residues influencing chain orientation): gly, pro; and Group VI (aromatic side chains): trp, tyr, phe. Conservative substitutions involve substitutions between amino acids in the same class. Non-conservative substitutions constitute exchanging a member of one of these classes for a member of another.

Percentage sequence identities are determined with antibody sequences maximally aligned by the Kabat numbering convention for a variable region or EU numbering for a constant region. For other proteins, sequence identity can be determined by aligning sequences using algorithms, such as BESTFIT, FASTA, and TFASTA in the Wisconsin Genetics Software Package Release 7.0, Genetics Computer Group, 575 Science Dr., Madison, Wis.), using default gap parameters, or by inspection, and the best alignment. After alignment, if a subject antibody region (e.g., the entire mature variable region of a heavy or light chain) is being compared with the same region of a reference antibody, the percentage sequence identity between the subject and reference antibody regions is the number of positions occupied by the same amino acid in both the subject and reference antibody region divided by the total number of aligned positions of the two regions, with gaps not counted, multiplied by 100 to convert to percentage.

Compositions or methods “comprising” one or more recited elements may include other elements not specifically recited. For example, a composition that comprises antibody may contain the antibody alone or in combination with other ingredients.

The term “antibody-dependent cellular cytotoxicity,” or ADCC, is a mechanism for inducing cell death that depends upon the interaction of antibody-coated target cells (i.e., cells with bound antibody) with immune cells possessing lytic activity (also referred to as effector cells). Such effector cells include natural killer cells, monocytes/macrophages and neutrophils. ADCC is triggered by interactions between the Fc region of an antibody bound to a cell and Fcγ receptors, particularly FcγRI and FcγRIII, on immune effector cells such as neutrophils, macrophages and natural killer cells. The target cell is eliminated by phagocytosis or lysis, depending on the type of mediating effector cell. Death of the antibody-coated target cell occurs as a result of effector cell activity.

The term opsonization also known as “antibody-dependent cellular phagocytosis,” or ADCP, refers to the process by which antibody-coated cells are internalized, either in whole or in part, by phagocytic immune cells (e.g., macrophages, neutrophils and dendritic cells) that bind to an immunoglobulin Fc region.

The term “complement-dependent cytotoxicity” or CDC refers to a mechanism for inducing cell death in which an Fc effector domain(s) of a target-bound antibody activates a series of enzymatic reactions culminating in the formation of holes in the target cell membrane. Typically, antigen-antibody complexes such as those on antibody-coated target cells bind and activate complement component Clq which in turn activates the complement cascade leading to target cell death. Activation of complement may also result in deposition of complement components on the target cell surface that facilitate ADCC by binding complement receptors (e.g., CR3) on leukocytes.

pH-dependent binding of an antibody to an FcRn receptor means that an antibody binds more strongly to such a receptor at pH 6.0 than at pH 7.5. Binding of FcRn at a low pH in endosomes after internalization by pinocytosis rescues IgG antibodies from catabolic degradation in lysosomes. Rescued IgG antibodies are then released from FcRn at a neutral pH and recycled to the circulation. Such pH-dependent FcRn binding is the basis of the molecular mechanism for a long serum half-life of IgG antibodies (Ghetie et al., Annu. Rev. Immunol. 18:739-766, 2000). For example, human IgG antibodies bind to human neonatal Fc receptors (FcRn) at pH 6.0 while they bind only weakly to FcRn at pH 7.5. The FcRn binding site in IgG antibodies lies at the junction of the CH2 and CH3 domains. Because a μ heavy chain does not bind to FcRn at pH 6.0 or 7.5, natural IgM cannot take advantage of the FcRn-mediated pathway to rescue antibodies from degradation in lysosomes and therefore in general have shorter half-lives than natural IgG antibodies.

A humanized antibody is a genetically engineered antibody in which the CDRs from a non-human “donor” antibody are grafted into human “acceptor” antibody sequences (see, e.g., Queen, U.S. Pat. Nos. 5,530,101 and 5,585,089; Winter, U.S. Pat. No. 5,225,539, Carter, U.S. Pat. No. 6,407,213, Adair, U.S. Pat. No. 5,859,205 6,881,557, Foote, U.S. Pat. No. 6,881,557). The acceptor antibody sequences can be, for example, a mature human antibody sequence, a composite of such sequences, a consensus sequence of human antibody sequences, or a germline region sequence. Thus, a humanized antibody is an antibody having some or all CDRs entirely or substantially from a donor antibody and variable region framework sequences and constant regions, if present, entirely or substantially from human antibody sequences. Similarly a humanized heavy chain has at least one, two and usually all three CDRs entirely or substantially from a donor antibody heavy chain, and a heavy chain variable region framework sequence and heavy chain constant region, if present, substantially from human heavy chain variable region framework and constant region sequences. Similarly a humanized light chain has at least one, two and usually all three CDRs entirely or substantially from a donor antibody light chain, and a light chain variable region framework sequence and light chain constant region, if present, substantially from human light chain variable region framework and constant region sequences. Other than nanobodies and dAbs, a humanized antibody comprises a humanized heavy chain and a humanized light chain. A CDR in a humanized antibody is substantially from a corresponding CDR in a non-human antibody when at least 85%, 90%, 95% or 100% of corresponding residues (as defined by Kabat) are identical between the respective CDRs. The variable region framework sequences of an antibody chain or the constant region of an antibody chain are substantially from a human variable region framework sequence or human constant region respectively when at least 85, 90, 95 or 100% of corresponding residues defined by Kabat are identical.

Although humanized antibodies often incorporate all six CDRs (preferably as defined by Kabat) from a mouse antibody, they can also be made with less than all CDRs (e.g., at least 3, 4, or 5 CDRs from a mouse antibody) (e.g., Pascalis et al., J. Immunol. 169:3076, 2002; Vajdos et al., Journal of Molecular Biology, 320: 415-428, 2002; Iwahashi et al., Mol. Immunol. 36:1079-1091, 1999; Tamura et al, Journal of Immunology, 164:1432-1441, 2000).

A chimeric antibody is an antibody in which the mature variable regions of light and heavy chains of a non-human antibody (e.g., a mouse) are combined with human light and heavy chain constant regions. Such antibodies substantially or entirely retain the binding specificity of the mouse antibody, and are about two-thirds human sequence.

A veneered antibody is a type of humanized antibody that retains some and usually all of the CDRs and some of the non-human variable region framework residues of a non-human antibody but replaces other variable region framework residues that may contribute to B- or T-cell epitopes, for example exposed residues (Padlan, Mol. Immunol. 28:489, 1991) with residues from the corresponding positions of a human antibody sequence. The result is an antibody in which the CDRs are entirely or substantially from a non-human antibody and the variable region frameworks of the non-human antibody are made more human-like by the substitutions.

A human antibody can be isolated from a human, or otherwise result from expression of human immunoglobulin genes (e.g., in a transgenic mouse, in vitro or by phage display). Methods for producing human antibodies include the trioma method of Oestberg et al., Cys muoma 2:361-367 (1983); Oestberg, U.S. Pat. No. 4,634,664; and Engleman et al., U.S. Pat. No. 4,634,666, use of transgenic mice including human immunoglobulin genes (see, e.g., Lonberg et al., WO93/12227 (1993); U.S. Pat. No. 5,877,397, U.S. Pat. No. 5,874,299, U.S. Pat. No. 5,814,318, U.S. Pat. No. 5,789,650, U.S. Pat. No. 5,770,429, U.S. Pat. No. 5,661,016, U.S. Pat. No. 5,633,425, U.S. Pat. No. 5,625,126, U.S. Pat. No. 5,569,825, U.S. Pat. No. 5,545,806, Nature 148, 1547-1553 (1994), Nature Biotechnology 14, 826 (1996), Kucherlapati, WO 91/10741 (1991) and phage display methods (see, e.g. Dower et al., WO 91/17271 and McCafferty et al., WO 92/01047, U.S. Pat. No. 5,877,218, U.S. Pat. No. 5,871,907, U.S. Pat. No. 5,858,657, U.S. Pat. No. 5,837,242, U.S. Pat. No. 5,733,743 and U.S. Pat. No. 5,565,332.

Protein A is a 40-60 kDa surface protein originally found in the cell wall of the bacterium Staphylococcus aureus. Protein A specifically binds with high affinity to human IgG1, IgG2 and IgG4 as well as mouse IgG2a and IgG2b. It does not bind to human IgG3 or IgA, or IgM. Protein A is used for affinity purification of antibodies.

Protein G is a 65-kDa (G148 protein G) and a 58 kDa (C40 protein G) Streptococcal cell surface protein. It contains a serum albumin binding domain not needed for IgG binding, which is often deleted. Protein G specifically binds to all of the human IgG isotypes but not IgA or IgM. Protein G is also useful for antibody purification.

When an antibody of the invention (present antibody) is said to retain a property of a parental antibody from which it was derived (i.e., without modification of the heavy chain constant regions and without addition of a homomultimerizing peptide), retention can be partial or complete. Complete retention of an activity between a present antibody of the invention and a parent antibody from which it was derived means the activity of the present antibody is the same within experimental error or greater than that of the parent antibody. Partial retention of activity means that an activity of the present antibody is significantly above background level of a negative control (i.e., beyond experimental error) and preferably at least 50% of the corresponding activity of the parent antibody.

### DETAILED DESCRIPTION

### I. General

The invention provides antibodies or fusion proteins with modified heavy chain IgG constant regions that promote assembly of multimeric complexes. Within an antibody or fusion protein unit there are two heavy chains each including at least CH2 and CH3 regions. The two heavy chains can bear complementary modifications (e.g., knob and hole) to promote coupling of the heavy chains within a unit. One and only one of the heavy chains in a unit is fused at its C-terminus to a homomultimerizing peptide. The presence of the homomultimerizing peptide promotes association between units. For example, if the homomultimerizing peptide is a homotrimerizing peptide it promotes association of three units to form a trimeric complex. Such a complex typically has six binding sites, two on each unit. The binding sites can have the same or different specificities. If different, each unit of the complex typically has each of two binding specificities.

The antibodies and fusion proteins specifically bind to protein G, which facilitates purification. The antibodies and fusion proteins optionally retain completely or partially IgG properties including pH-dependent FcRn binding, which is associated with a relatively long in vivo half-life. Depending on the isotype and subtype, the nature of the antigen and presence of additional IgG CH1 and hinge domains, IgG heavy chain constant regions of the invention may also retain completely or partially properties of specific binding to protein A, and effector functions ADCC, CDC and opsonization.

The combination of IgG effector functions, relatively long half-life and ease of purification with ability to multimerize results in antibodies or fusion protein with novel combinations of properties. For example, some such antibodies or fusion protein can effectively multimerize receptors or bound ligands on the cell surface while maintaining completely or partially, or even enhancing, Fcγ-mediated properties such as ADCC, CDC, opsonization, pH-dependent FcRn binding, and the ability to bind to Protein A and Protein G relative to antibodies having an unmodified IgG isotype. The combination of properties from different isotypes offers the possibility of greater potency than conventional IgG, IgM or IgA antibodies for treatment of cancer and other diseases.

The above advantages can be achieved without in vitro manipulations other than those involved in making nucleic acid constructs for expression of the antibodies or fusion proteins incorporating the modified forms of heavy chain constant regions.

### II. Components of Constant Regions

The heavy chain constant regions include an IgG portion including at least IgG CH2 and CH3 regions. One and only of the constant regions within an antibody or fusion protein unit is fused to a homomultimerizing peptide at its C-terminus. The two heavy chain constant regions can include complementary mutations to promote their association. The position chosen for mutation should support intermolecular association between heavy chains of antibody or fusion protein units, preferably without substantial impairment of desired effector functions. The CH2 and CH3 regions are responsible at least in part for FcRn binding, protein A and G binding, ADCC, CDC and opsonization. The IgG portion also preferably includes a hinge region and/or a CH1 region. The hinge region provides flexibility between the binding region and effector region of an antibody or fusion protein and contributes to efficient effector functions, such as ADCC, opsonization and CDC. The hinge region is also the site of disulfide bonds that link a pair of IgG heavy chains together. The CH1 region bonds with a light chain constant region and is generally included in formats in which a light chain with light chain constant region is present but can be omitted in fusion proteins or single-chain antibody formats in which no light chain constant region is present. The CH1 region can be replaced by a light chain constant region in “crossing over” formats discussed below.

The components mentioned above are arranged from N-terminus to C-terminus in the order: IgG CH1 region (if present), IgG hinge region (if present), IgG CH2 region, IgG CH3 region, homomultimerizing peptide (in the chain in which it is present).

Usually, all of the IgG regions are of the same isotype and subtype. For example, all IgG regions are either from IgG1, IgG2, IgG3 or IgG4.

Preferably, the IgG regions are human IgG. Exemplary sequences for human IgG1, IgG2, IgG3, and IgG4 heavy chains with delineation into components (CH1, hinge, CH2, CH3), are shown in FIGS. 6 A, B. However, regions from other species including nonhuman primates, camelids, cartilaginous fish, mice or rats can also be used.

Reference to a human IgG region (i.e., CH1, hinge, CH2, CH3) refers to the exemplified sequences or allotypes or isoallotypes thereof or other variant sequence having at least 90, 95, 98 or 99% sequence identity with an exemplified sequence and/or differing from the exemplified sequence by up to 1, 2, 3, 4, 5, 10 or 15 amino acid deletions, substitution or internal insertions in the case of CH1, CH2, CH3, and 1, 2 or 3 deletions, substitutions or internal substitutions for IgG1, 2 or 4 hinge regions and up to 1, 2, 3, 4, 5, or 6 deletions, substitutions or internal substitutions for IgG3 hinge. Substitutions, if present, are preferably conservative. Human constant regions show allotypic variation and isoallotypic variation between different individuals, that is, the constant regions can differ in different individuals at one or more polymorphic positions. Isoallotypes differ from allotypes in that sera recognizing an isoallotype bind to a non-polymorphic region of a one or more other isotypes. Reference to a human constant region includes a constant region with any natural allotype (including isoallotypes) or any permutation of residues occupying polymorphic positions in natural allotypes. Sequences of non-human constant regions are provided by e.g., the Swiss-Prot or Genbank databases. Reference to a non-human constant region likewise includes allotypic or isoallotypic variants, and permutations of the same, or other variants sequences differing from natural sequences. The scope of variations is defined by sequence identity and/or number of substitutions with respect to natural sequences of non-human constant regions in analogous fashion to the above description of variants with respect to human constant regions. The Eu numbering convention is used in defining corresponding positions among isotypes or different species, or defining mutated positions.

One or several amino acids at the amino or carboxy terminus of the light and/or heavy chain, such as a C-terminal lysine of the heavy chain, may be missing or derivatized in a proportion or all of the molecules. Substitutions can be made in the constant regions to reduce or increase effector function such as complement-mediated cytotoxicity or ADCC (see, e.g., Winter et al., U.S. Pat. No. 5,624,821; Tso et al., U.S. Pat. No. 5,834,597; and Lazar et al., Proc. Natl. Acad. Sci. USA 103:4005, 2006), or to prolong half-life in humans (see, e.g., Hinton et al., J. Biol. Chem. 279:6213, 2004). Exemplary substitutions include a Gln at position 250 and/or a Leu at position 428 (EU numbering) for increasing the half-life of an antibody. Substitution at any of positions 234, 235, 236 and/or 237 reduces affinity for Fcγ receptors, particularly FcγRI receptor (see, e.g., U.S. Pat. No. 6,624,821). Optionally, positions 234, 236 and/or 237 in human IgG2 are substituted with alanine and position 235 with glutamine. (See, e.g., U.S. Pat. No. 5,624,821.)

If a hinge region is used, part of the hinge can be replaced by a synthetic linker molecule. Such is often the case in fusion proteins in which a binding region of the fusion protein is joined to CH2 and CH3 IgG or IgA constant regions via a hinge region in which, for example, up to 10 N-terminal residues are replaced by a synthetic flexible linker. Gly-Gly-Ala-Ala, Gly-Gly-Gly-Gly-Ser, Leu-Ala-Ala-Ala-Ala and multimers thereof are examples of such a linker. The hinge region can also be replaced in its entirety by a synthetic linker or omitted without replacement.

With the possible exception of a synthetic linker replacing part or all of a hinge region and one or a few amino acid substitutions to enhance or suppress effector functions or FcRn binding as discussed further below, 1-4 mutations per chain to promote association and linkage of one chain to a homomultimerizing peptide at its C-terminus, it is preferred that constant regions contain no sequences other than the CH1, hinge, CH2, CH3, regions mentioned above. Nevertheless, other sequences, such as for example, a hexa-histidine tag, can be added but are not necessary.

### III. Multimerizing Peptides

The invention employs homo multimerizing (sometimes abbreviated to “multimerizing”) peptides that assemble into a homomultimer alone and when linked to a heavy chain constant region of the invention. The peptides can be (but need not be) of relatively short length (e.g., up to 50 or 100 amino acids).

A peptide with homodimerizing ability is a leucine zipper, which is a common three-dimensional structural motif in proteins. These motifs are usually found as part of a DNA-binding domain in various transcription factors. A single leucine zipper includes multiple leucine residues at approximately 7-residue intervals, which forms an amphipathic alpha helix with a hydrophobic region running along one side. SEQ ID NO:42 provides an example of a leucine zipper. Other examples of peptides with homodimerizing ability are reported by Jones (Genome Biol. 5:226, 2004), Woolfson (Adv. Protein Chem. 70:79-112, 2005), Parry et al. (J. Struc. Biol. 163:258-69, 2008), Zaccai et al. (Nat. Chem. Biol. 7:935-941, 2011), and Ivarsson (FEBS Lett. 586:2638-2647, 2012).

Known trimerizing peptides (i.e., peptides forming homotrimers) include an isoleucine zipper, which is a peptide having an amino acid sequence with an overrepresentation of isoleucine residues (compared with human proteins in general) and the ability to form a homotrimer. Several examples of isoleucine zipper sequences in humans and other species are provided in the Swiss Prot database (e.g., Q86TE4, Q86V48). An isoleucine zipper peptide used in the present examples has the sequence MKQIEDKIEEILSKIYHIENEIARIKKLIGERAG (SEQ ID NO:12). Variants of this sequence or other known sequences in the Swiss Prot database having at least 90 or 95% sequence identity thereto or functional fragments or peptides comprising designated sequences (i.e., with additional flanking regions) can be used provided such variants retain trimerizing ability.

Another peptide with trimerizing ability is tetranectin. An exemplary form of human tetranectin is provided by Swis Prot. P05452. Reference to tetranectin refers peptides having an amino acid sequence consisting of or comprising to this sequence, species homologs (several of which are know), allelic variants (several of which are described in the Swiss-Prot database), other sequences having a least 90% or 95% sequence identity to P05442 and, or functional fragments of P05442. Such variants should retain trimerizing ability. Other trimerizing peptides include peptides consisting of or comprising the extracellular domains of TNF superfamily members. Examples of TNF superfamily members include human TNF (Swiss Prot P01375), human CD40 ligand (P29965), and OX40-L (P23510).

A preferred trimerizing peptide is the extracellular domain of TNF (Swiss Prot P01375) which has the sequence VRSSSRTPSDKPVAHVVANPQAEGQLQWLNRRANALLANGVELRDNQLVVPSEGLYLISQVLFKGQGCPS THVLLTHTISRIAVSSQTKVNLLSAIKSPCQRETPEGAEAKPWYEPIYLGGVFQLEKGDRLSAEINRPDYLDFAES GQVYFGIIAL (SEQ ID NO:13). Variants of TNF superfamily member extracellular domains having at least 90 or 95% identity in amino acid sequence to a natural human TNF superfamily member and functional fragments can be used provided the variant retains the desired trimerizing ability.

The strategy and principles for making trimeric complexes of antibodies or fusion proteins can be extended to higher order multimers by replacing the trimerizing peptide with a multimerizing peptide that associates to a homomultimer of the desired number of units. Examples of tetramerizing peptides for making tetrameric complexes of an antibody or fusion protein unit are tetrabrachion (Stetefeld et al., Naure Struc. Biol. 7:772-776, 2000), modified GCN4 leucine zipper (Harbury et al., Science 262:1401-1407, 1993), and Sendai virus phosphoprotein (Tarbouriech et al., Nature Struc. Biol. 7:777-781, 2000). For forming pentameric IgG antibodies and Fc fusion proteins, a pentamerizing peptide, for example, Trp-zipper protein (also called Trp-14; Liu et al., Proc. Natl. Acad. Sci. USA 101:16156-16161. 2004) or cartilage oligomeric matrix protein (COMP; Malashkevich et al., Science 274: 761-765, 1996) can be used. For forming hexameric IgG antibodies and fusion proteins, a hexamerizing peptide, such as CC-Hex (Zaccai et al., Nature Chem. Biol. 7:935-941, 2011) can be used. Variants of any of the disclosed tetramerizing, pentamerizing or hexamerizing peptides consisting or comprising of the disclosed peptides, having at least 90 or 95% sequence identity thereto at the amino acid level, or functional fragments thereof can be used provided the variant retains the desired multimerizing ability.

### IV. Modifications of Heavy Chain Constant Regions

Pairing of the two different heavy chains of the invention (i.e. with and without the multimerizing peptide) is achieved by introducing complementary modifications of natural IgG sequences that favor association of the different chain as a heterodimer over homodimeric pairing of the same heavy chain constant region. One such modification is the introduction of a knob in one heavy chain and a hole in the other heavy chain such that coupling of the knob to the hole promotes the desired heterodimer formation. Knobs and holes are terms of art in the antibody field. A knob refers to the replacement of one or a few (e.g., up to 4) contiguous or otherwise spatially proximate amino acids with larger amino acids (by molecular weight). Conversely, a hole refers to the replacement of one or a few (e.g., up to 4) contiguous or otherwise spatially proximate amino acids with smaller amino acids. Knobs and holes are usually inserted into the C_{H}3 regions of the respective heavy chains (Ridgway et al., Protein Eng 9, 617-21 (1996); Atwell et al., J. Mol. Biol. 270, 26-35 (1997)). The amino acid introduced to increase or decrease size and create a knob or hole is preferably a conservative substation. A preferred modification to create a hole in human IgG1 is a combination of T366S, L368A and Y407V mutations (natural amino acid first, location by Eu numbering second, mutated amino acid third). A preferred modification to create a knob in human IgG1 is a T366W mutation. Other known knob:hole pairs are T366Y:Y407T and T366W:Y407A.

Heterodimeric Fc-to-Fc interaction of IgG antibodies can also be achieved by changing the charge complementarity at the interface. An example of such modification is a double mutation (E356K+D399K) in a human IgG Fc, which adds positive charge at the interface, and a double mutation (K362D+K409D) in another human IgG Fc, which adds negative charge at the interface (Gunasekaran et al., J. Biol. Chem. 285: 19637-19646, 2010; Liu et al., J. Biol. Chem. 289:3571-3590, 2014). Other Fc mutations that promote Fc-to-Fc heterodimer formation were reported by Choi et al. (Mol. Cancer. 12:2748-2759, 2013) and Moore et al. (MABs 3:546-557, 2011)

When one amino acid is said to replace another what is meant is that the amino acids occupy corresponding positions in two variants of a protein. In the context of antibodies, corresponding positions are determined by the Kabat numbering system for the variable regions and EU index for the C_{H} region. Whether an introduced amino acid is larger or smaller than a replaced amino acid can be determined with reference to a natural heavy chain constant region sequence, such as any of the human IgG1, IgG2, IgG3 or IgG4 sequences.

### IV. Properties of Antibodies and Fusion Proteins Incorporating Modifications of the Invention

The properties of an antibody or fusion protein incorporating heavy chain constant regions as described above depend in part on the isotype, and subtype of the CH1, hinge (if present), CH2 and CH3 regions, whether the CH1 and/or hinge regions are present, and the nature of the antigen bound by the antibody or fusion protein.

Antibodies and fusion proteins incorporating the constant regions of the invention retain at least the ability to multimerize a monovalent or bivalent unit to higher valency and at least one property of IgG antibodies. When CH1, hinge (if present), CH2 and CH3 are of IgG origin, the antibodies completely or partially retain at least the IgG-like properties of binding protein G, as well as capacity to specifically bind to a target antigen. pH-dependent FcRn binding may also be partially or completely retained.

Selection of isotype or subtype depends on the desired properties. IgG1 or IgG3 is selected if strong effector functions are desired (as is often the case against cancer cells, pathogens) and IgG2 or IgG4 is selected if weaker or no CDC, ADCC and opsonization are required (as may be the case if the mechanism is inhibition of a receptor-ligand interaction).

When the CH1 and hinge regions (if present), CH2 and CH3 regions are human IgG1, then an antibody or fusion protein incorporating a heavy chain constant region of the invention has specific binding to protein A and protein G, and may have pH-dependent FcRn binding and effector functions, such as ADCC, CDC, opsonization depending on the antigen bound. Such effector functions are usually present if the antigen bound is a surface receptor (e.g., on a cell or virus). If the antigen is normally in soluble form, effector functions are not usually expressed against the soluble antigen but can be demonstrated by expressing the antigen in bound form (e.g., on a cell surface).

When the CH1 and hinge regions (if present), CH2 and CH3 regions are human IgG2, IgG4, then an antibody or fusion protein incorporating a heavy chain constant region of the invention shows at least specific binding to protein A and protein G, and may have pH-dependent FcRn binding. Human IgG2 and IgG4 isotypes generally lack CDC. IgG4 has some ADCC and opsonization against bound antigens but less than human IgG1 or IgG3.

When the CH1 and hinge regions (if present), CH2 and CH3 regions are human IgG3, then an antibody or fusion protein incorporating a heavy chain constant region of the invention shows at least specific binding to protein G, and may have pH-dependent FcRn binding. Such an antibody or fusion protein may also show effector functions, such as ADCC, CDC, opsonization depending on whether the antigen bound is a surface antigen or soluble, as is the case for IgG1.

In antibodies or fusion proteins with constant regions of the invention in which CDC, ADCC or opsonization is present, the level of CDC, ADCC, or opsonization is sometimes the same as (within experimental error) or sometimes greater than that of an otherwise comparable antibody or fusion protein with a conventional IgG constant region.

### V. Antibody and Fusion Protein Formats

Heavy chain constant regions of the invention can be incorporated into mono or bispecific antibodies or fusion proteins, which can assemble in multimeric forms. For expression of a mono-specific antibody, the same heavy chain variable region is expressed from two expression units linked to the two different constant regions of the invention. A light chain is expressed comprising a variable region and constant region. Each of the heavy chains binds to the light chain via the CH1 region of the heavy chain and light chain constant region of the light chain (or vice versa in cross-over formats) to a form a heterodimer. Two heterodimers then pair by association of hinge, CH2 and CH3 regions of the IgG portion of the heavy chain to form a tetramer unit, as is the case for a conventional antibody. However, the association of heterodimers with different constant regions over the same constant region is favored by the presence of complementary modifications in the different heavy chain constant regions (e.g., knob and hole) promoting their mutual association. Thus, tetramers preferably associate including two different heavy chain constant regions, only one of which has a linked multimerizing peptide. Tetramer units then multimerize via association of the multimerizing peptide.

The heavy chain constant regions can be used with any type of engineered antibody including chimeric, humanized, veneered or human antibodies. The antibody can be a monoclonal antibody or a genetically engineered polyclonal antibody preparation (see U.S. Pat. No. 6,986,986).

For a monospecific fusion protein, the heavy chain constant regions of the invention are expressed, each linked to the same heterologous polypeptide. The heterologous polypeptide provides a binding region at the N-terminus of the constant region and is sometimes referred to simply as a binding region. The IgG CH1 region is not typically included in the constant region for fusion proteins. The IgG hinge region may or may not be included. In some fusion proteins, part or all of the hinge region is replaced by a synthetic linker peptide conferring flexibility between the binding portion of a fusion protein and heavy chain constant region.

The binding region of a fusion protein can be any of the types of binding portion used in other fusion proteins produced to date (among others). Examples of binding regions are extracellular domains of cellular receptors or their ligands or counter-receptors (e.g., TNF-alpha receptor, death family receptor, LFA3 or IL-1 receptor or Trail).

Both antibody and fusion proteins can be expressed in a multi-specific (typically bi-specific) format, that is, as a complex containing antibody or fusion protein units within different target specificities. For antibodies, this is achieved by fusing two different variable regions to the two different heavy chain constant regions. For example, the different variable regions may have specificity to different targets. The light chain variable regions can be the same or different. If the light chains are different, correct pairing of the light and heavy chains to form each heterodimer can be promoted by “crossing over” of heavy chain and light chain domains within one of the heterodimers (Schaefer et al., Proc Natl Acad Sci USA 108:11187-92, 2011; WO 2009/080251; WO 2009/080252; WO 2009/080253).

In some bispecific antibodies with two different heavy chain variable regions and two different light chain variable regions, one heavy chain variable region and one light chain variable region come from one parental antibody, and the other antibody heavy chain variable region and light chain variable region come from another parental antibody. Such expression results in an antibody unit having the two specificities for example, to two different targets. When such an antibody unit multimerizes, each of the units in the resulting multimeric complex includes both specificities. Higher multi-specificities can be obtained by expressing additional heavy chain and light chain variable regions linked to the same constant regions from separate expression units. For example, for expression of a complex with four binding specificities, two different heavy chain variable regions can be expressed linked to one heavy chain constant region of the invention (from separate expression units) and two further different heavy chain variable regions can be expressed linked to the other heavy chain constant region of the invention (again from separate expression units). Up to four light chain variable regions linked to a light chain constant region (or CH1 region in cross-over formats) can also be expressed from separate units. Multi-specificity complexes assemble including four binding specificities, albeit not necessarily in equal proportions in any complex.

Fusion proteins can likewise be expressed in multi-specific format by fusing two different heterologous polypeptides to the two constant regions of the invention. Units of a multispecific fusion protein then contain each of the different heterologous polypeptides. Higher multi-specificities can be obtained by expressing further heterologous polypeptides from separate expression units linked to one or both of the constant regions of the invention.

A hybrid of an antibody and fusion protein can also be formed. In this case, one heavy chain constant region of the invention is fused to an antibody heavy chain variable region and expressed with a light chain including a constant regions and variable region. The other heavy chain constant of the invention is fused to a heterologous polypeptide. The resulting hybrid antibody fusion protein unit has two binding specificities, one conferred by a heavy light chain pair, the other by the heterologous polypeptide, the different binding specificities held together by association of the different heavy chains. Such a hybrid unit can multimerize via a multimerizing peptide as can antibody or fusion protein units.

A multi-specific antibody or fusion protein can include binding specificities for an antigen on a target (e.g., a cancer cell or pathogen) and for an antigen on an effector cell (e.g., CD3 on a T-cell). Such a multi-specific complex forms a bridge between the target cell and effector cell and promotes cytotoxic or opsonization activity of the effector cell. A multi-specific antibody or fusion protein can additionally or alternatively include binding specificities for two different antigens on the same target (e.g., a cancer cell or pathogen). Such an antibody or fusion protein can have greater selective toxicity to the target cell than an antibody or fusion protein with specificity for a single antigen. Other multi-specific antibodies or fusion proteins include binding regions for both a receptor and its ligand or counter-receptor. Such antibodies or fusion proteins can exert greater inhibition than antibodies or fusion proteins binding receptor or ligand/counterreceptor alone. Any of these specificities and others can be combined in the same multi-specific complex.

### VI. Genetic Engineering and Expression

Antibodies or fusion proteins including the modifications described above are produced by recombinant expression. Production of an antibody typically requires several expression units, one for each for the different heavy chains, and one or two for the two light chains depending whether the light chains are the same or different. The expression units can be present on separate vectors, or split among two or more vectors, or all can be present on the same vector. Production of an Fc fusion protein typically requires two expression units, one for each heavy chain. The expression units can be on the same or different vectors. One heavy chain expression vector expresses a heavy chain contain region fused at the C-terminus to the multimerizing peptide and at the N-terminus to a heavy chain variable region or heterologous polypeptide in turn fused to a signal peptide. The other heavy chain expression vector expresses the other heavy chain constant region (without multimerizing peptide), again fused at its N-terminus to a heavy chain variable region or heterologous polypeptide, in turn fused to a signal sequence. The heavy chain expression units bear different modification of natural IgG sequences to promote association. Such modification can be introduced by methods, such as site specific or cassette mutagenesis or introduced in de novo nucleic acid synthesis. The light chain expression units (for antibody production) include from N-terminus to C-terminus a signal peptide, a variable region and a light chain constant region, as for standard expression of an antibody.

The order in which fusions of genetic elements is performed in building a construct encoding several components is not important. For example, a DNA segment encoding a heavy chain variable region can be linked to DNA encoding an IgG heavy chain constant region, which can in turn linked to DNA encoding a multimerizing peptide, or the segments encoding a heavy chain constant region and multimerizing peptide can be linked to one another first. The segments can also be linked simultaneously by joining overlapping oligonucleotides encoding the respective segments in an overlapping PCR-type reaction. In practice, once expression units encoding the heavy chain constant regions of the invention have been produced, the same expression units can be used to insert any heavy chain variable region(s) or other binding region(s) in the case of a fusion protein (and sometimes a light chain variable region) without recreating the DNA segment encoding all of the heavy chain components.

Mammalian cells are a preferred host for expressing nucleotide segments encoding antibodies or fusion proteins of the invention (see Winnacker, From Genes to Clones, (VCH Publishers, NY, 1987)). A number of suitable host cell lines capable of secreting intact heterologous proteins have been developed in the art, and include CHO cell lines, various COS cell lines, HeLa cells, HEK293 cells, L cells, and non-antibody-producing myelomas including Sp2/0 and NS0. Preferably, the cells are nonhuman. Preferably, an antibody or fusion protein of the invention is expressed from a monoclonal cell line.

Expression vectors for these cells can include expression control sequences, such as an origin of replication, a promoter, an enhancer (Queen et al., Immunol. Rev. 89:49 (1986)), and necessary processing information sites, such as ribosome binding sites, RNA splice sites, polyadenylation sites, and transcriptional terminator sequences. Preferred expression control sequences are promoters derived from endogenous genes, cytomegalovirus, SV40, adenovirus, bovine papillomavirus, and the like. See Co et al., J. Immunol. 148:1149 (1992).

Cells are transfected with one or more vectors encoding the antibody or fusion protein to be expressed. For a multi-chain antibody, the heavy and light chains can be expressed on the same or separate vectors. For expression of multi-specific complexes, the DNA encoding the components of the complexes (i.e., different antibodies or fusion proteins) can be on the same or different vectors.

Antibody or fusion protein chains are expressed, processed to remove signal peptides, assembled and secreted from host cells. It is believed that association of different heavy chains, association between heavy and light chains (in the case of antibody) and multimerization of antibody or fusion protein units occur at least predominantly within cells so that antibodies or fusion proteins are secreted primarily as multimers, particularly trimers when a trimerizing peptide is used (or tetramers or pentamers if a tetramizing or pentamerizing peptide is used).

Antibodies or fusion proteins can be purified from cell culture supernatants by conventional antibody purification methods. The purification can include a chromatography step using protein A or protein G as the affinity reagent. Conventional antibody purification procedures, such as ion exchange, hydroxyapatite chromatograph or HPLC can also be used (see generally, Scopes, Protein Purification (Springer-Verlag, NY, 1982)).

### VII. Targets

Antibodies or fusion proteins incorporating the heavy chain modifications and multimerizing peptide described above can be made to any target molecule. The antibodies or fusion proteins are particularly useful for surface-bound target proteins (e.g., on cells or viruses) in which aggregation of the target protein induces a desired response. The desired response can be, for example, clearing of a cell or virus bearing a target, signal transduction through a receptor, e.g., inducing apoptosis or cytostasis, inhibiting a receptor binding to a ligand or counterreceptor, or internalization of an antibody or fusion protein conjugated to a toxic agent. Antibodies or fusion proteins can be made to the same targets as existing commercial antibodies or fusion proteins or can be derivatized versions of commercial antibodies or fusion proteins in which the existing constant region has been replaced by heavy chain constant regions of the present invention. The antibodies or fusion proteins can also aggregate surface-bound antigen indirectly by binding to a target ligand bound to a surface-bound antigen.

To illustrate one possible mechanism of action, an antibody or fusion protein incorporating heavy chain constant regions of the invention is generated with specificity to a member of the tumor necrosis factor (TNF) receptor superfamily. Such receptors require trimerization for signal transduction. Because the antibody or fusion protein of the invention is multivalent (e.g., dimeric, trimeric, or tetrameric), it can multimerize antigens on the surface of cells. Trimerized TNF receptor superfamily members form a complex with tumor necrosis factor receptor-associated factors (TRAFs) in the cytoplasm, which leads to induction of a wide range of cellular responses, including activation of the NF-κB and stress-activated protein kinase (SAP kinase) intracellular signal pathways, and also apoptosis, growth arrest, differentiation, and proliferation of the cells bearing the TNF receptor family member (depending on the superfamily member) (Bradley and Pober, Oncogene 20:6482-6491, 2001; Baker and Reddy, Oncogene 17:3261-3270, 1998; Chung et al., J. Cell Sci. 115:679-688, 2002; Hildebrand et al., Immunol. Rev. 244:55-74, 2011). Optionally, an antibody or fusion protein of the invention induces signal transduction in a cell bearing a TNF receptor superfamily on the surface in circumstances in which a control antibody or fusion (defined below) does not (i.e., background level indistinguishable from irrelevant control antibody). For some antibodies or fusion proteins of the invention the signal (assessed from any of the above responses) is at least 2-fold, 5-fold, 10-fold 50-fold or 100-fold greater than that of the control antibody or fusion protein. Efficacy of such multivalent antibodies or fusion protein to treat cancer or other diseases can be studied in mouse xenograft models of cancer or other appropriate animal disease models.

Some antibodies or fusion proteins of the invention which bind to a member of the TNF receptor superfamily recognize the antigen expressed on tumor cells and induce apoptosis and/or growth arrest of the tumor cells. Preferably, such antibody or fusion protein of the invention binds to CD30, TNFR1 (CD120a), FAS (CD95), DR3, DR4 (CD261), DR5 (CD262) or DR6 (CD358). More preferably, an antibody or fusion protein of the invention induces apoptosis of tumor cells bearing a TNF receptor superfamily member (e.g., Ramos cells) with an EC50 of less than 100 ng/ml or less than 10 ng/ml. The capacity of an antibody or fusion protein of the invention to induce apoptosis can be compared with a control antibody or fusion protein (i.e., an antibody having the same variable regions and IgG regions, but lacking the mutations for heterodimeric Fc-to-Fc interaction and multimer-forming polypeptides, or likewise a fusion protein having the same binding region and IgG region but lacking the mutations for heterodimeric Fc-to-Fc interaction and multimer-forming polypeptides. Under conditions in which the antibody or fusion protein of the invention induces apoptosis with an EC50 of less than 100 ng/ml, the control antibody or fusion protein sometimes induces apoptosis with an EC50 of greater than 1000 ng/ml or in some cases does not induce apoptosis (i.e., level indistinguishable from an irrelevant negative control antibody).

Other antibodies or fusion proteins of the invention bind to a member of the TNF receptor superfamily, effect trimerization of the receptor, and activate immune cells bearing the TNF receptor superfamily member (e.g., B cells, T cells, monocytes, neutrophils, NK cells, mast cells, eosinophils, basophils, macrophage, or dendritic cells) which results in one or more of the following: better survival and more proliferation of the cells, and higher production of cytokines and surface molecules by the cells (Watts, Annu. Rev. Immunol. 23:23-68, 2005; Grewal and Flavell, Annu. Rev. Immunol. 16:111-135, 1998; Hehlgans and Pfeffer, Immunology 115:1-20, 2005). More preferably, such antibody or fusion protein of the invention binds to immune costimulatory molecules of the TNF receptor superfamily (e.g., CD40, OX40, CD27, CD30, HVEM, GITR and 4-1BB), In one example, the capacity of an antibody or fusion protein of the invention to activate immune cells can be compared with a control antibody or fusion protein (i.e., an antibody having the same variable regions and IgG regions, but lacking the mutations for heterodimeric Fc-to-Fc interaction and multimer-forming polypeptides, or likewise a fusion protein having the same binding region and IgG region but lacking the mutations for heterodimeric Fc-to-Fc interaction and multimer-forming polypeptides) by measuring the expression of CD23, CD54 or CD95 on the surface (Henriquez et al., J. Immunol. 162:3298-3307, 1999). Under conditions in which the antibody or fusion protein of the invention increases CD95 expression in immune cells by 5-fold or higher, the control antibody or fusion protein sometimes increases CD95 expression by less than 2-fold. Efficacy of such multivalent antibodies to treat cancer or other diseases can be studied in mouse xenograft models of cancer or other appropriate animal models.

Other antibodies or fusion proteins of the invention bind to CD19, CD20, CD21, CD22, CD37, CD38 or CD45 expressed on the surface of normal or malignant cells, multimerize the antigens by cross-linking, and induce cell death or growth arrest of antigen-bearing cells (Ghetie et al. Proc. Natl. Acad. Sci. 94:7509-7514, 1997; Rossi et al. Cancer Res. 68:8384-8392, 2008; Lapalombella et al. Cancer Cell 21:694-708, 2012; Lund et al. Int. Immunol. 18:1029-1042, 2006; Steff et al. Crit. Rev. Immunol. 23:421-440, 2003).

To illustrate another mechanism, an antibody or fusion protein incorporating heavy chain constant regions of the invention is generated with specificity to an antigen expressed on the surface of immune cells, for example, B cells, T cells, monocytes, neutrophils or dendritic cells. Such an antibody can multimerize the antigen on the surface of immune cells and trigger normal or abnormal signal transduction. Alternatively, such an antibody can trigger internalization of the cell surface antigen. The function of such immune cells is enhanced or suppressed, depending on the antigen, type of cells and epitope bound, resulting in modulation of the immune system. The efficacy of such an antibody to treat immune disorders is studied in appropriate in vitro systems or animal models of an immune disorder.

To illustrate another mechanism, an antibody or fusion protein incorporating heavy chain constant regions of the invention is generated with specificity to an antigen expressed by a pathogen, such as infectious bacteria, yeast, fungus or virus. The antibody neutralizes the infectious microorganism or virus (e.g., by ADCC, CDC, opsonization, or by inhibiting interaction between the pathogen and a cellular receptor, or by action of a toxic moiety attached to the antibody.) The efficacy of such an antibody to treat infectious diseases can be studied in appropriate in vitro systems or animal models of infection.

Targets of interest include receptors on cancer cells and their ligands or counter-receptors (e.g., CD3, CD20, CD22, CD30, CD34, CD40, CD44, CD52 CD70, CD79a, DR4, DR5, EGFR, CA-125/Muc-16, MC1 receptor, PEM antigen, gp72, EpCAM, Her-2, VEGF or VEGFR, ganglioside GD3, CEA, AFP, CTLA-4, alpha v beta 3, HLA-DR 10 beta, SK-1). Other targets of interest are autoantibodies or T-cell subsets mediating autoimmune disease. Other targets of interest are growth factor receptors (e.g., FGFR, HGFR, PDGFR, EFGR, NGFR, and VEGFR) and their ligands. Other targets are G-protein receptors and include substance K receptor, the angiotensin receptor, the α and β adrenergic receptors, the serotonin receptors, and PAF receptor. See, e.g., Gilman, Ann. Rev. Biochem. 56:625 649 (1987). Other targets include ion channels (e.g., calcium, sodium, potassium channels), muscarinic receptors, acetylcholine receptors, GABA receptors, glutamate receptors, and dopamine receptors (see Harpold, U.S. Pat. No. 5,401,629 and U.S. Pat. No. 5,436,128). Other targets are adhesion proteins such as integrins, selectins, and immunoglobulin superfamily members (see Springer, Nature 346:425 433 (1990). Osborn, Cell 62:3 (1990); Hynes, Cell 69:11 (1992)). Other targets are cytokines, such as interleukins IL-1 through about IL-37 to-date, tumor necrosis factors, interferon, and, tumor growth factor beta, colony stimulating factor (CSF) and granulocyte monocyte colony stimulating factor (GM-CSF), and cell death receptor family members, particularly DR4 or DR5. See Human Cytokines: Handbook for Basic &amp; Clinical Research (Aggrawal et al. eds., Blackwell Scientific, Boston, Mass. 1991). Other targets are amyloidogenic peptides, such as Abeta, alpha-synuclein or prion peptide. Other targets are hormones, enzymes, and intracellular and intercellular messengers, such as, adenyl cyclase, guanyl cyclase, and phospholipase C. Target molecules can be human, mammalian or bacterial. Other targets are antigens, such as proteins, glycoproteins and carbohydrates from microbial pathogens, both viral and bacterial, and tumors. Other targets are co-stimulatory molecules, such as CD40, OX40, 4-1BB, GITR and CD27. Agonizing such molecules stimulates the immune system and is useful for immunotherapy against cancer or infectious agents.

Some examples of commercial antibodies and their targets include alemtuzumab, CD52, rituximab, CD20, trastuzumab Her/neu, nimotuzumab, cetuximab, EGFR, bevacizumab, VEGF, palivizumab, RSV, abciximab, GpIIb/IIIa, infliximab, adalimumab, certolizumab, golimumab TNF-alpha, baciliximab, daclizumab, IL-2, omalizumab, IgE, gemtuzumab, CD33, natalizumab, VLA-4, vedolizumab alpha4beta7, belimumab, BAFF, otelixizumab, teplizumab CD3, ofatumumab, ocrelizumab CD20, epratuzumab CD22, alemtuzumumab CD52, eculizumab C5, canakimumab IL-1beta, mepolizumab IL-5, reslizumab, tocilizumab IL-6R, ustekinumab, briakinumab IL-12, 23. Examples of commercial fusion proteins include etanercept which binds TNF-alpha, alefacept (LFA3-Fc fusion which binds CD2), TACI-Fc fusion which binds BAFF and APRIL, abatacept (CTLA-4-Fc which binds CD80 and CD86), and romiplostim (a peptide analog of thrombopoietin fused to Fc). Any of the commercial antibodies or fusion protein can be modified to replace the existing heavy chain constant region with heavy chain constant regions of the invention. Alternatively, heavy chain constant regions of the invention region can be linked to other antibodies with the same target specificity (e.g., as determined by a competition assay) as any of the above commercial antibodies or fusion proteins.

### VIII. Immunoconjugates

Antibodies or fusion proteins can be conjugated to a toxic agent. Toxic agents can be cytotoxic or cystostatic. Some example of toxic agents include antitubulin agents, auristatins, DNA minor groove binders, DNA replication inhibitors, alkylating agents (e.g., platinum complexes such as cis-platin, mono(platinum), bis(platinum) and tri-nuclear platinum complexes and carboplatin), anthracyclines, antibiotics, antifolates, antimetabolites, chemotherapy sensitizers, duocarmycins, camptothecins, etoposides, fluorinated pyrimidines, ionophores, lexitropsins, nitrosoureas, platinols, pre-forming compounds, purine antimetabolites, puromycins, radiation sensitizers, steroids, taxanes, topoisomerase inhibitors, vinca alkaloids, or the like. A variety of radionuclides are available for the production of radioconjugated antibodies. Examples include ²¹²Bi, ¹³¹I, ¹³¹In, ⁹⁰Y, and ¹⁸⁶Re. Conjugates of an antibody and toxic agent can be made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis(p-azidobenzoyl)hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). A toxic agent can also be linked to an antibody via a linker, which may be cleavable under intracellular conditions (US 2003-0083263, 2005-0238649 and 2005-0009751). Many of the above toxic agents are only effective or most effective when internalized within a cell. The antibodies or fusion proteins of the invention can be internalized by binding to cellular receptors, for example, crosslinking of cellular receptors can promote internalization.

### IX. Methods of Treatment and Pharmaceutical Compositions

The antibodies or fusion proteins of the invention can be used for treating cancers including those for which commercial antibodies mentioned above have been used. The methods can be used to treat solid tumors, and particularly hematological malignancies, such as leukemia (e.g., T cell large granular lymphocyte leukemia), lymphoma (Hodgkin's or Non-Hodgkin's), or multiple myeloma. Solid tumors include skin (e.g., melanoma), ovarian, endometrial, bladder, breast, rectum, colon, gastric, pancreatic, lung, thymus, kidney and brain.

The antibodies and fusion protein of the invention can also be used for suppressing various undesirable immune responses including those in which the commercial antibodies mentioned above have been used.

One category of immune disorders treatable by antibodies or fusion proteins of the invention is transplant rejection. When allogeneic cells or organs (e.g., skin, kidney, liver, heart, lung, pancreas and bone marrow) are transplanted into a host (i.e., the donor and donee are different individual from the same species), the host immune system is likely to mount an immune response to foreign antigens in the transplant (host-versus-graft disease) leading to destruction of the transplanted tissue. The antibodies of the present invention are useful, inter alia, to block alloantigen-induced immune responses in the donee.

A related use for antibodies or fusion proteins of the present invention is in modulating the immune response involved in “graft versus host” disease (GVHD). GVHD is a potentially fatal disease that occurs when immunologically competent cells are transferred to an allogeneic recipient. In this situation, the donor's immunocompetent cells may attack tissues in the recipient. Tissues of the skin, gut epithelia and liver are frequent targets and may be destroyed during the course of GVHD. The disease presents an especially severe problem when immune tissue is being transplanted, such as in bone marrow transplantation; but less severe GVHD has also been reported in other cases as well, including heart and liver transplants.

A further situation in which immune suppression is desirable is in treatment of autoimmune diseases such as type 1 diabetes, Crohn's disease, ulcerative colitis, multiple sclerosis, stiff man syndrome, rheumatoid arthritis, myasthenia gravis and lupus erythematosus. In these diseases, the body develops a cellular and/or humoral immune response against one of its own antigens leading to destruction of that antigen, and potentially crippling and/or fatal consequences. Autoimmune diseases are treated by administering one of the antibodies or fusion proteins of the invention.

Other immune disorders treatable by antibodies or fusion proteins of the invention, include asthma, allergies, celiac disease, psoriasis, and uveitis. Celiac disease, psoriasis and uveitis are autoimmune diseases.

The antibodies or fusion protein can also be used for treatment of pathogenic infections, such as viral, bacterial, protozoan or fungal infection. Some example of viral infections include HIV, hepatitis (A, B, or C), herpes virus (e.g., VZV, HSV-1, HAV-6, HSV-II, CMV, and Epstein Barr virus), adenovirus, XMRV, influenza virus, flaviviruses, echovirus, rhinovirus, coxsackie virus, cornovirus, respiratory syncytial virus, mumps virus, rotavirus, measles virus, rubella virus, parvovirus, vaccinia virus, HTLV virus, dengue virus, MLV-related Virus, papillomavirus, molluscum virus, poliovirus, rabies virus, JC virus and arboviral encephalitis virus. Some examples of bacterial infections include chlamydia, rickettsial bacteria, mycobacteria, staphylococci, streptococci, pneumonococci, meningococci and conococci, klebsiella, proteus, serratia, pseudomonas, legionella, diphtheria, salmonella, bacilli, cholera, tetanus, botulism, anthrax, plague, leptospirosis, Lymes disease bacteria, streptococci, or neisseria. Some examples of pathogenic fungi include Candida, Aspergillus, Cryptococcus, Histoplasma, Pneumocystis and Stachybotrys. Examples of protozoa include Cryptosporidium, Giardia lamblia and plasmodium.

Antibodies or fusion proteins are administered in an effective regime meaning a dosage, route of administration and frequency of administration that delays the onset, reduces the severity, inhibits further deterioration, and/or ameliorates at least one sign or symptom of a disorder. If a patient is already suffering from a disorder, the regime can be referred to as a therapeutically effective regime. If the patient is at elevated risk of the disorder relative to the general population but is not yet experiencing symptoms, the regime can be referred to as a prophylactically effective regime. In some instances, therapeutic or prophylactic efficacy can be observed in an individual patient relative to historical controls or past experience in the same patient. In other instances, therapeutic or prophylactic efficacy can be demonstrated in a preclinical or clinical trial in a population of treated patients relative to a control population of untreated patients.

Exemplary dosages for an antibody or fusion protein are 0.01-20, or 0.5-5, or 0.01-1, or 0.01-0.5 or 0.05-0.5 mg/kg body weight (e.g., 0.1, 0.5, 1, 2, 3, 4 or 5 mg/kg) or 10-1500 mg as a fixed dosage. The dosage depends on the condition of the patient and response to prior treatment, if any, whether the treatment is prophylactic or therapeutic and whether the disorder is acute or chronic, among other factors.

Administration can be parenteral, intravenous, oral, subcutaneous, intra-arterial, intracranial, intrathecal, intraperitoneal, topical, intranasal or intramuscular. Administration into the systemic circulation by intravenous or subcutaneous administration is preferred. Intravenous administration can be, for example, by infusion over a period such as 30-90 min.

The frequency of administration depends on the half-life of the antibody or fusion protein in the circulation, the condition of the patient and the route of administration among other factors. The frequency can be daily, weekly, monthly, quarterly, or at irregular intervals in response to changes in the patient's condition or progression of the disorder being treated. An exemplary frequency for intravenous administration is between weekly and quarterly over a continuous cause of treatment, although more or less frequent dosing is also possible. For subcutaneous administration, an exemplary dosing frequency is daily to monthly, although more or less frequent dosing is also possible.

The number of dosages administered depends on whether the disorder is acute or chronic and the response of the disorder to the treatment. For acute disorders or acute exacerbations of chronic disorders between 1 and 10 doses are often sufficient. Sometimes a single bolus dose, optionally in divided form, is sufficient for an acute disorder or acute exacerbation of a chronic disorder. Treatment can be repeated for recurrence of an acute disorder or acute exacerbation. For chronic disorders, an antibody can be administered at regular intervals, e.g., weekly, fortnightly, monthly, quarterly, every six months for at least 1, 5 or 10 years, or the life of the patient.

Pharmaceutical compositions for parenteral administration are preferably sterile and substantially isotonic and manufactured under GMP conditions. Pharmaceutical compositions can be provided in unit dosage form (i.e., the dosage for a single administration). Pharmaceutical compositions can be formulated using one or more physiologically acceptable carriers, diluents, excipients or auxiliaries. The formulation depends on the route of administration chosen. For injection, antibodies can be formulated in aqueous solutions, preferably in physiologically compatible buffers such as Hank's solution, Ringer's solution, or physiological saline or acetate buffer (to reduce discomfort at the site of injection). The solution can contain formulatory agents such as suspending, stabilizing and/or dispersing agents. Alternatively antibodies can be in lyophilized form for constitution with a suitable vehicle, e.g., sterile pyrogen-free water, before use.

Treatment with antibodies of the invention can be combined with other treatments effective against the disorder being treated. For treatment of immune disorders, conventional treatments include mast cell degranulation inhibitors, corticosteroids, nonsteroidal anti-inflammatory drugs, and stronger anti-inflammatory drugs such as azathioprine, cyclophosphamide, leukeran, FK506 and cyclosporine. Biologic anti-inflammatory agents, such as Tysabri® (natalizumab) or Humira® (adalimumab), can also be used. When used in treating cancer, the antibodies of the invention can be combined with chemotherapy, radiation, stem cell treatment, surgery or treatment with other biologics such as Herceptin® (trastuzumab) against the HER2 antigen, Avastin® (bevacizumab) against VEGF, or antibodies to the EGF receptor, such as (Erbitux®, cetuximab), and Vectibix® (panitumumab). Chemotherapy agents include chlorambucil, cyclophosphamide or melphalan, carboplatinum, daunorubicin, doxorubicin, idarubicin, and mitoxantrone, methotrexate, fludarabine, and cytarabine, etoposide or topotecan, vincristine and vinblastine. For infections, treatment can be in combination with antibiotics, anti-virals, anti-fungal or anti-protozoan agents or the like.

### X. Other Applications

The antibodies or fusion proteins can be used for detecting their target molecule in the context of clinical diagnosis or treatment or in research. For example, the antibodies can be used to detect a cancer-related antigen as an indication a patient is suffering from an immune mediated disorder amenable to treatment. The antibodies can also be sold as research reagents for laboratory research in detecting targets and their response to various stimuli. In such uses, antibodies or fusion proteins can be labeled with fluorescent molecules, spin-labeled molecules, enzymes or radioisotypes, and can be provided in the form of kit with all the necessary reagents to perform the assay. The antibodies or fusion protein can also be used to purify their target antigens e.g., by affinity chromatography.

All patent filings, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise if different versions of a publication, website or the like are published at different times, the version most recently published at the effective filing date of the application is meant unless otherwise indicated. Any feature, step, element, embodiment, or aspect of the invention can be used in combination with any other unless specifically indicated otherwise. Although the present invention has been described in some detail by way of illustration and example for purposes of clarity and understanding, it will be apparent that certain changes and modifications may be practiced within the scope of the appended claims.

### EXAMPLES

### Example 1

### Expression Vectors for Trimeric IgG Antibodies

Gene cloning, mutagenesis and plasmid construction in this work was carried out with standard molecular biology techniques such as those described in Sambrook and Russel (Molecular Cloning, A Laboratory Manual, 3rd ed., 2001, Cold Spring Harbor Laboratory Press, Cold Spring Harbor, N.Y.), Kostelny et al. (Int. J. Cancer 93:556-565, 2001), Cole et al. (J. Immunol. 159:3613-3621, 1997) and Tsurushita et al. (Methods 36:69-83, 2005).

The mouse hybridoma producing anti-human death receptor 4 (DR4; also called Apo2, TRAIL receptor 1 and TNFRSF10A) monoclonal IgG1/lambda antibody YON007 was generated at JN Biosciences (Mountain View, Calif.) using the extracellular region of human DR4 fused to the Fc region of human gamma-1 heavy chain (DR4-Fc) (SEQ ID NO:1) as immunogens and following standard hybridoma techniques such as the GenomONE CF EX cell fusion reagent (Cosmo Bio, Carlsbad, Calif.) (U.S. 61/679,045). Humanization of the YON007 VH and VL regions to generate HuYON007 VH and VL, respectively, was carried out by the procedure described by Tsurushita et al. (supra).

A gene encoding HuYON007 VH was synthesized as an exon including a splice donor signal at the 3′ end of the coding region, a SpeI site at the 5′ end of the fragment, and a HindIII site at the 3′ end of the fragment. The amino acid sequence of HuYON007 VH, including the signal peptide, is MNRLTSSLLLLIVPAYVLSQVTLRESGPALVKPTQTLTLTCTFSGFSLSTSGMGVSWIRQPPGKALEWLAHIYW DDDKRYNPSLKSRLTISKDTSKNQVVLTMTNMDPVDTATYYCTRRGEYGNFDYWGQGTLVTVSS (SEQ ID NO:2). The mature HuYON007 VH sequence starts at position 20 in SEQ ID NO:2.

A gene encoding HuYON007 VL was synthesized as an exon including a splice donor signal at the 3′ end of the coding region, a NheI site at the 5′ end of the fragment, and an EcoRI site at the 3′ end of the fragment. The amino acid sequence of HuYON007 VL is MAWISLILSLLALSSGAISQTVVTQEPSFSVSPGGTVTLTCRSSSGAVTTSNFANWVQQTPGQAPRGLIGGTN NRAPGVPDRFSGSLLGNKAALTITGAQADDESDYYCALWYSNHWVFGGGTKLTVL (SEQ ID NO:3). The mature HuYON007 VL sequence starts at position 20 in SEQ ID NO:3.

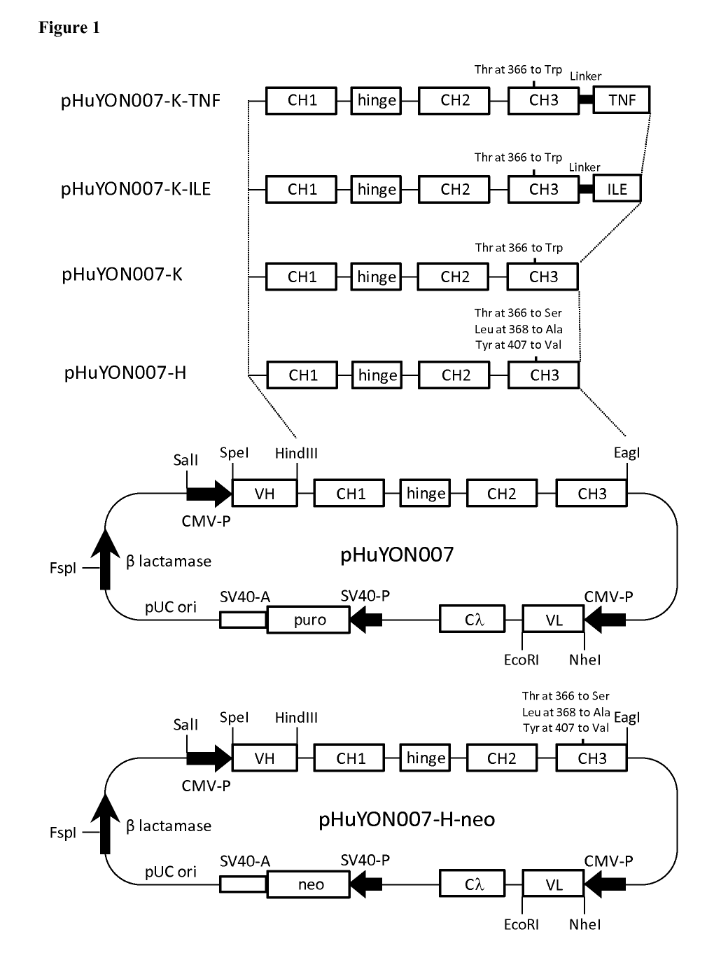

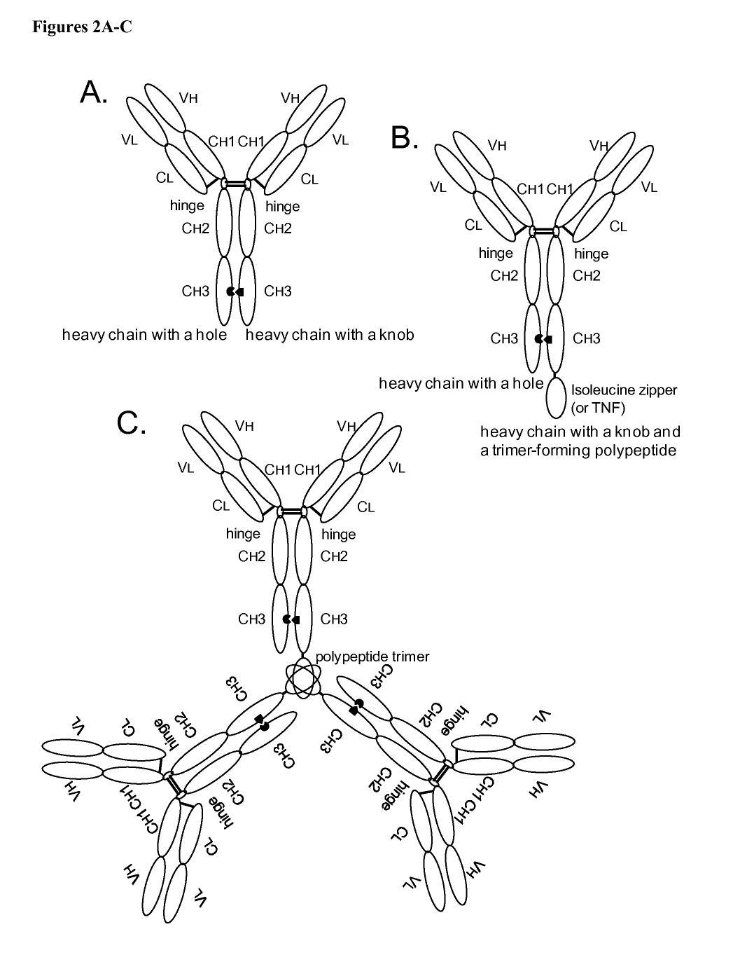

The mammalian expression vector pHuYON007 (FIG. 1) for production of a humanized anti-human DR4 IgG1/lambda antibody (HuYON007) contains the following genetic components. Proceeding clockwise from the SalI site of pHuYON007 in FIG. 1, the plasmid contains the heavy chain transcription unit starting with the human cytomegalovirus (CMV) major immediate early promoter and enhancer (CMV-P in the figure) to initiate transcription of the antibody heavy chain gene. The CMV promoter is followed by an exon encoding the heavy chain variable region of the humanized anti-human DR4 monoclonal antibody HuYON007 flanked by the SpeI and HindIII sites (VH), a genomic sequence containing the human gamma-1 heavy chain constant regions including the CH1, hinge, CH2 and CH3 exons with the intervening introns, and the polyadenylation site of the human gamma-1 heavy chain gene. After the heavy chain gene sequence, the light chain transcription unit begins with the CMV promoter and enhancer (CMV-P), followed by an exon encoding the light chain variable region of the humanized anti-human DR4 monoclonal antibody HuYON007 flanked by the NheI and EcoRI sites (VL), a genomic sequence containing the human lambda chain constant region exon (CX) with an intron preceding it, and the polyadenylation site of the human lambda chain gene following the CX exon. The light chain gene is then followed by the SV40 early promoter (SV40-P), the puromycin N-acetyl-transferase gene (puro) for resistance to puromycin, and a segment containing the SV40 polyadenylation site (SV40-A). Finally, pHuYON007 contains a part of the plasmid pUC19, comprising the bacterial origin of replication (pUC on) and the β lactamase gene (β lactamase). Arrows in the figure indicate the orientation of transcription. The amino acid sequence of the heavy chain constant region, which comprises the CH1, hinge, CH2 and CH3 regions, in pHuYON007 is ASTKGPSVFPLAPSSKSTSGGTAALGCLVKDYFPEPVTVSWNSGALTSGVHTFPAVLQSSGLYSLSSVVTVPSS SLGTQTYICNVNHKPSNTKVDKKVEPKSCDKTHTCPPCPAPELLGGPSVFLFPPKPKDTLMISRTPEVTCVVVD VSHEDPEVKFNWYVDGVEVHNAKTKPREEQYNSTYRVVSVLTVLHQDWLNGKEYKCKVSNKALPAPIEKTIS KAKGQPREPQVYTLPPSRDELTKNQVSLTCLVKGFYPSDIAVEWESNGQPENNYKTTPPVLDSDGSFFLYSKL TVDKSRWQQGNVFSCSVMHEALHNHYTQKSLSLSPGK (SEQ ID NO:4).

For expression of trimeric IgG antibodies, the heavy chain gene in pHuYON007 was first modified in two different ways. In one of the two resulting expression vectors (pHuYON007-H; FIG. 1), three amino acid substitutions (Thr to Ser at position 366, Leu to Ala at position 368, and Tyr to Val at position 407; T366S, L368A and Y407V, respectively) were introduced in the CH3 region by site-directed mutagenesis. Eu numbering by Kabat et al. (Sequences of Proteins of Immunological Interest, National Institutes of Health, Bethesda, Md., 1987 and 1991) is used for assigning positions of amino acids in the human gamma heavy chain. The modified gamma heavy chain expressed from pHuYON007-H serves as a hole of the knobs-into-holes structure for hetero-dimeric Fc-to-Fc interaction (Atwell et al., J. Mol. Biol. 270:26-35, 1997). The amino acid sequence of the modified heavy chain constant region encoded in pHuYON007-His ASTKGPSVFPLAPSSKSTSGGTAALGCLVKDYFPEPVTVSWNSGALTSGVHTFPAVLQSSGLYSLSSVVTVPSS SLGTQTYICNVNHKPSNTKVDKKVEPKSCDKTHTCPPCPAPELLGGPSVFLFPPKPKDTLMISRTPEVTCVVVD VSHEDPEVKFNWYVDGVEVHNAKTKPREEQYNSTYRVVSVLTVLHQDWLNGKEYKCKVSNKALPAPIEKTIS KAKGQPREPQVYTLPPSRDELTKNQVSLSCAVKGFYPSDIAVEWESNGQPENNYKTTPPVLDSDGSFFLVSKL TVDKSRWQQGNVFSCSVMHEALHNHYTQKSLSLSPGK (SEQ ID NO:5). The expression vector pHuYON007-H was further modified by replacing the puromycin N-acetyl-transferase gene (puro) with the neomycin resistance gene (neo) for selection with G418. The resultant plasmid was named pHuYON007-H-neo (FIG. 1)

In the other construct (pHuYON007-K; FIG. 1), an amino acid substitution (Thr to Trp at position 366; T366W) was introduced in the CH3 region. The heavy chain constant region carrying the T366W mutation serves as a knob of the knobs-into-holes structure for hetero-dimeric Fc-to-Fc interaction (Atwell et al., supra). The amino acid sequence of the modified heavy chain constant region encoded in pHuYON007-K is ASTKGPSVFPLAPSSKSTSGGTAALGCLVKDYFPEPVTVSWNSGALTSGVHTFPAVLQSSGLYSLSSVVTVPSS SLGTQTYICNVNHKPSNTKVDKKVEPKSCDKTHTCPPCPAPELLGGPSVFLFPPKPKDTLMISRTPEVTCVVVD VSHEDPEVKFNWYVDGVEVHNAKTKPREEQYNSTYRVVSVLTVLHQDWLNGKEYKCKVSNKALPAPIEKTIS KAKGQPREPQVYTLPPSRDELTKNQVSLWCLVKGFYPSDIAVEWESNGQPENNYKTTPPVLDSDGSFFLYSK LTVDKSRWQQGNVFSCSVMHEALHNHYTQKSLSLSPGK (SEQ ID NO:6).

The heavy chain gene encoded in pHuYON007-K was further modified by fusing a polypeptide linker followed by a polypeptide known as an isoleucine zipper capable of forming homo-trimers (Harbury et al. Nature 371:80-83, 1994) at the carboxyl terminus of the CH3 region. The resultant expression vector was named pHuYON007-K-ILE (FIG. 1). The amino acid sequence of the modified heavy chain constant region encoded in pHuYON007-K-ILE is ASTKGPSVFPLAPSSKSTSGGTAALGCLVKDYFPEPVTVSWNSGALTSGVHTFPAVLQSSGLYSLSSVVTVPSS SLGTQTYICNVNHKPSNTKVDKKVEPKSCDKTHTCPPCPAPELLGGPSVFLFPPKPKDTLMISRTPEVTCVVVD VSHEDPEVKFNWYVDGVEVHNAKTKPREEQYNSTYRVVSVLTVLHQDWLNGKEYKCKVSNKALPAPIEKTIS KAKGQPREPQVYTLPPSRDELTKNQVSLWCLVKGFYPSDIAVEWESNGQPENNYKTTPPVLDSDGSFFLYSK LTVDKSRWQQGNVFSCSVMHEALHNHYTQKSLSLSPGKSGGGSGGGSMKQIEDKIEEILSKIYHIENEIARIKK LIGERAG (SEQ ID NO:7).

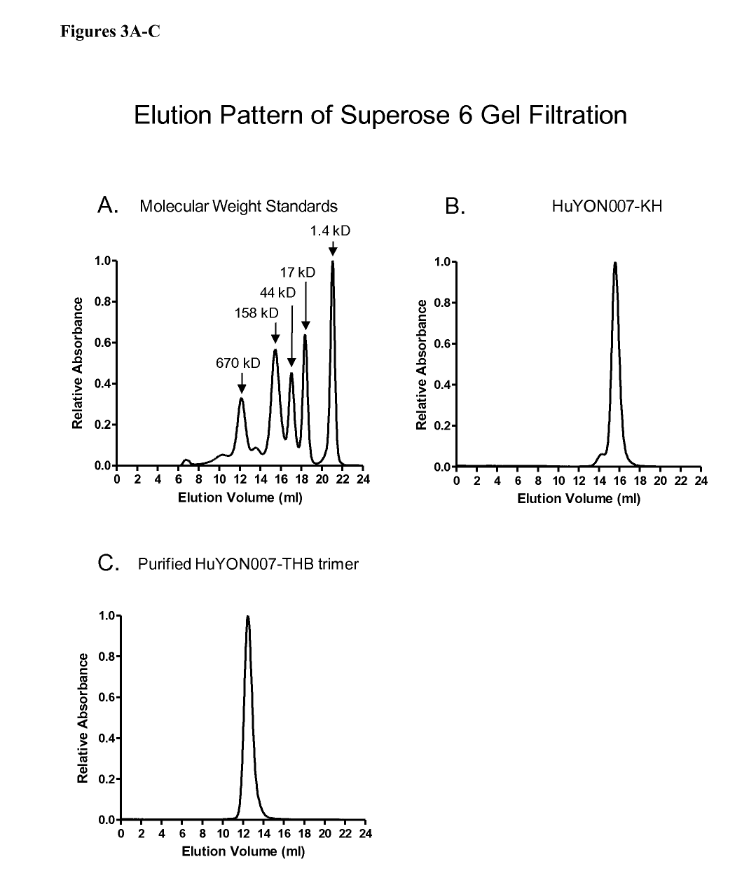

Heavy chains encoded in pHuYON007-H and pHuYON007-H-neo are identical to each other in their amino acid sequence. Light chains encoded in pHuYON007-H, pHuYON007-H-neo and pHuYON007-K are identical to each other in their amino acid sequence. Heavy chains expressed from pHuYON007-H (or pHuYON007-H-neo) and heavy chains expressed from pHuYON007-K preferentially form Fc-to-Fc heterodimeric molecules (Atwell, supra). When heavy and light chains from pHuYON007-H (or pHuYON007-H-neo) and pHuYON007-K are expressed simultaneously in cells, monomeric HuYON007 antibodies each composed of one HuYON007 heavy chain from pHuYON007-H (or pHuYON007-H-neo), one HuYON007 heavy chain from pHuYON007-K, and two HuYON007 light chains (HuYON007-KH; schematically illustrated in FIG. 2A) are produced.

Light chains encoded in pHuYON007-H, pHuYON007-H-neo and pHuYON007-K-ILE are identical to each other in their amino acid sequence. Heavy chains expressed from pHuYON007-H (or pHuYON007-H-neo) and heavy chains expressed from pHuYON007-K-ILE preferentially form Fc-to-Fc heterodimeric molecules (Atwell, supra). When heavy and light chains from pHuYON007-H (or pHuYON007-H-neo) and pHuYON007-K-ILE are expressed simultaneously in cells, HuYON007 antibodies each composed of one HuYON007 heavy chain from pHuYON007-H, one HuYON007 heavy chain from pHuYON007-K-ILE, and two HuYON007 light chains (HuYON007-THB; schematically illustrated as a monomer in FIG. 2B) are produced. Furthermore, HuYON007-THB antibodies form trimers due to homo-trimeric association of the isoleucine zipper fused to the carboxyl terminus of the CH3 region of the heavy chain produced from pHuYON007-K-ILE (Harbury et al., supra). The structure of trimeric HuYON007-THB is schematically illustrated in FIG. 2C.

### Example 2

### Expression of Trimeric Anti-DR4 IgG Antibodies

The expression vectors pHuYON007-H and pHuYON007-K-ILE were individually or together transfected into the human embryonic kidney cell line HEK293 using Lipofectamine 2000 reagent (Invitrogen, Carlsbad, Calif.) following the manufacture's protocol. HEK293 cells were grown in DME media containing 10% fetal bovine serum (FBS; HyClone, Logan, Utah) at 37° C. in a 7.5% CO₂ incubator. Culture supernatants containing transiently expressed HuYON007 antibodies were fractionated by gel filtration using the AKTA Basic FPLC system with a Superose 6 10/300 GL column which has a separation range from 5 to 5,000 kilo Dalton (kDa) of globular proteins (GE Healthcare, Indianapolis, Ind.). PBS (phosphate-buffered saline, pH 7.4) was used as elution buffer.

Presence of HuYON007 antibodies in each Superose 6 fraction was analyzed by sandwich ELISA. In a typical experiment, an ELISA plate was coated with goat anti-human gamma heavy chain polyclonal antibody in PBS, washed with Wash Buffer (PBS containing 0.05% Tween 20), and blocked with Blocking Buffer (PBS containing 2% Skim Milk and 0.05% Tween 20). After washing with Wash Buffer, test samples appropriately diluted in ELISA Buffer (PBS containing 1% Skim Milk and 0.025% Tween 20) were applied to the ELISA plate. After incubating the ELISA plate for 1 hr at room temperature and washing with Wash Buffer, bound HuYON007 antibodies were detected using HRP-conjugated goat anti-human lambda chain polyclonal antibody. After incubation and washing, color development was initiated by adding ABTS substrate and stopped with 2% oxalic acid. Absorbance was read at 405 nm.

When pHuYON007-H alone was transfected into HEK293 cells, a single major peak of the ELISA signal for the presence of IgG1/lambda antibodies was observed in the Superose 6 fraction corresponding to roughly 150 kDa proteins, which is the expected size of monomeric HuYON007 IgG antibodies produced from pHuYON007-H. This is consistent with the observation by Atwell et al. (supra) that the Fc region having the hole mutation can associate with each other to form Fc-to-Fc homo-dimeric molecules.

When pHuYON007-K-ILE alone was transfected into HEK293 cells, the peak of the ELISA signal was observed in the Superose 6 fraction corresponding to roughly 250 kDa proteins. The major species of HuYON007 antibodies produced from pHuYON007-K-ILE in HEK293 cells is likely to be composed of three light chains (approximately 25 kDa each) and three heavy chains (approximately 55 kDa each) associated with each other to form trimers due to the presence of the isoleucine zipper at the carboxyl terminus of each heavy chain.

When pHuYON007-H and pHuYON007-K-ILE were cotransfected, the major peak of the ELISA signal for the presence of IgG1/lambda antibodies was observed in the Superose 6 fractions corresponding to roughly 500 kDa proteins, and thus indicating formation of trimeric HuYON007 antibodies (FIG. 2C) each composed of three heavy chains from pHuYON007-H, three heavy chains from pHuYON007-K-ILE, and six HuYON007 light chains.

### Example 3

### Purification and Characterization of Trimeric Anti-DR4 IgG Antibodies

The expression vectors pHuYON007-H-neo and pHuYON007-K-ILE were introduced together into the chromosomes of a Chinese hamster ovary cell line CHO-K1 (ATCC, Manassas, Va.) to obtain cell lines stably producing HuYON007-THB. Separately, the expression vectors pHuYON007-H and pHuYON007-K were cotransfected into CHO-K1 cells to obtain cell lines producing HuYON007-KH.

CHO-K1 cells were grown in SFM4CHO media (HyClone) at 37° C. in a 7.5% CO₂ incubator. Stable transfection into CHO-K1 was carried out by electroporation. Before transfection, each expression vector was linearized using Fspl. In a typical experiment, approximately 10⁷ cells were transfected with 20 μg of linearized plasmid, suspended in SFM4CHO media, and plated into several 96-well plates after appropriate dilutions of cells. After 48 hr, appropriate selection media was added for isolation of stable transfectants. Approximately ten days after the initiation of selection, culture supernatants of transfectants were assayed for antibody production.

Expression of HuYON007 antibodies was measured by sandwich ELISA as described above. An appropriate human or humanized IgG/lambda antibody was used as a standard. CHO-K1 stable transfectants producing each of HuYON007-THB and HuYON007-KH were expanded in SFM4-CHO until the cell viability became less than 50%. After centrifugation and filtration, culture supernatants were stored at 4° C. For antibody purification, culture supernatants were loaded onto a Protein A column (HiTrap MABSelect SuRe, GE Healthcare, Piscataway, N.J.). The column was washed with PBS before the antibody was eluted with 0.1 M glycine-HCl (pH 3.0). Buffer of eluted antibodies was neutralized with 1 M Tris-HCl (pH 8) and then changed to PBS by dialysis. Antibody concentration was determined by measuring absorbance at 280 nm (1 mg/ml=1.4 OD).

The molecular size of purified HuYON007-KH and HuYON007-THB in the native form was analyzed by gel filtration using a Superose 6 column as described above. A single dominant peak was observed for purified HuYON007-KH. When compared to the elution pattern of molecular size markers, the size of HuYON007-KH in the native form was estimated to be approximately 150 kDa, which is consistent with the size of a monomeric human IgG1 antibody composed of two heavy and two light chains. Purified HuYON007-THB showed two peaks in the elution pattern; a minor peak corresponding to approximately 160 kDa, which is the size of monomeric HuYON007-THB antibodies (FIG. 2B) and a major peak corresponding to roughly 600 kDa, which is consistent with the size of trimeric HuYON007-THB antibodies (FIG. 2C). Trimeric HuYON007-THB antibodies were fractionated by gel filtration using a Superose 6 column for further analyses. The Superose 6 elution pattern of purified HuYON007-HK and trimeric HuYON007-THB antibodies is shown in FIGS. 3B and 3C, respectively compared with molecular weight standards (FIG. 3A).

SDS-PAGE analysis under denaturing conditions indicated that trimeric HuYON007-THB was composed of three polypeptides. The largest polypeptide of approximately 55 kDa corresponds to heavy chains expressed from pHuYON007-K-ILE. The second largest polypeptide of approximately 50 kDa corresponds to heavy chains expressed from pHuYON007-H. The intensity of 55 kDa and 50 kDa bands was very similar to each other. The smallest polypeptide of approximately 25 kDa corresponds to light chains expressed from both pHuYON007-K-ILE and pHuYON007-H. HuYON007-KH appeared to be composed of two polypeptides in SDS-PAGE analysis under denaturing conditions. The larger polypeptide of approximately 50 kDa corresponds to two distinct, but nearly identical, heavy chains expressed from pHuYON007-K and pHuYON007-H. The smaller polypeptide of approximately 25 kDa corresponds to HuYON007 light chains.

### Example 4

### Induction of Apoptosis by Trimeric Anti-DR4 Antibodies

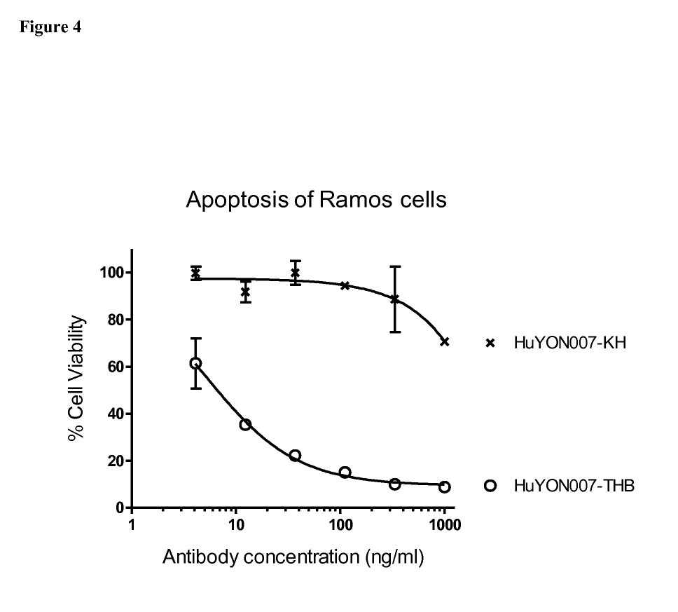

The human Burkett's lymphoma cell line Ramos expresses DR4 on the cell surface (Daniel et al. Blood:110:4037-4046, 2007). Multimerization of DR4 on the surface by cross-linking is known to induce apoptosis of cells (Griffith et al. J. Immunol. 162:2597-2605, 1999). Ramos cells (CRL-1596; ATCC, Manassas, Va.) were grown in DME media containing 10% FBS at 37° C. in a 7.5% CO₂ incubator. To assess the ability of purified HuYON007-KH (FIG. 2A) and trimeric HuYON007-THB (FIG. 2C) to induce apoptosis of Ramos cells via cross-linking of DR4 on the surface, each of these two purified HuYON007 antibodies was incubated with Ramos cells in duplicate wells at various concentrations. After overnight incubation, cell viability was measured with alamar Blue (Invitrogen) according to the manufacturer's protocol. Percent cell viability was calculated by normalizing the absorbance value in the presence of test antibodies to that in the absence of test antibodies. The absorbance value with no cells was used as background. Trimeric HuYON007-THB induced apoptosis of Ramos cells more efficiently than HuYON007-KH did (FIG. 4). The EC₅₀ value to induce apoptosis was 6 ng/ml for trimeric HuYON007-THB and more than 1,000 ng/ml for HuYON007-KH, thus showing efficient induction of apoptosis by trimeric anti-DR4 antibodies.

### Example 5

### Expression, Purification and Characterization of a Different Form of Trimeric Hexavalent Anti-DR4 Antibodies

Expression of a different form of trimeric IgG antibodies can be achieved by replacing the coding region of isoleucine zipper in pHuYON007-K-ILE with a coding region of another trimerizing peptide, include a trimer-forming domain derived from TNF superfamily members (Bodmer et al., Trends Biochem. Sci. 27:19-26, 2002; Croft et al., Nat. Rev. Drug Discovery 12:147-168, 2013), C-type lectins (Zelensky, FEBS J. 272:6179-6217, 2055) including collectins (Hakansson et al., Protein. Sci. 9:1607-1617, 2000) and tetranectin (Nielsen et al., FEBS Lett. 412:388-396, 1997), and collagens (Hulmes, J. Struc. Biol. 137:2-10, 2002), and then expressing such modified heavy chain gene together with the heavy and light chain genes in pHuYON007-H.

To obtain another example of trimeric IgG antibodies of this invention, the isoleucine zipper-coding region in pHuYON007-K-ILE was replaced by a DNA fragment encoding soluble human tumor necrosis factor (TNF; also called TNFSF1A). In addition, an amino acid at position 87 of TNF was changed from Tyr to Ser (Y87S) to eliminate its interaction with TNF receptors without losing its ability to form a trimer (Zhang et al., J. Mol. Biol. 267:24069-24075, 1992). The resultant expression vector was named pHuYON007-K-TNF (FIG. 1). The amino acid sequence of the modified heavy chain constant region in pHuYON007-K-TNF is ASTKGPSVFPLAPSSKSTSGGTAALGCLVKDYFPEPVTVSWNSGALTSGVHTFPAVLQSSGLYSLSSVVTVPSS SLGTQTYICNVNHKPSNTKVDKKVEPKSCDKTHTCPPCPAPELLGGPSVFLFPPKPKDTLMISRTPEVTCVVVD VSHEDPEVKFNWYVDGVEVHNAKTKPREEQYNSTYRVVSVLTVLHQDWLNGKEYKCKVSNKALPAPIEKTIS KAKGQPREPQVYTLPPSRDELTKNQVSLWCLVKGFYPSDIAVEWESNGQPENNYKTTPPVLDSDGSFFLYSK LTVDKSRWQQGNVFSCSVMHEALHNHYTQKSLSLSPGKSGGGGSVRSSSRTPSDKPVAHVVANPQAEGQL QWLNRRANALLANGVELRDNQLVVPSEGLYLIYSQVLFKGQGCPSTHVLLTHTISRIAVSSQTKVNLLSAIKSP CQRETPEGAEAKPWYEPIYLGGVFQLEKGDRLSAEINRPDYLDFAESGQVYFGIIAL (SEQ ID NO:8).

Simultaneous expression of heavy and light chains from pHuYON007-H and pHuYON007-K-TNF is expected to produce HuYON007 antibodies (HuYON007-THA), each of which is composed of one heavy chain from pHuYON007-H, one heavy chain from pHuYON007-K-TNF and two HuYON007 light chains as a monomer (FIG. 2B). HuYON007-THA forms trimers (FIG. 2C) through homo-trimeric association of TNF fused to heavy chains expressed from pHuYON007-K-TNF.

CHO-K1 cells stably producing HuYON007-THA were obtained by cotransfection of pHuYON007-H and pHuYON007-K-TNF as described above. HuYON007-THA antibodies were purified using protein A column chromatography as described above. Gel filtration analysis using a Superose 6 column as described above showed two major peaks in the elution pattern; one peak at approximately 180 kDa which corresponds to monomeric HuYON007-THA antibodies (FIG. 2B) and another peak at roughly 640 kDa which corresponds to trimeric HuYON007-THA antibodies (FIG. 2C).

Protein A-purified HuYON007-THA antibodies corresponding to the trimer size were fractionated by gel filtration using a Superose 6 column. SDS-PAGE analysis of such fractionated HuYON007-THA antibodies under denaturing conditions indicated that trimeric HuYON007-THA was composed of three polypeptides. Their sizes were approximately 65 kDa, 50 kDa, and 25 kDa, which correspond to the size of heavy chains expressed from pHuYON007-K-TNF, heavy chains expressed from pHuYON007-H, and HuYON007 light chains, respectively.

The ability of trimeric HuYON007-THA to induce apoptosis of Ramos cells was analyzed as described in Example 4. The viability of Ramos cells was less than 20% after overnight incubation with 1,000 ng/ml of trimeric HuYON007-THA. When Ramos cells were incubated overnight in the presence of 1,000 ng/ml HuYON007-KH, the viability was nearly 100% in this assay. This result reconfirms that trimeric IgG antibodies of this invention can efficiently induce apoptosis of cells.

### Example 6

### Bispecific Trimeric Antibodies

Trimeric IgG antibodies of this invention can be produced in the single-chain Fv (scFv) format (Ahmad et al., Clin. Dev. Immunol. 2012:980250, 2012), for example, by modifying the expression vector pHuYON007-H in the following manner.

The transcription unit for the HuYON007 light chain, including the CMV promoter (CMV-P) and the polyadenylation site, is first removed in pHuYON007-H, and then the regions encoding VH, CH1 and hinge is replaced with an exon encoding, from 5′ to 3′, a signal peptide, mature HuYON007 VL, a flexible polypeptide linker, mature HuYON007 VH, a polypeptide linker, and the human gamma-1 hinge region (HuYON007.scFv-hinge). An Agel site is placed between the VH and hinge coding regions. The amino acid sequence of HuYON007.scFv-hinge, including the signal peptide, is MAWISLILSLLALSSGAISQTVVTQEPSFSVSPGGTVTLTCRSSSGAVTTSNFANWVQQTPGQAPRGLIGGTN NRAPGVPDRFSGSLLGNKAALTITGAQADDESDYYCALWYSNHWVFGGGTKLTVLGGGGSGGGGSGGGG SQVTLRESGPALVKPTQTLTLTCTFSGFSLSTSGMGVSWIRQPPGKALEWLAHIYWDDDKRYNPSLKSRLTIS KDTSKNQVVLTMTNMDPVDTATYYCTRRGEYGNFDYWGQGTLVTVSSTGGGEPKSCDKTHTCPPCP (SEQ ID NO:9). The mature polypeptide starts at position 20 in SEQ ID NO:9.

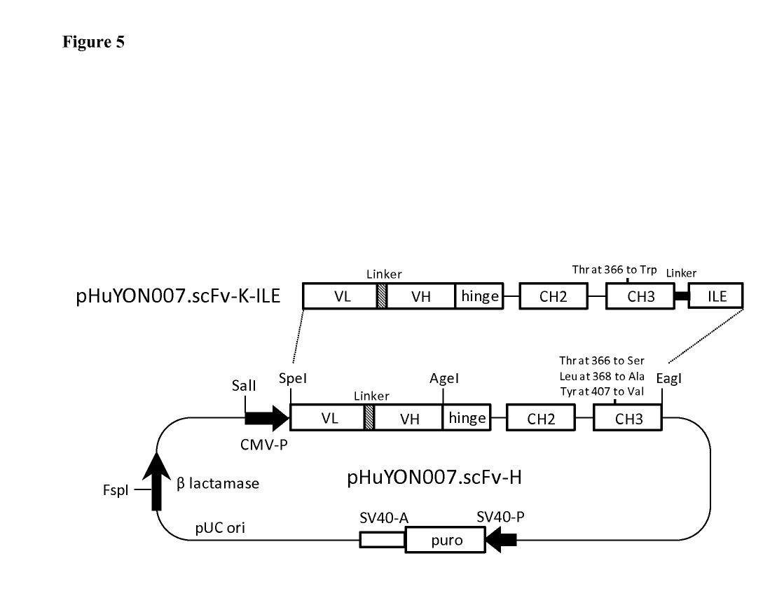

The schematic structure of the resultant plasmid, pHuYON007.scFv-H, is shown in FIG. 5. The amino acid sequence of mature HuYON007 scFv-Fc fusion protein encoded in pHuYON007.scFv-His QTVVTQEPSFSVSPGGTVTLTCRSSSGAVTTSNFANWVQQTPGQAPRGLIGGTNNRAPGVPDRFSGSLLGN KAALTITGAQADDESDYYCALWYSNHWVFGGGTKLTVLGGGGSGGGGSGGGGSQVTLRESGPALVKPTQT LTLTCTFSGFSLSTSGMGVSWIRQPPGKALEWLAHIYWDDDKRYNPSLKSRLTISKDTSKNQVVLTMTNMDP VDTATYYCTRRGEYGNFDYWGQGTLVTVSSTGGGEPKSCDKTHTCPPCPASTKGPSVFPLAPSSKSTSGGTA ALGCLVKDYFPEPVTVSWNSGALTSGVHTFPAVLQSSGLYSLSSVVTVPSSSLGTQTYICNVNHKPSNTKVDK KVEPKSCDKTHTCPPCPAPELLGGPSVFLFPPKPKDTLMISRTPEVTCVVVDVSHEDPEVKFNWYVDGVEVH NAKTKPREEQYNSTYRVVSVLTVLHQDWLNGKEYKCKVSNKALPAPIEKTISKAKGQPREPQVYTLPPSRDEL TKNQVSLSCAVKGFYPSDIAVEWESNGQPENNYKTTPPVLDSDGSFFLSSKLTVDKSRWQQGNVFSCSVMH EALHNHYTQKSLSLSPGK (SEQ ID NO:10).

The plasmid pHuYON007.scFv-His further modified by replacing the CH3 exon with the exon encoding the CH3 region and the isoleucine zipper of pHuYON007-K-ILE to generate pHuYON007.scFv-K-ILE (FIG. 5). The amino acid sequence of mature HuYON007 scFv-Fc fused to isoleucine zipper (HuYON007 scFv-Fc-ILE) encoded in pHuYON007.scFv-K-ILE is QTVVTQEPSFSVSPGGTVTLTCRSSSGAVTTSNFANWVQQTPGQAPRGLIGGTNNRAPGVPDRFSGSLLGN KAALTITGAQADDESDYYCALWYSNHWVFGGGTKLTVLGGGGSGGGGSGGGGSQVTLRESGPALVKPTQT LTLTCTFSGFSLSTSGMGVSWIRQPPGKALEWLAHIYWDDDKRYNPSLKSRLTISKDTSKNQVVLTMTNMDP VDTATYYCTRRGEYGNFDYWGQGTLVTVSSTGGGEPKSCDKTHTCPPCPASTKGPSVFPLAPSSKSTSGGTA ALGCLVKDYFPEPVTVSWNSGALTSGVHTFPAVLQSSGLYSLSSVVTVPSSSLGTQTYICNVNHKPSNTKVDK KVEPKSCDKTHTCPPCPAPELLGGPSVFLFPPKPKDTLMISRTPEVTCVVVDVSHEDPEVKFNWYVDGVEVH NAKTKPREEQYNSTYRVVSVLTVLHQDWLNGKEYKCKVSNKALPAPIEKTISKAKGQPREPQVYTLPPSRDEL TKNQVSLWCLVKGFYPSDIAVEWESNGQPENNYKTTPPVLDSDGSFFLYSKLTVDKSRWQQGNVFSCSVM HEALHNHYTQKSLSLSPGKSGGGSGGGSMKQIEDKIEEILSKIYHIENEIARIKKLIGERAG (SEQ ID NO:11).

The plasmid pHuYON007.scFv-H produces HuYON007 scFv-Fc(H) fusion proteins having the hole function of the knobs-into-holes structure for heterodimeric Fc-Fc interaction (Atwell et al., supra). The plasmid pHuYON007.scFv-K-ILE produces HuYON007 scFv-Fc(K)-ILE fusion proteins having the knob function of the knobs-into-holes structure for heterodimeric Fc-Fc interaction (Atwell et al., supra). When both pHuYON007.scFv-H and pHuYON007.scFv-K-ILE were simultaneously introduced into a cell, such as CHO-K1, HuYON007 scFv-Fc(H) fusion proteins expressed from pHuYON007.scFv-H and HuYON007 scFv-Fc(K)-ILE fusion proteins from pHuYON007.scFv-K-ILE form heterodimeric HuYON007.scFv antibodies. Furthermore, such hetero-dimeric scFv antibodies form trimeric hexavalent HuYON007.scFv antibodies due to homo-trimer formation by the isoleucine zipper.

For expression of bispecific trimeric scFv antibodies, the VH and VL coding regions in pHuYON007.scFv-H are first replaced respectively, for example, with the VH and VL coding regions of an antibody against human death receptor 5 (DR5; also called TRAIL receptor 1 and TNFRSF10B). Such resulting expression vector, named pADR5.scFv-H, produces anti-DR5 scFv-Fc fusion proteins with the hole function of the knobs-into-holes structure (ADR5 scFv-Fc(H)). When scFv antibodies are simultaneously expressed from pADR5.scFv-H and pHuYON007.scFv-K-ILE in a cell, ADR5 scFv-Fc(H) associates with HuYON007 scFv-Fc(K)-ILE to form bispecific antibodies, which further form trimers due to the presence of the isoleucine zipper at the caryboxyl terminus of HuYON007 scFv-Fc(K)-ILE. Such produced trimeric scFv antibodies bind to both DR4 and DR5.

### Example 7

### Trimeric Fc Fusion Proteins

The invention of this work is applicable to generation of trimeric Fc fusion proteins. For example, the SpeI-Agel fragment encoding the VL, linker and VH regions in pHuYON007.scFv-H (FIG. 5) is replaced with the SpeI-Agel fragment encoding the extracellular region of human TNF receptor type II (TNFR-II; also called CD120b and TNFRSF1b) to construct a new expression vector named pTNFR-Fc-H. Fusion proteins of the extracellular region of human TNFR-II to human gamma-1 Fc region having the hole function of the knobs-into-holes structure (Atwell et al., supra) (TNFR-Fc(H)) are produced from pTNFR-Fc-H in cells. The same SpeI-Agel fragment encoding the extracellular region of TNFR-II is also used for replacement of the SpeI-Agel fragment of pHuYON007-K-ILE to construct pTNFR-Fc-K-ILE. Fusion proteins of the extracellular region of human TNF-R-II to human gamma-1 Fc region having the knob function of the knobs-into-holes structure (Atwell et al., supra) further fused to the isoleucine zipper (TNFR-Fc(K)-ILE) are produced from pTNFR-Fc-K-ILE in cells. Simultaneous expression of TNFR-Fc(H) and TNFR-Fc(K)-ILE fusion proteins in cells produce trimeric hexavalent Fc fusion proteins each composed of three TNFR-Fc(H) polypeptides and three TNFR-Fc(K)-ILE polypeptides.

The SpeI-Agel fragment encoding the extracellular region of TNFR-II in pTNFR-Fc-K-ILE is further replaced with the SpeI-Agel fragment, for example, encoding the extracellular region of human IL-1 receptor type I (IL1RA; also called CD121A) to construct a new expression vector pOL1RA-Fc-K-ILE. Fusion proteins of the extracellular region of human IL1RA to human gamma-1 Fc region having the knob function of the knobs-into-holes structure (Atwell et al., supra) further fused to the isoleucine zipper (IL1RA-Fc(K)-ILE) are produced from pIL1RA-Fc-K-ILE in cells. Simultaneous expression of TNFR-Fc(H) and IL1RA-Fc(K)-ILE in cells produce trimeric hexavalent Fc fusion proteins composed of three TNFR-Fc(H) polypeptides and three IL1RA-Fc(K)-ILE polypeptides. Such trimeric Fc fusion proteins have bispecificity for ligand binding; one specific to TNF and another to IL-1.

### Example 8

### Multimeric IgG Antibodies and Fc Fusion Proteins

Multimeric IgG antibodies and Fc fusion proteins are produced in the same fashion as described in the previous Examples by replacing a trimerizing peptide in an expression vector, such as pHuYON007-K-ILE and pTNFR-Fc-K-ILE, with a multimerizing peptide (Grigoryan et al., Curr. Opin. Struct. Biol. 18:477-483, 2008; Lupas, Trends Biol. Sci. 21:375-382, 1996).

Tetrameric IgG antibodies are generated by replacing a trimerizing peptide in an expression vector, such as pHuYON007-K-ILE, with a tetramerizing peptide. Examples of tetramerizing peptides are tetrabrachion (Stetefeld et al., Naure Struc. Biol. 7:772-776, 2000), modified GCN4 leucine zipper (Harbury et al., Science 262:1401-1407, 1993), and Sendai virus phosphoprotein (Tarbouriech et al., Nature Struc. Biol. 7:777-781, 2000). Co-expression of such modified pHuYON007-K-ILE in which a tetramerizing peptide is linked to the carboxyl terminus of the CH3 domain (pHuYON007-K-Tet) with pHuYON007-H in a cell results in production of HuYON007 antibodies each including one HuYON007 heavy chain with the knob function expressed from pHuYON007-K-Tet, one HuYON007 heavy chain with the hole function from pHuYON007-H, and two HuYON007 light chains. Such produced HuYON007 antibodies further form tetramers due to homo-tetrameric association of the tetramerizing peptide linked to the carboxyl terminus of the CH3 region of the heavy chain produced from pHuYON007-K-Tet.

Co-expression of a modified pTNFR-Fc-K-ILE vector in which a tetramerizing peptide linked to the C-terminus of the CH3 domain (pTNFR-Fc-K-Tet) with pTNFR-Fc-H in a cell results in production of TNFR-Fc fusion proteins, each of which is composed of one TNFR-Fc fusion protein with the knob function expressed from pTNFR-Fc-K-Tet and one TNFR-Fc fusion protein with the hole function from pTNFR-Fc-H. Such hetero-dimeric TNFR-Fc fusion proteins further form tetramers due to homo-tetrameric association of the tetramer-forming polypeptide linked to the carboxyl terminus of the CH3 region of Fc fusion proteins produced from pTNFR-Fc-K-Tet.

Other types of multimeric IgG antibodies and Fc fusion proteins are produced in the same strategy as described above. For production of pentameric IgG antibodies and Fc fusion proteins, a pentamerizing peptide, for example, Trp-zipper protein (also called Trp-14; Liu et al., Proc. Natl. Acad. Sci. USA 101:16156-16161. 2004) and cartilage oligomeric matrix protein (COMP; Malashkevich et al., Science 274: 761-765, 1996), is used to replace a trimerizing peptide in an expression vector, such as pHuYON007-K-ILE and pTNFR-Fc-K-ILE. For hexameric IgG antibodies and Fc fusion proteins, a hexamerizing peptide, such as CC-Hex (Zaccai et al., Nature Chem. Biol. 7:935-941, 2011), is used for replacement.

### Example 9

### Trimeric Anti-OX40 IgG Antibody

OX40 (also called CD134 and TNFRSF4) is a member of the TNF receptor superfamily. The mouse hybridoma producing the anti-human OX40 monoclonal IgG1/kappa antibody OHX10 was generated at JN Biosciences (Mountain View, Calif.) using a mouse NS0 myeloma cell line expressing the extracellular region of recombinant human OX40 (SEQ ID NO:14) on the cell surface as an immunogen and following standard hybridoma techniques. The amino acid sequence of OHX10 VH and VL was determined by standard experimental procedures such as the method described by Tsurushita et al. (supra). The amino acid sequence of OHX10 VH, including the signal peptide sequence, is MGRLTSSFLLLIVPAYVLSQVTLKESGPGILQPSQTLSLTCSFSGFSLSTSGVGVGWIRQPSGKGLEWLAHIWW DDDKYYNTALKSGLTISKDTSKNQVFLKIASVDTADTATYYCARIDWDGIAYWGQGTLVTVSA (SEQ ID NO:15). The mature OHX10 VH starts at position 20 in SEQ ID NO:15. The CDR1, CDR2 and CDR3 amino acid sequences of OHX10 VH based on the definition of Ka bat et al. (supra) are TSGVGVG (SEQ ID NO:16), HIWWDDDKYYNTALKS (SEQ ID NO:17) and IDWDGIAY (SEQ ID NO:18), respectively. The amino acid sequence of OHX10 VL, including the signal peptide sequence, is MDFQVQIFSFLLISASVIMSRGQIVLSQSPAILSTSPGEKVTMTCRASSSVSYMHWYQEKPGSSPKPWIYATS NLASGVPARFSGSGSGTSYSLTISRVEAEDAATYYCQQWSSNPWTFGGGTKLEIK (SEQ ID NO:19). The mature OHX10 VL starts at position 23 in SEQ ID NO:19. The CDR1, CDR2 and CDR3 amino acid sequences of OHX10 VL based on the definition of Kabat et al. (supra) are RASSSVSYMH (SEQ ID NO:20), ATSNLAS (SEQ ID NO:21) and QQWSSNPWT (SEQ ID NO:22), respectively.

Humanization of OHX10 VH and VL was carried out as described in Tsurushita et al. (supra). The amino acid sequence of humanized OHX10 (HuOHX10) VH, including the signal peptide, is MGRLTSSFLLLIVPAYVLSQVTLRESGPALVKPTQJLTLTCTFSGFSLSTSGVGVGWIRQPPGKALEWLAHIW WDDDKYYNTALKSGLTISKDTSKNQVVLTMTNMDPVDTATYYCARIDWDGIAYWGQGTLVTVSS (SEQ ID NO:23). The mature HuOHX10 VH sequence starts at position 20 in SEQ ID NO:23. The amino acid sequence of HuOHX10 VL, including the signal peptide, is MDFQVQIFSFLLISASVIMSRGEIVLTQSPATLSLSPGERATLSCRASSSVSYMHWYQQKPGQAPRPWIYATS NLASGIPARFSGSGSGTDYTLTISSLEPEDFAVYYCQQWSSNPWTFGGGTKVEIK (SEQ ID NO:24). The mature HuOHX10 VL sequence starts at position 23 in SEQ ID NO:24.

A gene encoding HuOHX10 VH (SEQ ID NO:25) was synthesized as an exon including a splice donor signal at the 3′ end of the coding region, an SpeI site at the 5′ end of the fragment, and a HindIII site at the 3′ end of the fragment. A gene encoding HuOHX10 VL (SEQ ID NO:26) was synthesized as an exon including a splice donor signal at the 3′ end of the coding region, an NheI site at the 5′ end of the fragment, and an EcoRI site at the 3′ end of the fragment. For expression of trimeric HuOHX10 IgG antibodies, two mammalian expression vectors, pHuOHX10-K-ILE and pHuOHX10-H, were constructed. The expression vectors pHuOHX10-K-ILE and pHuOHX10-H have a structure similar to pHuYON007-K-ILE and pHuYON007-H (FIG. 1), respectively, except that (a) the HuYON007 VH gene was replaced by the HuOHX10 VH gene between the SpeI and HindIII sites, (b) the HuYON007 VL gene was replaced by the HuOHX10 VL gene between the NheI and EcoRI sites, and (c) the coding region of the human lambda constant region was replaced by the coding region of the human kappa constant region, in both pHuOHX10-K-ILE and pHuOHX10-H.

Light chains encoded in pHuOHX10-K-ILE and pHuOHX10-H (HuOHX10 light chains) are identical to each other in their amino acid sequence. Heavy chains expressed from pHuOHX10-K-ILE and pHuOHX10-H preferentially form Fc-to-Fc heterodimeric molecules by the knobs-into-holes mechanism (Atwell, supra). When heavy and light chains from pHuOHX10-K-ILE and pHuOHX10-H are expressed simultaneously in cells, HuOHX10 antibodies each composed of one heavy chain from pHuOHX10-K-ILE, one heavy chain from pHuOHX10-H, and two HuOHX10 light chains (HuOHX10-THB; schematically illustrated as a monomer in FIG. 2B), are produced. Furthermore, HuOHX10-THB antibodies form trimers due to homo-trimeric association of the isoleucine zipper fused to the carboxyl terminus of the heavy chain produced from pHuOHX10-K-ILE (Harbury et al., supra). The structure of trimeric HuOHX10-THB is schematically illustrated in FIG. 2C.

Another vector for expression of HuOHX10 in the human IgG1/kappa form (HuOHX10-IgG1) was also constructed. The resulting expression vector, pHuOHX10-IgG1, has a structure similar to pHuYON007 (FIG. 1) except that (a) the HuYON007 VH exon was replaced by the HuOHX10 VH exon between the SpeI and HindIII sites, (b) the HuYON007 VL exon was replaced by the HuOHX10 VL exon between the NheI and EcoRI sites, and (c) the coding region of the human lambda constant region was replaced by the coding region of the human kappa constant region.

The expression vectors pHuOHX10-H and pHuOHX10-K-ILE were introduced together into the chromosomes of a Chinese hamster ovary cell line CHO-K1 (ATCC, Manassas, Va.) to obtain cell lines stably producing HuOHX10-THB. Separately, the expression vector pHuOHX10-IgG1 was transfected into CHO-K1 cells to obtain cell lines producing HuOHX10-IgG1. Stable transfection into CHO-K1 cells was carried out as described above. Expression of HuOHX10 antibodies was measured by sandwich ELISA as described above, except that bound antibodies were detected using HRP-conjugated goat anti-human kappa chain polyclonal antibody. CHO-K1 stable transfectants producing each of HuOHX10-THB and HuOHX10-IgG1 were expanded in SFM4CHO media. HuOHX10-THB and HuOHX10-IgG1 antibodies were purified by protein A affinity chromatography as described above. HuOHX10-THB trimer was obtained by further fractionation using a Superose 6 size exclusion column as described above.

Purified HuOHX10-IgG1 and trimeric HuOHX10-THB showed specific binding to human OX40 by flow cytometry using OX40-expressing cells. SDS-PAGE analysis under denaturing conditions indicated that purified trimeric HuOHX10-THB was composed of three polypeptides. The largest polypeptide of approximately 55 kDa corresponds to heavy chains expressed from pHuOHX10-K-ILE. The second largest polypeptide of approximately 50 kDa corresponds to heavy chains expressed from pHuOHX10-H. The intensity of 55 kDa and 50 kDa bands was similar to each other. The smallest polypeptide of approximately 25 kDa corresponds to light chains expressed from both pHuOHX10-K-ILE and pHuOHX10-H. HuOHX10-IgG1 was composed of two polypeptides in SDS-PAGE analysis under denaturing conditions. The larger polypeptide of approximately 50 kDa corresponds to heavy chains and the smaller polypeptide of approximately 25 kDa corresponds to light chains.

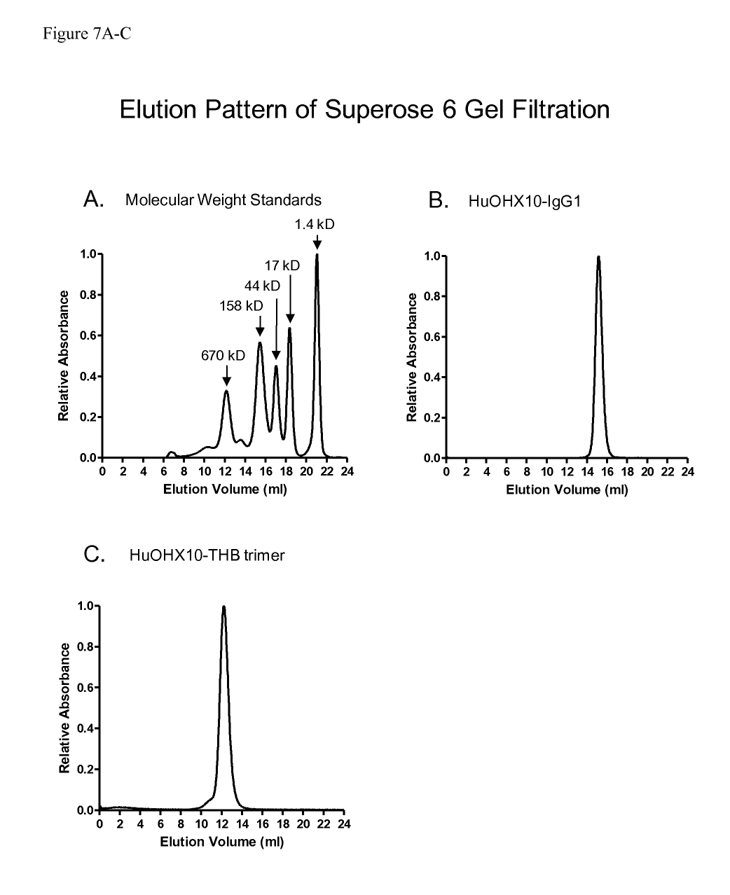

The molecular size of purified HuOHX10-IgG1 and trimeric HuOHX10-THB in the native form was analyzed by gel filtration using the AKTA Basic FPLC system with a Superose 6 10/300 GL column which has a separation range from 5 to 5,000 kilo Dalton (kDa) of globular proteins (GE Healthcare, Indianapolis, Ind.). PBS was used as elution buffer. When compared to the elution pattern of molecular size markers (FIG. 7A), the size of HuOHX10-IgG1 was estimated to be approximately 160 kDa (FIG. 7B), which corresponds to the size of a monomeric human IgG1 antibody composed of two heavy and two light chains. For HuOHX10-THB trimer, a single dominant peak was observed at 12.2 ml of elution (FIG. 7C), which is an approximate location of the elution of trimeric IgG when compared to the elution pattern of size markers (FIG. 7A).

To examine the costimulatory activity of anti-OX40 antibodies, a human cutaneous T lymphocyte cell line HuT-78 (Cat No. TIB-161, ATCC, Manassas, Va.) stably expressing recombinant human OX40 on the surface (HuT-78/OX40) was generated at JN Biosciences. Cross-linking of OX40 on the surface of HuT-78/OX40 cells is known to increase IL-2 production when the cells are simultaneously treated with anti-CD3 and anti-CD28 antibodies (US2008002498). Cross-linking of OX40 also increases IL-2 production in HuT-78/OX40 cells when CD3 molecules alone are simultaneously cross-linked.

One hundred thousand HuT-78/OX40 cells in 0.2 ml of RPMI-1640 medium containing 10% FBS were placed in each well of a 96-well plate in the presence of 1 μg/ml mouse anti-human CD3 monoclonal antibody (OKT3, Cat. No. 70-0030, Tonbo Biosciences, San Diego, Calif.), 5 μg/ml goat anti-mouse IgG polyclonal antibody (Cat. No. 115-005-071, Jackson ImmunoResearch Laboratories, West Grove, Pa.), and 1 μg/ml of a test anti-OX40 antibody as specified below. As a background control, HuT-78/OX40 cells were grown without any antibodies. After 72 hours of incubation at 37° C. in a 7.5% CO₂ incubator, IL-2 concentration in culture supernatants was measured by ELISA (Human IL-2 ELISA MAX™ Standard Kit, Cat No. 431801, BioLegend, San Diego, Calif.). When HuT-78/OX40 cells were incubated without any antibodies (thus no CD3 cross-linking), IL-2 concentration in the culture supernatants was less than 78 pg/ml. When HuT-78/OX40 cells were incubated with OKT3 and goat anti-mouse IgG antibody (thus CD3 molecules are cross-linked), IL-2 concentration was 103 pg/ml with no anti-OHX10 antibodies, 103 pg/ml with HuOHX10-IgG1, and 627 pg/ml with trimeric HuOHX10-THB. Thus, the trimeric anti-OX40 IgG antibody of this invention induced IL-2 expression in T cells via cross-linking of OX40 molecules on the surface much more efficiently than anti-OX40 IgG antibodies did.

### Example 10

### Trimeric Anti-CD40 IgG Antibody

CD40 (also called TNFRSF5) is a member of the TNF receptor superfamily. The mouse hybridoma producing the anti-human CD40 monoclonal IgG1/kappa antibody 11D1 was generated at JN Biosciences (Mountain View, Calif.) using the extracellular region of human CD40 fused to the Fc region of human gamma-1 heavy chain (CD40-Fc) (SEQ ID NO:27) as an immunogen and following standard hybridoma techniques. The amino acid sequence of 11D1 VH and VL was determined by standard experimental procedures such as the method described by Tsurushita et al. (supra). The amino acid sequence of 11D1 VH, including the signal peptide sequence, is MDIRLSLAFLVLFIKGVQCEVQLVESGGGLVQPGRSMKLSCAASGFTFSYFPMAWVRQAPTKGLEWVATIST SGGNIYYRDSVKGRFTISRDNAKSTLYLQMNSLRSEDTATYYCTRDTAPYYFDYWGQGVMVTVSS (SEQ ID NO:28). The mature 11D1 VH starts at position 20 in SEQ ID NO:28. The CDR1, CDR2 and CDR3 amino acid sequences of 11D1 VH based on the definition of Kabat et al. (supra) are YFPMA (SEQ ID NO:29), TISTSGGNIYYRDSVKG (SEQ ID NO:30) and DTAPYYFDY (SEQ ID NO:31), respectively. The amino acid sequence of 11D1 VL, including the signal peptide sequence, is MRAHAQFLGLLLLWFPGARCDIQMTQSPSSISVSLGDRFTITCRASQDIGNYLNWYQQKPEKSPKLMIYRAT NLEDGVPSRFSGSRSGSDYSLTINSLESEDTGFYFCVQHKQYPLTFGSGTKLEIK (SEQ ID NO:32). The mature 11D1 VL starts at position 21 in SEQ ID NO:32. The CDR1, CDR2 and CDR3 amino acid sequences of 11D1 VL based on the definition of Kabat et al. (supra) are RASQDIGNYLN (SEQ ID NO:33), RATNLED (SEQ ID NO:34) and VQHKQYPLT (SEQ ID NO:35), respectively.

Humanization of 11D1 VH and VL was carried out as described in Tsurushita et al. (supra). The amino acid sequence of humanized 11D1 (Hu11D1) VH, including the signal peptide, is MDIRLSLAFLVLFIAGVQCEVQLVESGGGLVQPGGSLRLSCAASGFTFSYFPMAWVRQAPGKGLEWVATIST SGGNIYYRDSVKGRFTISRDNAKNSLYLQMNSLRAEDTAVYYCTRDTAPYYFDYWGQGTMVTVSS (SEQ ID NO:36). The mature Hu11D1 VH sequence starts at position 20 in SEQ ID NO:36. The amino acid sequence of Hu11D1 VL, including the signal peptide, is MRAHAQFLGLLLLWFPGARCDIQMTQSPSSLSASVGDRVTITCRASQDIGNYLNWYQQKPGKAPKLLIYRAT NLEDGVPSRFSGSGSGTDYTLTISSLQPEDFATYYCVQHKQYPLTFGGGTKVEIK (SEQ ID NO:37). The mature Hu11D1 VL sequence starts at position 21 in SEQ ID NO:37.

Each of the genes encoding Hu11D1 VH (SEQ ID NO:38) and Hu11D1 VL (SEQ ID NO:39) was synthesized as described in Example 9. For expression of trimeric Hu11D1 IgG antibodies, two mammalian expression vectors, pHu11D1-K-ILE and pHu11D1-H, were constructed. The expression vectors pHu11D1-K-ILE and pHu11D1-H have a structure similar to pHuOHX10-K-ILE and pHuOHX10-H described in Example 9), respectively, except that (a) the HuOHX10 VH gene was replaced by the Hu11D1 VH exon between the SpeI and HindIII sites and (b) the HuOHX10 VL exon was substituted for the Hu11D1 VL exon between the NheI and EcoRI sites.

Light chains encoded in pHu11D1-K-ILE and pHu11D1-H (Hu11D1 light chains) are identical to each other in their amino acid sequence. Heavy chains expressed from pHu11D1-K-ILE and pHu11D1-H preferentially form Fc-to-Fc heterodimeric molecules (Atwell, supra). When heavy and light chains from pHu11D1-K-ILE and pHu11D1-H are expressed simultaneously in cells, Hu11D1 antibodies each composed of one heavy chain from pHu11D1-K-ILE, one heavy chain from pHu11D1-H, and two Hu11D1 light chains (Hu11D1-THB; schematically illustrated as a monomer in FIG. 2B), are produced. Furthermore, Hu11D1-THB antibodies form trimers due to homo-trimeric association of the isoleucine zipper fused to the carboxyl terminus of heavy chains produced from pHu11D1-K-ILE (Harbury et al., supra).

The expression vectors pHu11D1-K-ILE and pHu11D1-H were simultaneously introduced into the chromosome of a mouse myeloma cell line NS0 (European Collection of Animal Cell Cultures, Salisbury, Wiltshire, UK) to obtain cell lines stably producing Hu11D1-THB antibodies. NS0 cells were grown in DME medium containing 10% fetal bovine serum (FBS; HyClone, Logan, Utah) at 37° C. in a 7.5% CO2 incubator. Stable transfection into NS0 cells was carried out by electroporation as described in Bebbington et al. (Bio/Technology 10: 169-175, 1992). Before transfection, two expression vectors were linearized using Fspl. In a typical experiment, approximately 10⁷ cells were transfected with 20 μg of linearized plasmid, suspended in DME medium containing 10% FBS, and plated into several 96-well plates. After 48 hr, selection media (DME medium containing 10% FBS and 3 μg/ml puromycin) was applied. Expression of Hu11D1-THB in culture supernatants was measured by sandwich ELISA as described in Example 9. NS0 stable transfectants producing a high level of Hu11D1-THB were expanded in serum-free media using Hybridoma SFM (Invitrogen). Hu11D1-THB antibodies were purified using a protein A affinity column. Hu11D1-THB timer was obtained by further fractionation using a Superose 6 size exclusion column as described above.

Another vector for expression of Hu11D1 in the human IgG1/kappa form (Hu11D1-IgG1) was also constructed. The resulting expression vector, pHu11D1-IgG1, has a structure identical to pHuOHX10-IgG1 except that (a) the HuOHX10 VH gene was replaced by the Hu11D1 VH gene between the SpeI and HindIII sites and (b) the HuOHX10 VL gene was replaced by the Hu11D1 VL gene between the NheI and EcoRI sites. The expression vector pHu11D1-IgG1 was introduced into the chromosomes of a Chinese hamster ovary cell line CHO-K1 (ATCC, Manassas, Va.) to obtain cell lines stably producing Hu11D1-IgG1. Stable transfection into CHO-K1 cells, selection of high antibody producers, expansion in serum-free media, and purification of Hu11D1-IgG1 antibodies using a Protein A column were carried out as described above.

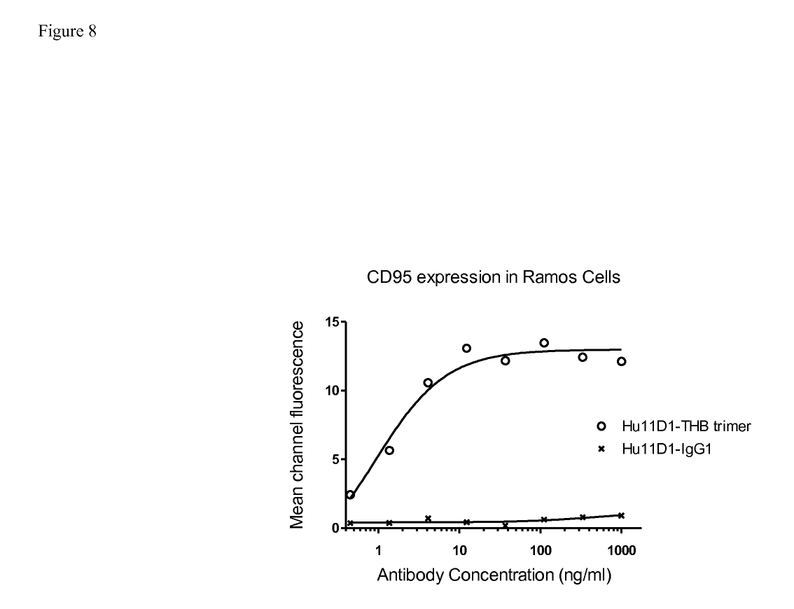

The human Burkitt's B lymphoma cell line Ramos expresses CD40 on the surface (Henriquez et al., J. Immunol. 162:3298-3307, 1999). Cross-linking of CD40 on the surface of Ramos cells with soluble trimeric CD40 ligand (also called CD40L, CD154 and TNFSF5) is known to induce elevated expression of CD95 (Henriquez et al., supra). In order to examine the ability of anti-CD40 antibodies to activate antigen-presenting cells, purified Hu11D1-THB trimer and Hu11D1-IgG1 antibodies were individually incubated at various concentrations, starting at 1000 ng/ml and three-fold serial dilutions, with Ramos cells in DME media containing 10% FBS at 37° C. for 48 hr in a 7.5% CO₂ incubator. Ramos cells were then stained with PE-labeled mouse anti-CD95 monoclonal antibody (Cat. No. 305608, BioLegend, San Diego, Calif.) and analyzed by flow cytometry to measure the expression level of CD95 on the cell surface. FIG. 8 shows the plot of geometric mean channel fluorescence (MCF) of Ramos cells (y-axis) at each antibody concentration (x-axis). As shown in FIG. 8, Hu11D1-IgG1 failed to significantly induce the expression of CD95 on Ramos cells even at 1000 ng/ml. On the other hand, the ability of the trimeric anti-CD40 IgG antibody of this invention (Hu11D1-THB trimer) to induce CD95 expression was clearly observed at 4.1 ng/ml and reached the maximal level at approximately 10 ng/ml. As an example of the data, the MCF values of Ramos cells grown in the presence of no antibody, 12.3 ng/ml of Hu11D1-IgG1, and 12.3 ng/ml of Hu11D1-THB trimer were 2.6, 3.1 and 15.7, respectively.

### Example 11

### Use of CD40 Ligand for Formation of Trimeric IgG Antibodies

To obtain another example of trimeric IgG antibodies of this invention, the isoleucine zipper-coding region in pHuYON007-K-ILE was replaced by a DNA fragment encoding the extracellular region of human CD40L, a member of the TNF superfamily, which is known to form homo-trimers (Bodmer et al., Trends Biochem. Sci. 27:19-26, 2002). In addition, an amino acid at position 143 of CD40L was changed from Lys to Thr (K143T) to eliminate its interaction with CD40 without losing its ability to form a trimer (An et al., J. Biol. Chem. 286:11226-11235, 2011). The amino acid sequence of the extracellular region of human CD40L with the K143T mutation is GDQNPQIAAHVISEASSKTTSVLQWAETGYYTMSNNLVTLENGKQLTVKRQGLYYIYAQVTFCSNREASSQA PFIASLCLKSPGRFERILLRAANTHSSAKPCGQQSIHLGGVFELQPGASVFVNVTDPSQVSHGTGFTSFGLLKL (SEQ ID NO: 40). The resultant expression vector was named pHuYON007-K-CD40L. In this construct, the carboxyl terminal lysine residue in the CH3 domain of the gamma heavy chain was also removed. The amino acid sequence of the modified heavy chain constant region in pHuYON007-K-CD40L is ASTKGPSVFPLAPSSKSTSGGTAALGCLVKDYFPEPVTVSWNSGALTSGVHTFPAVLQSSGLYSLSSVVTVPSS SLGTQTYICNVNHKPSNTKVDKKVEPKSCDKTHTCPPCPAPELLGGPSVFLFPPKPKDTLMISRTPEVTCVVVD VSHEDPEVKFNWYVDGVEVHNAKTKPREEQYNSTYRVVSVLTVLHQDWLNGKEYKCKVSNKALPAPIEKTIS KAKGQPREPQVYTLPPSRDELTKNQVSLWCLVKGFYPSDIAVEWESNGQPENNYKTTPPVLDSDGSFFLYSK LTVDKSRWQQGNVFSCSVMHEALHNHYTQKSLSLSPGSGGGSGGGSGDQNPQIAAHVISEASSKTTSVLQ WAETGYYTMSNNLVTLENGKOLTVKROGLYYIYAQVTFCSNREASSQAPFIASLCLKSPGRFERILLRAANTHS SAKPCGQQSIHLGGVFELQPGASVFVNVTDPSQVSHGTGFTSFGLLKL (SEQ ID NO:41).

The two expression vectors, pHuYON007-H and pHuYON007-K-CD40L, were transfected together into HEK293 cells using Lipofectamine 2000 reagent as described in Example 2. Light chains encoded in pHuYON007-H and pHuYON007-K-CD40L (HuYON007 light chains) are identical to each other in their amino acid sequence. Heavy chains expressed from pHuYON007-K-CD40L and pHuYON007-H preferentially form Fc-to-Fc heterodimeric molecules (Atwell, supra). When heavy and light chains from pHuYON007-K-CD40L and pHuYON007-H are expressed simultaneously in cells, HuYON007 antibodies each composed of one heavy chain from pHuYON007-K-CD40L, one heavy chain from pHuYON007-H, and two HuYON007 light chains (HuYON007-THF; schematically illustrated as a monomer in FIG. 2B), are produced. Furthermore, HuYON007-THF antibodies form trimers due to homo-trimeric association of CD40L fused to the carboxyl terminus of heavy chains produced from pHuYON007-K-CD40L (schematically illustrated in FIG. 2C) (Bodmer et al., supra).

Culture supernatants of HEK293 cells transfected with pHuYON007-K-CD40L and pHuYON007-H were fractionated by gel filtration using a Superose 6 10/300 GL column and the presence of HuYON007 antibodies in each fraction was analyzed by sandwich ELISA as described in Example 2. HuYON007-THF antibodies were eluted at fractions corresponding to roughly 670 kDa, which is consistent with the expected size of HuYON007-THF trimers.

### Example 12

### Dimeric IgG Antibodies

A new vector for expression of dimeric tetravalent IgG antibodies was constructed by replacing the coding region of the isoleucine zipper in pHuYON007-K-ILE with a DNA fragment encoding a polypeptide known as a leucine zipper (SEQ ID NO:42) which is capable of forming homo-dimers (Harbury et al., Science 262:1401-1407, 1993). The resulting expression vector was named pHuYON007-K-LEU. The amino acid sequence of the modified heavy chain constant region encoded in pHuYON007-K-LEU is ASTKGPSVFPLAPSSKSTSGGTAALGCLVKDYFPEPVTVSWNSGALTSGVHTFPAVLQSSGLYSLSSVVTVPSS SLGTQTYICNVNHKPSNTKVDKKVEPKSCDKTHTCPPCPAPELLGGPSVFLFPPKPKDTLMISRTPEVTCVVVD VSHEDPEVKFNWYVDGVEVHNAKTKPREEQYNSTYRVVSVLTVLHQDWLNGKEYKCKVSNKALPAPIEKTIS KAKGQPREPQVYTLPPSRDELTKNQVSLWCLVKGFYPSDIAVEWESNGQPENNYKTTPPVLDSDGSFFLYSK LTVDKSRWQQGNVFSCSVMHEALHNHYTQKSLSLSPGKSGGGSGGGSHMKQLEDKVEELLSKNYHLENEV ARLKKLVGERAG (SEQ ID NO:43). Light chains encoded in pHuYON007-K-LEU and pHuYON007-H are identical to each other in their amino acid sequence.

The expression vectors pHuYON007-H and pHuYON007-K-LEU were introduced together into the chromosomes of a Chinese hamster ovary cell line CHO-K1 (ATCC, Manassas, Va.) by the transfection method described above to obtain cell lines stably producing HuYON007-THD, which is composed in each monomer of one heavy chain expressed from pHuYON007-H, one heavy chain expressed from pHuYON007-K-Leu, and two light chains expressed from pHuYON007-H and pHuYON007-K-Leu. HuYON007-THD antibodies form dimers due to homo-dimeric association of the leucine zipper fused to the carboxyl terminus of the heavy chain produced from pHuYON007-K-LEU (Harbury et al., supra). Expression of HuYON007 antibodies in CHO-K1 stable transfectants was measured by sandwich ELISA as described above. CHO-K1 stable transfectants producing HuYON007-THD were expanded in SFM4CHO media. Purification of HuYON007-THD from culture supernatants with Protein A and Superose 6 columns was carried out as described above.

For expression of HuYON007 IgG1 monomers, pHuYON007 was stably transfected into CHO-K1 as described above. CHO-K1 stable transfectants producing HuYON007 IgG1 were expanded in SFM4CHO media. Purification of HuYON007 IgG1 from culture supernatants with Protein A was carried out as described above.

SDS-PAGE analysis under denaturing conditions indicated that purified HuYON007-THD was composed of three polypeptides. The largest polypeptide of approximately 55 kDa corresponds to heavy chains expressed from pHuYON007-K-LEU. The second largest polypeptide of approximately 50 kDa corresponds to heavy chains expressed from pHuYON007-H. The intensity of 55 kDa and 50 kDa bands was similar to each other. The smallest polypeptide of approximately 25 kDa corresponds to light chains expressed from both pHuYON007-K-LEU and pHuYON007-H.

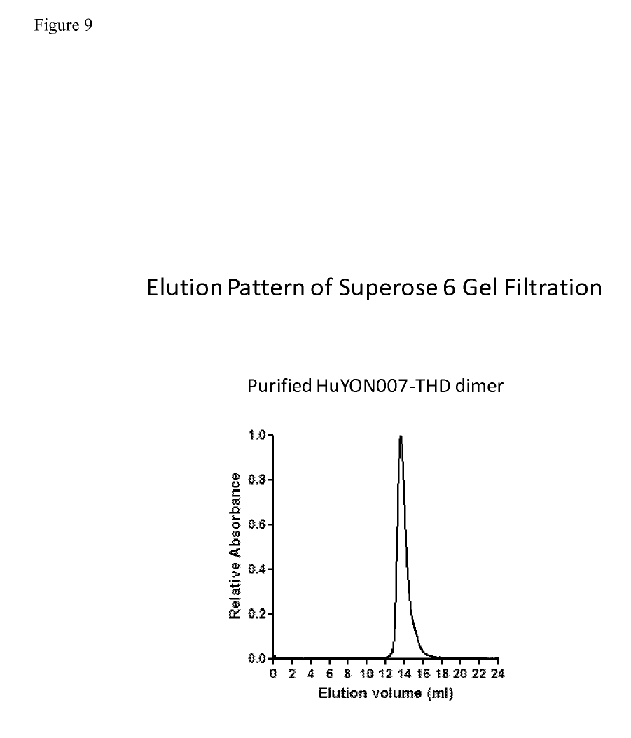

The molecular size of purified HuYON007-THD in the native form was analyzed by gel filtration using a Superose 6 column as described in Example 2. As shown in FIG. 9, purified HuYON007-THD showed a peak of elution at 13.6 ml, which corresponds to a molecular size of roughly 400 kDa based on the elution pattern of molecular markers (FIG. 3A). This is consistent with the expected size of dimeric HuYON007-THD antibody. Under the same elution condition, HuYON007-KH (monomeric IgG) and HuYON007-THB (trimeric IgG) showed a peak of elution at 15.6 ml and 12.5 ml, respectively (FIGS. 3B and C).

To assess the ability to induce DR4-mediated apoptosis, the human Burkett's lymphoma cell line Ramos, which expresses DR4 on the cell surface, was grown in the presence of HuYON007 IgG1 (monomeric IgG), HuYON007-THD (dimeric IgG) or HuYON007-THB (trimeric IgG) in duplicate. After overnight incubation, cell viability was measured with alamar Blue (Invitrogen) according to the manufacturer's protocol. Percent cell viability was calculated by normalizing the absorbance value in the presence of each test antibody to that in the absence of test antibodies (100% viability). The absorbance value with no cells was used as background (zero % viability). The viability of Ramos cells was 78.3% for HuYON007 IgG1, 29.2% for HuYON007-THD, and 5.6% for HuYON007-THB when the antibody concentration was 111 ng/ml. The viability was 81.4% for HuYON007-KH, 51.8% for HuYON007-THD, and 10.3% for HuYON007-THB when the antibody concentration was 12.3 ng/ml. Although HuYON007-THD (dimeric IgG) was not as potent as HuYON007-THB (trimeric IgG), HuYON007-THD was more potent than HuYON007 (monomeric IgG) for induction of apoptosis.

### Example 13

### Tetrameric IgG Antibodies

A new vector for expression of tetrameric octavalent IgG antibodies was constructed by replacing the coding region of the isoleucine zipper in pHuYON007-K-ILE with a DNA fragment encoding a polypeptide derived from the GCN4 leucine zipper capable of forming homo-tetramers (referred to as a tetra zipper herein) (SEQ ID NO:44) (Harbury et al., Science 262:1401-1407, 1993). The resulting expression vector was named pHuYON007-K-Tetra. The amino acid sequence of the heavy chain constant region encoded in pHuYON007-K-Tetra is ASTKGPSVFPLAPSSKSTSGGTAALGCLVKDYFPEPVTVSWNSGALTSGVHTFPAVLQSSGLYSLSSVVTVPSS SLGTQTYICNVNHKPSNTKVDKKVEPKSCDKTHTCPPCPAPELLGGPSVFLFPPKPKDTLMISRTPEVTCVVVD VSHEDPEVKFNWYVDGVEVHNAKTKPREEQYNSTYRVVSVLTVLHQDWLNGKEYKCKVSNKALPAPIEKTIS KAKGQPREPQVYTLPPSRDELTKNQVSLWCLVKGFYPSDIAVEWESNGQPENNYKTTPPVLDSDGSFFLYSK LTVDKSRWQQGNVFSCSVMHEALHNHYTQKSLSLSPGKSGGGSGGGSHMKQIEDKLEEILSKLYHIENELAR IKKLLGERAG (SEQ ID NO:45). Light chains encoded in pHuYON007-K-Tetra and pHuYON007-H are identical to each other in their amino acid sequence.

The expression vectors pHuYON007-H and pHuYON007-K-Tetra were cotransfected into CHO-K1 (ATCC, Manassas, Va.) by the transfection method described above to obtain cell lines stably producing HuYON007-THG, which is composed in each monomer of one heavy chain expressed from pHuYON007-H, one heavy chain expressed from pHuYON007-K-Tetra, and two light chains expressed from pHuYON007-H and pHuYON007-K-Tetra. HuYON007-THG antibodies form tetramers due to homo-tetrameric association of the tetra zipper fused to the carboxyl terminus of the heavy chain produced from pHuYON007-K-Tetra (Harbury et al., supra).

Purification of HuYON007 antibodies using protein A and Superose 6 size exclusion columns was conducted as described above. SDS-PAGE analysis under denaturing conditions indicated that purified HuYON007-THG was composed of three polypeptides. The largest polypeptide of approximately 55 kDa corresponds to heavy chains expressed from pHuYON007-K-Tetra. The second largest polypeptide of approximately 50 kDa corresponds to heavy chains expressed from pHuYON007-H. The intensity of 55 kDa and 50 kDa bands was similar to each other. The smallest polypeptide of approximately 25 kDa corresponds to light chains expressed from both pHuYON007-K-Tetra and pHuYON007-H.

The molecular size of purified HuYON007-THG in the native form was analyzed by gel filtration using a Superose 6 column as described in Example 2. Purified HuYON007-THG showed a peak of elution at 11.8 ml, which corresponds to a molecular size of roughly 800 kDa based on the elution pattern of molecular markers. This is consistent with the expected size of tetrameric HuYON007-THG antibody. Under the same elution condition, HuYON007-KH (monomeric IgG; FIG. 3B), HuYON007-THD (dimeric IgG; FIG. 8) and HuYON007-THB (trimeric IgG; FIG. 3C) showed a peak of elution at 15.6 ml, 13.6 ml, and 12.5 ml, respectively.

The ability of HuYON007-THG to induce DR4-mediated apoptosis of Ramos was examined as described in Example 12. The viability of Ramos cells was nearly 100% for HuYON007 IgG1 and approximately 6% for HuYON007-THG when the antibody concentration was 6.2 ng/ml, thus indicating that tetrameric HuYON007-THG can induce DR4-mediated apoptosis more efficiently than monomeric HuYON007 IgG1.

### Example 14

### Bispecific Trimeric Anti-DR4/DR5 IgG Antibodies

Humanized anti-human death receptor 5 (DR5; also called TRAIL receptor 2, TNFRSF10B, CD262) IgG1/kappa monoclonal antibody HuGOH729S, which was generated at JN Biosciences using standard hybridoma and humanization technologies, was reported previously (US20140037621). The mouse hybridoma producing the parental antibody of HuGOH729S was generated using the extracellular region of human DR5 fused to the Fc region of human gamma-1 heavy chain (DR5-Fc) (SEQ ID NO:46) as an immunogen. The amino acid sequence of HuGOH729S VH, including the signal peptide sequence, is MEWCWVFLFLLSVTAGVHSQVQLVQSGAEVKKPGASVKVSCKASGYTFTDYIIHWVRQAPGQGLEWIGW FYPGNNNIKSNEKFKDRVTLTADTSTSTVYMELSSLRSEDTAVYYCARNEDNYGNFFGYWGQGTLVTVSS (SEQ ID NO:47). The mature HuGOH729S VH starts at position 20 in SEQ ID NO:47. The amino acid sequence of HuGOH729S VL, including the signal peptide sequence, is MESQIQAFVFVFLWLSGVDGDIQMTQSPSSLSASVGDRVTITCKASQDVNTAAAWYQQKPGKAPKLLIYW ASTRHTGVPSRFSGSGSGTDYTLTISSLQPEDFATYYCQQHYSTPYTFGQGTKLEIK (SEQ ID NO:48). The mature HuGOH729S VL starts at position 21 in SEQ ID NO:48.

The HuGOH729S VL and VH coding regions were cloned into pHuYON007.scFv-H (Example 6) to replace the HuYON007 VL and VH coding regions, respectively, for expression of HuGOH729S scFv-Fc fusion proteins with the hole mutation in the Fc region (SEQ ID NO:49). The resulting plasmid was named pHuGOH729S.scFv-H. Similarly, the HuGOH729S VL and VH coding regions were cloned into pHuYON007.scFv-K-ILE (Example 6) to replace the HuYON007 VL and VH coding regions, respectively, for expression of HuGOH729S scFv fused to the Fc region with the knob mutation and further to the isoleucine zipper (SEQ ID NO:50). The resulting plasmid was named pHuGOH729S.scFv-K-ILE.

Transient expression of single-chain Fv antibodies in HEK293 cells was carried out as described above with the following four combinations of the expression vectors: (1) pHuYON007.scFv-K-ILE and pHuYON007.scFv-H, producing 007/007 scFv antibodies (2) pHuGOH729S.scFv-K-ILE and pHuGOH729S.scFv-H, producing 729/729 scFv antibodies (3) pHuYON007.scFv-K-ILE and pHuGOH729S.scFv-H, producing 007/729 antibodies, and (4) pHuGOH729S.scFv-K-ILE and pHuYON007.scFv-H, producing 729/007 antibodies.

Bispecific binding of these four antibodies to DR4 and DR5 was examined by ELISA in the following format. Wells of a microtitre plate were coated with DR4-Fc (SEQ ID NO:1). After blocking the wells with Blocking Buffer, appropriately diluted culture supernatants of HEK293 cells were applied to the wells and incubated for 1 hr at room temperature. After washing wells with Wash Buffer, recombinant human DR5 extracellular region fused at the C-terminus to the human X2 constant region (DR5-Cλ; SEQ ID NO:51) in ELISA Buffer was applied to the wells. After incubating the ELISA plate for 30 min at room temperature and washing the wells with Wash Buffer, bound DR5-Cλ was detected by HRP-conjugated goat anti-human X chain polyclonal antibody. Color development was initiated by adding ABTS substrate and stopped with 2% oxalic acid. Absorbance was read at 405 nm. In this format of ELISA, strong signals, which indicate the presence of bispecific trimeric IgG antibodies that can bind to both DR4 and DR5, were observed only for the 007/729 and 729/007 antibodies. Neither 007/007 nor 729/729 antibodies produced any significant ELISA signals.

The presence of bispecific anti-DR4/DR5 antibodies was confirmed with a different format of ELISA. Wells of a microtitre plate were coated with DR5—Fc (SEQ ID NO:46). After blocking the wells with Blocking Buffer, culture supernatants of HEK293 cells were applied to the wells and incubated for 1 hr at room temperature. After washing wells with Wash Buffer, recombinant human DR4 extracellular region fused at the C-terminus to the human λ2 constant region (DR4-Cλ; SEQ ID NO:52) in ELISA Buffer was applied to the wells. After incubating the ELISA plate for 30 min at room temperature and washing the wells with Wash Buffer, bound DR4-Cλ was detected by HRP-conjugated goat anti-human X chain polyclonal antibody. Strong signals were observed for the 007/729 and 729/007 antibodies, indicating the presence of bispecific trimeric anti-DR4/DR5 IgG antibodies. No ELISA signals were observed with 007/007 or 729/729 antibodies.

### Example 15

### Generation, Expression and Characterization of a Multimeric IgG Antibody Against a Member of the TNF Receptor Superfamily

The TNF receptor superfamily is used in accordance with convention of authorities in the field, such as the Human Genome Organization (HUGO) and includes among others TNFRI (CD120a), TNFRII (CD120b), LtβR (lymphotoxin beta receptor), OX40 (CD134), CD40, FAS (CD95), CD27, CD30, 4-1BB (CD137), DR3, DR4 (CD261), DR5 (CD262), DR6 (CD358), DcR1 (CD263), DcR2 (CD264), DcR3, RANK (CD265), OPG, Fn14 (CD266), TACI (CD267), BAFFR (CD268), BCMA (CD269), HVEM (CD270), LNGFR (CD271), GITR (CD357), TROY, RELT, EDAR and XEDAR. Human forms of these receptors are preferred although homologs from other mammals or other species can also be used. Members of the superfamily are characterized by an extracellular domain of 2-6 cysteine rich motifs. Trimerization of membrane-bound TNF receptor superfamily members by their corresponding trimeric ligands triggers intracellular signal transduction (for review, see Hehlgans and Pfeffer, Immunology 115:1-20, 2005; Bossen et al., J. Biol. Chem. 281: 13964-13971, 2006; Tansey and Szymkowski, Drug Discovery Today 14: 23-24, 2009).

A monoclonal antibody against a member of the TNF receptor superfamily is generated using standard hybridoma technologies. The coding region of each of the VH and VL genes of the isolated monoclonal antibody is converted to an exon including a signal peptide-coding sequence, a splice donor signal, and flanking restriction enzyme sites as described above. Such constructed VH and VL genes are introduced into the corresponding sites of pHuYON007-THB, pHu11D1-THB or its derivative for expression of multimeric IgG of this invention as described above. The resulting multimeric IgG antibody is produced in mammalian cells, purified by protein A chromatography, analyzed for its size using a Superose 6 column as described above, and tested for its activity to modulate cellular responses using appropriate in vitro assays and animal efficacy models.

1. An antibody or fusion protein comprising first and second heavy chain constant regions associated with one another as a heterodimer, each chain comprising IgG CH2 and CH3 regions, and one of the chains comprising a homomultimerizing peptide linked to the C-terminus of the CH3 region.

2. The antibody or fusion protein of claim 1, wherein the homomultimerizing peptide is a trimerizing peptide.

3. The antibody or fusion protein of claim 1, wherein the homomultimerizing peptide is a dimerizing peptide.

4. The antibody or fusion protein of claim 1, wherein the homomultimerizing peptide is a tetramerizing peptide.

5. The antibody or fusion protein of claim 1, wherein the homomultimerizing peptide is a pentamerizing peptide.

6. The antibody or fusion protein of any preceding claim, which is an antibody further comprising first and second heavy chain variable regions fused to the first and second heavy chain constant regions and first and second light chains associated with the first and second heavy chains.

7. The antibody or fusion protein of claim 1, which is a dimeric fusion protein further comprising first and second heterologous proteins fused to the first and second heavy chain constant regions.

8. The antibody or fusion protein of claim 7, wherein the heterologous proteins are an extracellular domain of a receptor and/or a ligand to a receptor.

9. The fusion protein of claim 7, wherein the first and second constant regions further comprise and IgG hinge region and the heterologous proteins are linked to the IgG hinge regions of the first and second constant regions of the constant region via one or more flexible linkers, such as Gly-Gly-Ala-Ala.

10. The antibody or fusion protein of claim 1, wherein the first and second heavy chains incorporate modifications of natural IgG sequences promoting formation of the heterodimer.

11. The antibody or fusion protein of claim 10, wherein the first heavy chain incorporates a hole and the second heavy chain a knob, wherein coupling of the knob to the hole promotes formation of the heterodimer.

12. The antibody or fusion protein of claim 11, wherein the first and second heavy chains each comprises human IgG1 CH2 and CH3 regions and the first heavy chain has T366S, L368A and Y407V mutations, and the second heavy chain has a T366W mutation, amino acids being numbered by the EU numbering convention.

13. The antibody or fusion protein of claim 12, wherein the homomultimerizing peptide is a trimerizing peptide, which is linked to the CH3 domain of the second heavy chain.

14. The antibody or fusion protein of claim 14, wherein the trimerizing peptide comprises an isoleucine zipper or extracellular domain of a TNF superfamily member or tetranectin.

15. The antibody or fusion protein of claim 6, wherein the first and second heavy chain variable regions are the same.

16. The antibody or fusion protein of claim 6, wherein the first and second heavy chain variable regions are different.

17. The antibody or fusion protein of claim 16, wherein the first and second heavy chain variable regions are from antibodies binding to different targets.

18. The antibody or fusion protein of claim 6, wherein the first and second light chains are the same.

19. The antibody or fusion protein of claim 6, wherein the first and second light chains have different light chain variable regions.

20. The antibody or fusion protein of claim 19, wherein the first and second light chains have different light chain variable regions from antibodies binding to different targets.

21. The antibody or fusion protein of claim 1, wherein the homomultimerizing peptide is a trimerizing peptide and three units of the antibody or fusion protein form a trimer via association of the trimerizing peptides of the units.

22. The antibody or fusion protein of any one of claim 1, wherein the homomultimerizing peptide is a dimerizing peptide and two units of the antibody or fusion protein form a dimer via association of the dimerizing peptides of the units.

23. The antibody or fusion protein of any one of claim 1, wherein the homomultimerizing peptide is a tetramerizing peptide and four units of the antibody or fusion protein form a tetramer or fusion protein form a tetramer via association of the tetramerizing peptides of the units.

24. The antibody or fusion protein of any one of claim 1, wherein the homomultimerizing peptide is a pentamerizing peptide and five units of the antibody or fusion protein form a pentamer via association of the pentamerizing peptides of the units.

25. The antibody or fusion protein of any one of claim 1 in hexameric form, in which six units of the antibody or fusion protein form a hexamer via association of hexamerizing peptides of the units.

26-27. (canceled)

28. A multimeric complex including multiple units of an antibody or fusion protein, each unit comprising first and second heavy chain constants regions associated with one another as a heterodimer, each chain comprising IgG CH2 and CH3 regions, and one of the chains comprising a multimerizing peptide linked to the C-terminus of the CH3 region, wherein the units are associated as a the multimeric complex via multimerizing of the multimerizing peptides of the units.

29-30. (canceled)

31. The antibody or fusion protein of claim 1, wherein the human IgG CH1, and hinge (if present), CH2 and CH3 regions are human IgG1.

32. The antibody or fusion protein of claim 1, wherein the human IgG CH1, and hinge (if present), CH2 and CH3 regions are human IgG2 or human IgG4.

33-34. (canceled)

35. The antibody or fusion protein claim 1 that specifically binds to a Death Receptor family protein and induces apoptosis of cells bearing the protein.

36. The antibody or fusion protein or trimeric or multimeric complex of claim 35, wherein the Death Receptor family protein is DR4 or DR5.

37. The antibody or fusion protein claim 1 that specifically binds to a TNF receptor superfamily protein and induces apoptosis or cytostasis of cells bearing the protein.

38. The antibody or fusion protein of claim 1 that specifically binds to and agonizes OX40, CD40, FAS, CD27, CD30, 4-1BB, DR3, DR4, DR5, DR6, DcR1, DcR2, DcR3, RANK, OPG, Fn14, TACI, BAFFR, BCMA, HVEM, LNGFR, GITR, TROY, RELT, EDAR or XEDAR thereby stimulating an immune response.

39. The antibody or fusion protein of claim 1, which specifically binds protein G, specifically binds protein A, exhibits ADCC, CDC and/or opsonization.

40-42. (canceled)

43. The antibody or fusion protein of claim 1, which is a humanized, chimeric, veneered or human antibody.

44. The antibody or fusion protein of claim 1 that specifically binds the extracellular domain of a receptor.

45. The antibody or fusion protein ofclaim 44, which is an antibody that specifically binds to CD79a, CD30, DR5 or DR4.

46. The antibody or fusion protein of claim 1, which is a fusion protein comprising an extracellular domain of a TNF-alpha receptor, LFA-3 or an IL-1 receptor.

47. The antibody or fusion protein of claim 1, which is a fusion protein or trimeric complex thereof comprising a TRAIL protein.

48. The antibody or fusion protein claim 1 that is conjugated to a cytotoxic moiety.

49. (canceled)

50. The antibody or fusion of claim 1, which is an antibody or fusion protein that specifically binds to CD40, OX40, 4-1BB, GITR or CD27.

51-56. (canceled)


SEQUENCE LISTING (68 sequences)

SEQ ID NO: 1 - protein, 452 aa, Artificial Sequence
  Synthesized
ASGTEAAAATPSKVWGSSAGRIEPRGGGRGALPTSMGQHGPSARARAGRAPGPRPAREASPRLRVHKTFKFVVVGVLLQVVPSSAATIKLHDQSIGTQQWEHSPLGELCPPGSHRSEHPGACNRCTEGVGYTNASNNLFACLPCTACKSDEEERSPCTTTRNTACQCKPGTFRNDNSAEMCRKCSTGCPRGMVKVKDCTPWSDIECVHKESGNGHNTGGGEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 138 aa, Artificial Sequence
  Synthesized
MNRLTSSLLLLIVPAYVLSQVTLRESGPALVKPTQTLTLTCTFSGFSLSTSGMGVSWIRQPPGKALEWLAHIYWDDDKRYNPSLKSRLTISKDTSKNQVVLTMTNMDPVDTATYYCTRRGEYGNFDYWGQGTLVTVSS

SEQ ID NO: 3 - protein, 128 aa, Artificial Sequence
  Synthesized
MAWISLILSLLALSSGAISQTVVTQEPSFSVSPGGTVTLTCRSSSGAVTTSNFANWVQQTPGQAPRGLIGGTNNRAPGVPDRFSGSLLGNKAALTITGAQADDESDYYCALWYSNHWVFGGGTKLTVL

SEQ ID NO: 4 - protein, 330 aa, Artificial Sequence [sequence omitted]

SEQ ID NO: 5 - protein, 330 aa, Artificial Sequence
  Synthesized
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 6 - protein, 330 aa, Artificial Sequence
  Synthesized
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 373 aa, Artificial Sequence
  Synthesized
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKSGGGSGGGSMKQIEDKIEEILSKIYHIENEIARIKKLIGERAG

SEQ ID NO: 8 - protein, 493 aa, Artificial Sequence
  Synthesized
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKSGGGGSVRSSSRTPSDKPVAHVVANPQAEGQLQWLNRRANALLANGVELRDNQLVVPSEGLYLIYSQVLFKGQGCPSTHVLLTHTISRIAVSSQTKVNLLSAIKSPCQRETPEGAEAKPWYEPIYLGGVFQLEKGDRLSAEINRPDYLDFAESGQVYFGIIAL

SEQ ID NO: 9 - protein, 281 aa, Artificial Sequence
  Synthesized
MAWISLILSLLALSSGAISQTVVTQEPSFSVSPGGTVTLTCRSSSGAVTTSNFANWVQQTPGQAPRGLIGGTNNRAPGVPDRFSGSLLGNKAALTITGAQADDESDYYCALWYSNHWVFGGGTKLTVLGGGGSGGGGSGGGGSQVTLRESGPALVKPTQTLTLTCTFSGFSLSTSGMGVSWIRQPPGKALEWLAHIYWDDDKRYNPSLKSRLTISKDTSKNQVVLTMTNMDPVDTATYYCTRRGEYGNFDYWGQGTLVTVSSTGGGEPKSCDKTHTCPPCP

SEQ ID NO: 10 - protein, 592 aa, Artificial Sequence
  Synthesized
QTVVTQEPSFSVSPGGTVTLTCRSSSGAVTTSNFANWVQQTPGQAPRGLIGGTNNRAPGVPDRFSGSLLGNKAALTITGAQADDESDYYCALWYSNHWVFGGGTKLTVLGGGGSGGGGSGGGGSQVTLRESGPALVKPTQTLTLTCTFSGFSLSTSGMGVSWIRQPPGKALEWLAHIYWDDDKRYNPSLKSRLTISKDTSKNQVVLTMTNMDPVDTATYYCTRRGEYGNFDYWGQGTLVTVSSTGGGEPKSCDKTHTCPPCPASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 11 - protein, 635 aa, Artificial Sequence
  Synthesized
QTVVTQEPSFSVSPGGTVTLTCRSSSGAVTTSNFANWVQQTPGQAPRGLIGGTNNRAPGVPDRFSGSLLGNKAALTITGAQADDESDYYCALWYSNHWVFGGGTKLTVLGGGGSGGGGSGGGGSQVTLRESGPALVKPTQTLTLTCTFSGFSLSTSGMGVSWIRQPPGKALEWLAHIYWDDDKRYNPSLKSRLTISKDTSKNQVVLTMTNMDPVDTATYYCTRRGEYGNFDYWGQGTLVTVSSTGGGEPKSCDKTHTCPPCPASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKSGGGSGGGSMKQIEDKIEEILSKIYHIENEIARIKKLIGERAG

SEQ ID NO: 12 - protein, 34 aa, Artificial Sequence
  Synthesized
MKQIEDKIEEILSKIYHIENEIARIKKLIGERAG

SEQ ID NO: 13 - protein, 157 aa, Homo sapiens
VRSSSRTPSDKPVAHVVANPQAEGQLQWLNRRANALLANGVELRDNQLVVPSEGLYLIYSQVLFKGQGCPSTHVLLTHTISRIAVSSQTKVNLLSAIKSPCQRETPEGAEAKPWYEPIYLGGVFQLEKGDRLSAEINRPDYLDFAESGQVYFGIIAL

SEQ ID NO: 14 - protein, 186 aa, Artificial Sequence [sequence omitted]

SEQ ID NO: 15 - protein, 137 aa, Artificial Sequence
  Synthesized
MGRLTSSFLLLIVPAYVLSQVTLKESGPGILQPSQTLSLTCSFSGFSLSTSGVGVGWIRQPSGKGLEWLAHIWWDDDKYYNTALKSGLTISKDTSKNQVFLKIASVDTADTATYYCARIDWDGIAYWGQGTLVTVSA

SEQ ID NO: 16 - protein, 7 aa, Artificial Sequence
  Synthesized
TSGVGVG

SEQ ID NO: 17 - protein, 16 aa, Artificial Sequence
  Synthesized
HIWWDDDKYYNTALKS

SEQ ID NO: 18 - protein, 8 aa, Artificial Sequence
  Synthesized
IDWDGIAY

SEQ ID NO: 19 - protein, 128 aa, Artificial Sequence
  Synthesized
MDFQVQIFSFLLISASVIMSRGQIVLSQSPAILSTSPGEKVTMTCRASSSVSYMHWYQEKPGSSPKPWIYATSNLASGVPARFSGSGSGTSYSLTISRVEAEDAATYYCQQWSSNPWTFGGGTKLEIK

SEQ ID NO: 20 - protein, 10 aa, Artificial Sequence
  Synthesized
RASSSVSYMH

SEQ ID NO: 21 - protein, 7 aa, Artificial Sequence
  Synthesized
ATSNLAS

SEQ ID NO: 22 - protein, 9 aa, Artificial Sequence
  Synthesized
QQWSSNPWT

SEQ ID NO: 23 - protein, 137 aa, Artificial Sequence
  Synthesized
MGRLTSSFLLLIVPAYVLSQVTLRESGPALVKPTQTLTLTCTFSGFSLSTSGVGVGWIRQPPGKALEWLAHIWWDDDKYYNTALKSGLTISKDTSKNQVVLTMTNMDPVDTATYYCARIDWDGIAYWGQGTLVTVSS

SEQ ID NO: 24 - protein, 128 aa, Artificial Sequence
  Synthesized
MDFQVQIFSFLLISASVIMSRGEIVLTQSPATLSLSPGERATLSCRASSSVSYMHWYQQKPGQAPRPWIYATSNLASGIPARFSGSGSGTDYTLTISSLEPEDFAVYYCQQWSSNPWTFGGGTKVEIK

SEQ ID NO: 25 - DNA, 448 bp, Artificial Sequence
  Synthesized
actagtaccaccatgggcagacttacttcttcattcttgctgctgattgtccctgcatatgtcctgtcccaggttactctgagagagtctggccctgccctggtgaagcccacccagaccctcactctgacttgtactttctctgggttttcactgagcacttctggtgtgggagtcggctggattcgtcagcctccagggaaggctctcgagtggctggcacacatttggtgggatgatgataagtactataacacagccctgaagagcgggctcacaatctccaaggatacctccaaaaaccaggtcgtcctcaccatgaccaatatggaccctgtggatactgccacatactactgtgctcgaattgactgggatgggattgcttactggggccaagggactctggtcactgtctcttcaggtgagtctgctgtactggaagctt

SEQ ID NO: 26 - DNA, 421 bp, Artificial Sequence
  Synthesized
gctagcaccaccatggattttcaagtgcagattttcagcttcctgctgatcagtgcttcagtcatcatgtccagaggagaaattgttctcacccagtctccagcaaccctgtctctgtctccaggggagagggccacactgtcttgcagggccagctcaagtgttagttacatgcactggtaccagcagaagccaggacaggcccccagaccctggatttatgccacatccaacctggcttctggaatccctgctcgcttcagtggcagtgggtctgggaccgattacactctcacaatcagcagcctggagcctgaagattttgccgtttattactgccagcagtggagtagtaacccctggaccttcggtggaggcaccaaggtggaaatcaaacgtaagtgcactttcctaagaattc

SEQ ID NO: 27 - protein, 409 aa, Artificial Sequence
  Synthesized
EPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRTGGGEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 137 aa, Artificial Sequence
  Synthesized
MDIRLSLAFLVLFIKGVQCEVQLVESGGGLVQPGRSMKLSCAASGFTFSYFPMAWVRQAPTKGLEWVATISTSGGNIYYRDSVKGRFTISRDNAKSTLYLQMNSLRSEDTATYYCTRDTAPYYFDYWGQGVMVTVSS

SEQ ID NO: 29 - protein, 5 aa, Artificial Sequence
  Synthesized
YFPMA

SEQ ID NO: 30 - protein, 17 aa, Artificial Sequence
  Synthesized
TISTSGGNIYYRDSVKG

SEQ ID NO: 31 - protein, 9 aa, Artificial Sequence
  Synthesized
DTAPYYFDY

SEQ ID NO: 32 - protein, 127 aa, Artificial Sequence
  Synthesized
MRAHAQFLGLLLLWFPGARCDIQMTQSPSSISVSLGDRFTITCRASQDIGNYLNWYQQKPEKSPKLMIYRATNLEDGVPSRFSGSRSGSDYSLTINSLESEDTGFYFCVQHKQYPLTFGSGTKLEIK

SEQ ID NO: 33 - protein, 11 aa, Artificial Sequence
  Synthesized
RASQDIGNYLN

SEQ ID NO: 34 - protein, 7 aa, Artificial Sequence
  Synthesized
RATNLED

SEQ ID NO: 35 - protein, 9 aa, Artificial Sequence
  Synthesized
VQHKQYPLT

SEQ ID NO: 36 - protein, 136 aa, Artificial Sequence
  Synthesized
MDIRLSLAFLVLFIAGVQCEVQLVESGGGLVQPGGSLRLSCAASGFTFSYFPMAWVRQAPGKGLEWVATISTSGGNIYYRDSVKGRFTISRDNAKNSLYLQMNSLRAEDTAVYYCTRDTAPYYFDYWGQGTMVTVS

SEQ ID NO: 37 - protein, 127 aa, Artificial Sequence
  Synthesized
MRAHAQFLGLLLLWFPGARCDIQMTQSPSSLSASVGDRVTITCRASQDIGNYLNWYQQKPGKAPKLLIYRATNLEDGVPSRFSGSGSGTDYTLTISSLQPEDFATYYCVQHKQYPLTFGGGTKVEIK

SEQ ID NO: 38 - DNA, 447 bp, Artificial Sequence
  Synthesized
actagtaccaccatggatatcaggctcagcttggctttccttgtccttttcatcgcaggcgtccagtgtgaagtgcaactcgtcgagtctgggggcggactcgtgcagcctggaggctccctgagactctcctgtgcagcctcaggattcactttcagttactttccaatggcctgggtccgccaggctccaggcaagggtctggagtgggtcgcaaccattagtaccagtggaggcaatatctattatcgagactccgtgaagggccgattcactatctccagagataatgcaaaaaactccctgtacctgcaaatgaacagtctgagggctgaggacacagccgtttattactgtacaagagataccgctccctactactttgattactggggccaaggaaccatggtcacagtctcctcaggtaagatgggctttcctaagctt

SEQ ID NO: 39 - DNA, 417 bp, Artificial Sequence
  Synthesized
gctagcaccaccatgagggcccatgctcagtttcttgggctgttgttgctctggtttccaggagccagatgcgacatccagatgacccagtctccatcctccctgtctgcctctgtgggagacagagtcactattacttgtcgggcaagtcaagacattggaaactatttgaactggtaccagcagaaaccaggaaaagctcctaagctcctgatttatcgtgctaccaacttggaagatggggtcccatcaagattcagtggcagtgggtctgggacagattatactctcaccatcagcagcctgcagcctgaagatttcgcaacctactactgtgtccagcataaacagtatcccctcaccttcggaggcgggaccaaggtggagatcaaacgtaagtgcactttcctagaattc

SEQ ID NO: 40 - protein, 146 aa, Artificial Sequence
  Synthesized
GDQNPQIAAHVISEASSKTTSVLQWAETGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 41 - protein, 484 aa, Artificial Sequence
  Synthesized
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGSGGGSGGGSGDQNPQIAAHVISEASSKTTSVLQWAETGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 42 - protein, 34 aa, Artificial Sequence
  Synthesized
MKQLEDKVEELLSKNYHLENEVARLKKLVGERAG

SEQ ID NO: 43 - protein, 374 aa, Artificial Sequence
  Synthesized
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKSGGGSGGGSHMKQLEDKVEELLSKNYHLENEVARLKKLVGERAG

SEQ ID NO: 44 - protein, 34 aa, Artificial Sequence
  Synthesized
MKQIEDKLEEILSKLYHIENELARIKKLLGERAG

SEQ ID NO: 45 - protein, 374 aa, Artificial Sequence
  Synthesized
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKSGGGSGGGSHMKQIEDKLEEILSKLYHIENELARIKKLLGERAG

SEQ ID NO: 46 - protein, 393 aa, Artificial Sequence
  Synthesized
ALITQQDLAPQQRAAPQQKRSSPSEGLCPPGHHISEDGRDCISCKYGQDYSTHWNDLLFCLRCTRCDSGEVELSPCTTTRNTVCQCEEGTFREEDSPEMCRKCRTGCPRGMVKVGDCTPWSDIECVHKESGTKHSGEAPAVEETVTSSPGTPASPCSTGGGEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 47 - protein, 139 aa, Artificial Sequence
  Synthesized
MEWCWVFLFLLSVTAGVHSQVQLVQSGAEVKKPGASVKVSCKASGYTFTDYIIHWVRQAPGQGLEWIGWFYPGNNNIKSNEKFKDRVTLTADTSTSTVYMELSSLRSEDTAVYYCARNEDNYGNFFGYWGQGTLVTVSS

SEQ ID NO: 48 - protein, 127 aa, Artificial Sequence
  Synthesized
MESQIQAFVFVFLWLSGVDGDIQMTQSPSSLSASVGDRVTITCKASQDVNTAAAWYQQKPGKAPKLLIYWASTRHTGVPSRFSGSGSGTDYTLTISSLQPEDFATYYCQQHYSTPYTFGQGTKLEIK

SEQ ID NO: 49 - protein, 591 aa, Artificial Sequence
  Synthesized
DIQMTQSPSSLSASVGDRVTITCKASQDVNTAAAWYQQKPGKAPKLLIYWASTRHTGVPSRFSGSGSGTDYTLTISSLQPEDFATYYCQQHYSTPYTFGQGTKLEIKGGGGSGGGGSGGGGSQVQLVQSGAEVKKPGASVKVSCKASGYTFTDYIIHWVRQAPGQGLEWIGWFYPGNNNIKSNEKFKDRVTLTADTSTSTVYMELSSLRSEDTAVYYCARNEDNYGNFFGYWGQGTLVTVSSTGGGEPKSCDKTHTCPPCPASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLSSKLTVDKSRWQQGNVFSCSVMHEALHNHYTQKSLSLSPGK

SEQ ID NO: 50 - protein, 634 aa, Artificial Sequence
  Synthesized
DIQMTQSPSSLSASVGDRVTITCKASQDVNTAAAWYQQKPGKAPKLLIYWASTRHTGVPSRFSGSGSGTDYTLTISSLQPEDFATYYCQQHYSTPYTFGQGTKLEIKGGGGSGGGGSGGGGSQVQLVQSGAEVKKPGASVKVSCKASGYTFTDYIIHWVRQAPGQGLEWIGWFYPGNNNIKSNEKFKDRVTLTADTSTSTVYMELSSLRSEDTAVYYCARNEDNYGNFFGYWGQGTLVTVSSTGGGEPKSCDKTHTCPPCPASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKSGGGSGGGSMKQIEDKIEEILSKIYHIENEIARIKKLIGERAG

SEQ ID NO: 51 - protein, 267 aa, Artificial Sequence
  Synthesized
ALITQQDLAPQQRAAPQQKRSSPSEGLCPPGHHISEDGRDCISCKYGQDYSTHWNDLLFCLRCTRCDSGEVELSPCTTTRNTVCQCEEGTFREEDSPEMCRKCRTGCPRGMVKVGDCTPWSDIECVHKESGTKHSGEAPAVEETVTSSPGTPASPCSTGGGGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTESS

SEQ ID NO: 52 - protein, 326 aa, Artificial Sequence
  Synthesized
ASGTEAAAATPSKVWGSSAGRIEPRGGGRGALPTSMGQHGPSARARAGRAPGPRPAREASPRLRVHKTFKFVVVGVLLQVVPSSAATIKLHDQSIGTQQWEHSPLGELCPPGSHRSEHPGACNRCTEGVGYTNASNNLFACLPCTACKSDEEERSPCTTTRNTACQCKPGTFRNDNSAEMCRKCSTGCPRGMVKVKDCTPWSDIECVHKESGNGHNTGGGGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTESS

SEQ ID NO: 53 - protein, 98 aa, Artificial Sequence
  Synthesized
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 54 - protein, 15 aa, Artificial Sequence
  Synthesized
EPKSCDKTHTCPPCP

SEQ ID NO: 55 - protein, 110 aa, Artificial Sequence
  Synthesized
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 56 - protein, 107 aa, Artificial Sequence
  Synthesized
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 57 - protein, 98 aa, Artificial Sequence
  Synthesized
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTV

SEQ ID NO: 58 - protein, 12 aa, Artificial Sequence
  Synthesized
ERKCCVECPPCP

SEQ ID NO: 59 - protein, 109 aa, Artificial Sequence
  Synthesized
APPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTK

SEQ ID NO: 60 - protein, 107 aa, Artificial Sequence
  Synthesized
GQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 61 - protein, 98 aa, Artificial Sequence
  Synthesized
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRV

SEQ ID NO: 62 - protein, 62 aa, Artificial Sequence
  Synthesized
ELKTPLGDTTHTCPRCPEPKSCDTPPPCPRCPEPKSCDTPPPCPRCPEPKSCDTPPPCPRCP

SEQ ID NO: 63 - protein, 110 aa, Artificial Sequence
  Synthesized
APELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTK

SEQ ID NO: 64 - protein, 107 aa, Artificial Sequence
  Synthesized
GQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 65 - protein, 98 aa, Artificial Sequence
  Synthesized
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRV

SEQ ID NO: 66 - protein, 12 aa, Artificial Sequence
  Synthesized
ESKYGPPCPSCP

SEQ ID NO: 67 - protein, 110 aa, Artificial Sequence
  Synthesized
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAK

SEQ ID NO: 68 - protein, 107 aa, Artificial Sequence
  Synthesized
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK